SEQUENCE LISTING (3 sequences)

SEQ ID NO: 1 - protein, 303 aa, Homo sapiens
  REGION 1..303: note = Description of sequence: Human DNASE1L3 original amino acid sequence
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSK

SEQ ID NO: 2 - DNA, 911 bp, Homo sapiens
  misc_feature 1..911: note = Description of sequence: Human DNASE1L3 original nucleic acid sequence
  source 1..911: mol_type = other DNA
atgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagagcaaacg

SEQ ID NO: 3 - DNA, 915 bp, synthetic construct
  misc_feature 1..915: note = Description of sequence: Codon optimized human DNASE1L3 nucleic acid sequence:
  source 1..915: mol_type = other DNA
atgtctagggaacttgctccactcctcttgctcctcttgtctatccactctgctcttgccatgcgtatctgctctttcaacgtcaggtctttcggtgaaagcaagcaggaagacaagaacgctatggacgtcatcgtgaaggtcatcaagaggtgcgacatcatcctcgtgatggaaatcaaggactcaaacaacagaatctgtcctatcctgatggagaagctcaaccgtaactccagacgtggaatcacctacaactacgtcatctctagcagattgggtcgtaacacttacaaggaacaatacgccttcttgtacaaggagaagctggtgtcagtcaagaggtcctaccactaccacgactaccaggacggcgacgctgacgtgttctctcgcgaaccattcgtcgtgtggttccagagccctcacaccgccgtgaaggacttcgtcatcatccctttgcacaccactcccgaaacctcagtgaaggagatcgacgaactggtggaggtctacactgacgtcaagcacagatggaaggctgagaacttcatcttcatgggtgacttcaacgctggctgctcttacgtgcccaagaaggcctggaagaacatcaggctgcgcaccgacccacgtttcgtgtggctcatcggcgaccaggaagacaccactgtcaagaagagcactaactgtgcctacgacaggatcgtgctgcgcggacaggagatcgtctcatccgtcgtgcctaagtcaaactccgtcttcgacttccaaaaggcttacaagctcactgaagaggaagccttggacgtgagcgaccacttccccgtcgaattcaagctccaatctagcagagctttcacaaacagtaaaaaatcagtaaccctgcgtaagaagactaagagtaaacgtagc

(19) United States
(12) Patent Application Publication
(10) Pub. No.: US 2026/0176705 A1
(43) Pub. Date: Jun. 25, 2026

(54) METHODS AND COMPOSITIONS FOR ENRICHING CIRCULATING CELL-FREE NUCLEIC ACIDS IN A BIOLOGICAL SAMPLE

(71) Applicants: Centre for Novostics, Shatin (HK); Tencent Healthcare (Shenzhen) Co., Ltd., Nanshan District (CN)

(72) Inventors: Yuk-Ming Dennis Lo, Homantin (CN); Kwan Chee Chan, Jordan (CN); Peiyong Jiang, Pak Shek Kok (CN); Pak Hang Peter Cheung, Kwun Tong (CN); Man Tat Alexander Ng, Mid-Ievels (HK); Wei Liu, Shenzhen (CN); Hou Tim Lai, Zhuhai (CN); Biaobin Jiang, Shenzhen (CN)

(21) Appl. No.: 19/397,506

(22) Filed: Nov. 21, 2025

Related U.S. Application Data
(60) Provisional application No. 63/747,794, filed on Jan. 21, 2025.

(30) Foreign Application Priority Data
Nov. 25, 2024 (WO) PCT/CN2024/134338
Nov. 19, 2025 (WO) PCT/CN2025/136013

(51) Int. Cl. C12Q 1/6886; C12Q 1/6806; C12Q 1/6883

(52) U.S. Cl. CPC C12Q 1/6886; C12Q 1/6806; C12Q 1/6883; C12Q 2600/156

(57) ABSTRACT

The present disclosure provides methods and compositions for improving the quantity and quality of cfDNAs in a bodily fluid of a subject by inhibiting the nuclease activity in the subject. The methods and compositions disclosed herein are particularly useful for subjects who have been identified that liquid biopsies are needed.

### CROSS-REFERENCE TO RELATED APPLICATIONS

The present application claims priority to International Application No. PCT/CN2025/136013, filed Nov. 19, 2025, U.S. Provisional Application No. 63/747,794, filed Jan. 21, 2025, and International Application No. PCT/CN2024/134338, filed Nov. 25, 2024, the disclosures of which are herein incorporated by reference in their entirety for all purposes.

### SEQUENCE LISTING SUBMITTED VIA ELECTRONIC FILING SYSTEM

The instant application contains a Sequence Listing which has been filed electronically in XML format and is hereby incorporated by reference in its entirety. Said XML copy, created on Nov. 21, 2025, is named 117655-8020US1-1529253_ST26.xml and is 6,927 bytes in size.

### BACKGROUND

Circulating cell-free DNA (cfDNA) has emerged as a promising noninvasive biomarker for various clinical applications, including cancer diagnostics, prenatal testing, disease/condition monitoring, and therapeutic management.

However, its limited quantity and quality in bodily fluids, such as blood, often lead to reduced sensitivity in detecting abnormalities, particularly in early-stage conditions. This scarcity poses significant challenges for accurate diagnosis and monitoring. Therefore, the development of new methods and compositions to enrich circulating cfDNA in biological samples is essential.

### BRIEF SUMMARY

The present disclosure provides methods, compositions, and systems for increasing circulating cell-free DNA (cfDNA) level in a biological sample, such as a bodily fluid, of a subject. Certain compositions can be administered to a subject to increase an amount of cfDNA in a resulting sample. Such a sample can be assayed and measured values used to detect various properties of the sample, such as a sequence imbalance such as a copy number aberration of a chromosomal region or an entire chromosome (e.g., an aneuploidy), a pathology such as cancer, or a fractional concentration of clinically-relevant DNA. Various markers of cfDNA can be measured in such a sample, e.g., as described herein.

In one aspect, the present disclosure provides a method of increasing cell-free DNA (cfDNA) level in a bodily fluid of a subject. In some embodiments, the method comprises administering to the subject a therapeutically effective amount of a nuclease inhibitor, wherein the subject has been identified for a liquid biopsy to be assayed for cfDNA, and wherein the nuclease inhibitor binds to a nuclease and inhibits cleavage of the cfDNA in the bodily fluid, thereby increasing the cfDNA level in the subject. In some embodiments, the nuclease inhibitor is an antibiotic.

In some embodiments, the antibiotic is a cephalosporin, sulfonamide, tetracycline, rifamycin, or polyene macrolide. In some embodiments, the cephalosporin is ceforanide, cefuroxime sodium, cefamandole sodium, cefaclor, cefdinir, or a derivative thereof. In some embodiments, the sulfonamide is sulfadiazine, sulphapyridine, or a derivative thereof. In some embodiments, the tetracycline is minocycline, doxycycline, demeclocycline, tetracycline, omadacycline, sarecycline, or a derivative thereof. In some embodiments, the rifamycin is rifaximin, rifabutin, rifamycin, rifampin (rifampicin), rifapentine, rifalazil, or a derivative thereof.

In some embodiments, the polyene macrolide is amphotericin B, nystatin, natamycin, or a derivative thereof.

In some embodiments, the nuclease is a deoxyribonuclease (DNase). In some embodiments, the DNase is deoxyribonuclease 1 (DNase1) or deoxyribonuclease 1-like 1 (DNase1L1), DNase1L2, DNase1L3, or DNA fragmentation factor subunit beta (DFFB). In some embodiments, the DNase is DNase1L3. In some embodiments, the nuclease inhibitor binds to a catalytic pocket of the nuclease.

In some embodiments, the nuclease inhibitor is administered orally, intravenously, intradermally, intramuscularly, intraperitoneally, subcutaneously, intranasally, epidurally, sublingually, intracerebrally, intravaginally, trans-dermally, rectally, by inhalation, or topical administration. In some embodiments, the therapeutically effective amount of the nuclease inhibitor is about 1-50 uM per kilogram body weight, optionally about 10-30 uM per kilogram body weight, and optionally about 20 uM per kilogram body weight. In some embodiments, the cfDNA level is increased by up to 2, 3, 4, 5, 10, 25, 50, 100, 200, 300, 400, 500, 600, 700, 800, 900, or 1000-fold, as compared to a cfDNA level of a subject without administering the nuclease inhibitor.

In some embodiments, the bodily fluid is selected from the group consisting of blood, plasma, serum, urine, vaginal fluid, fluid from a hydrocele (e.g., of the testis), vaginal flushing fluids, pleural fluid, ascitic fluid, cerebrospinal fluid (CSF), saliva, sweat, tears, sputum, bronchoalveolar lavage fluid, peritoneal fluid, discharge fluid from the nipple, aspiration fluid from different parts of the body (e.g., thyroid, breast), intraocular fluids (e.g., the aqueous humor), amniotic fluid, aqueous humor, ascites, bone marrow fluid, lymphatic fluid, synovial fluid, interstitial fluid, prostate fluid, semen, mucus, gastric acid, bile, pus, cerumen, breast milk, cowper's fluid or pre-ejaculatory fluid, female ejaculate, hair oil, cyst fluid, dialysis fluid, pericardial fluid, chyme, chyle, menses, sebum, vomit, mucosal secretion, stool water, pancreatic juice, lavage fluids from sinus cavities, bronchopulmonary aspirates, blastocoel fluid, urinary tract secretions, urethral secretions, bladder secretions, prostate secretions, vesical secretions, meconium, and umbilical cord fluid. In some embodiments, the bodily fluid comprises blood, serum, and/or plasma.

In some embodiments, the subject is a human. In some embodiments, the subject is pregnant, and wherein the cfDNA comprises a mixture of fetal and subject's DNA. In some embodiments, the subject has, is suspected of having, or is at risk for a disease detectable using the cfDNA. In some embodiments, the disease is cancer, a premalignant condition, an inflammatory disease, an immune disease, an autoimmune disease or disorder, a cardiovascular disease or disorder, neurological disease or disorder, infectious disease, or pain. In some embodiments, the disease is cancer, and wherein the cancer is selected from the group consisting of colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, gallbladder cancer, brain cancer, peritoneal cancer, lymphoma, leukemia, multiple myeloma, neuroblastoma, osteosarcoma, head and neck cancer, oral cancer, nasopharyngeal cancer, skin cancer and soft tissue sarcoma.

In another aspect, the present disclosure provides a method of assaying cfDNA from a bodily fluid of a subject. In some embodiments, the method comprises (a) increasing cfDNA level in the bodily fluid of the subject by conducting the method described herein; and (b) assaying the cfDNA.

In some embodiments, the method further comprises collecting the cfDNA from the bodily fluid prior to the assaying step. In some embodiments, the cfDNA is collected within 1 minute to 24 hours after the administering step. In some embodiments, the assaying step comprises sequencing, amplification, hybridization, gel electrophoresis, chromatography, immunoassay, enzyme immunoassay (EIA), enzyme-linked immunosorbent assay (ELISA), enzyme-linked oligonucleotide assay (ELONA), affinity isolation, immunoprecipitation, Western blot, flow cytometry, and any combination thereof. In some embodiments, the assaying step is conducted at least 1 minute, 2 minutes, 3 minutes, 4 minutes, 5 minutes, 10 minutes, 15 minutes, 30 minutes, 1 hour, 2 hours, 3 hours, 4 hours, 5 hours, 10 hours, 15 hours, 20 hours, or 24 hours after the administering step.

In some embodiments, the method further comprises (c) identifying the subject as having a disease based on a property of the cfDNA indicating the disease, wherein the property of the cfDNA is determined using assay results detected in the assaying step. In some embodiments, the method further comprises (d) administering to the subject one or more treatments for the disease.

In another aspect, the present disclosure provides a method of inhibiting activity of a nuclease in a subject. In some embodiments, the method comprises administering to the subject a therapeutically effective amount of a nuclease inhibitor that binds to the nuclease, thereby inhibiting the nuclease activity in the subject, wherein the subject has been identified for a liquid biopsy to be assayed for cfDNA. In some embodiments, inhibiting the nuclease activity results in optimizing the level and/or size of cfDNA to be assayed in the liquid biopsy.

In some embodiments, the nuclease inhibitor is an antibiotic. In some embodiments, the antibiotic is selected from the group consisting of ceforanide, cefuroxime sodium, cefamandole sodium, cefaclor, cefdinir, sulfadiazine, sulphapyridine, minocycline, doxycycline, demeclocycline, tetracycline, omadacycline, sarecycline, rifaximin, rifabutin, rifamycin, rifampin (rifampicin), rifapentine, rifalazil, amphotericin B, nystatin, natamycin, and a derivative thereof. In some embodiments, the antibiotic comprises ceforanide, cefuroxime sodium, minocycline, and/or rifamycin. In some embodiments, the nuclease inhibitor is administered orally, intravenously, intradermally, intramuscularly, intraperitoneally, subcutaneously, intranasally, epidurally, sublingually, intracerebrally, intravaginally, trans-dermally, rectally, by inhalation, or topical administration.

In some embodiments, the nuclease is a deoxyribonuclease (DNase). In some embodiments, the DNase is deoxyribonuclease 1 (DNase1), deoxyribonuclease 1-like 1 (DNase1L1), DNase1L2, DNase1L3, or DNA fragmentation factor subunit beta (DFFB) nuclease. In some embodiments, the nuclease activity is inhibited by about 5%, 10%, 15%, 20%, 25%, 30%, 35%, 40%, 45%, 50%, 55%, 60%, 65%, 70%, 75%, 80%, 85%, 90%, 95%, 99%, or 100%, as compared to the nuclease activity in the absence of the nuclease inhibitor.

In another aspect, the present disclosure also provides a kit comprising a reagent for carrying out the methods described herein. In some embodiments, the reagent of the kit comprises a nuclease inhibitor and a pharmaceutically acceptable adjuvant. In some embodiments, the pharmaceutically acceptable adjuvant comprises divalent cations Ca2+ and/or Mg²⁺.

In another aspect, the present disclosure provides a method of producing a recombinant human deoxyribonuclease 1-like 3 (DNASE1L3). In some embodiments, the method of producing a recombinant human DNASE1L3 comprises (a) introducing a polynucleotide encoding human DNASE1L3 into an insect cell via a baculovirus expression vector; and (b) expressing the recombinant human DNASE1L3 in an insect cell culture. In some embodiments, the polynucleotide comprises a nucleic acid sequence having at least 80% identity to SEQ ID NO: 2 or SEQ ID NO: 3.

In some embodiments, the method further comprises (c) purifying the recombinant human DNASE1L3 from the insect cell culture. In some embodiments, the purification comprises affinity chromatography, ion-exchange chromatography, size-exclusion chromatography, or any combination thereof.

In another aspect, the present disclosure provides a method of assaying deoxyribonuclease (DNase) activity in vitro. In some embodiments, the method of assaying DNase activity comprises performing a set of assays having different probe concentrations or different DNase concentrations. In some embodiments, performing each of the set of assays includes: (a) contacting a fluorescent probe with a DNase in a reaction mixture, the fluorescent probe or the DNase at a respective concentration of a series of concentrations; (b) incubating the reaction mixture at a specified temperature from 35° C. to 40° C.; and (c) measuring fluorescence intensity, thereby obtaining a linear fluorescence response over the series of concentrations. In some embodiments, the specified temperature is about 37° C.

In some embodiments, when performing the method of assaying DNase activity, the concentration of the fluorescent probe in the reaction mixture is about 10-100 nM. In some embodiments, the concentration of the fluorescent probe in the reaction mixture is about 50 nM.

In some embodiments, the fluorescent probe comprises a DNA oligonucleotide linked with a fluorophore at the 5 terminus and a quencher at the 3 terminus. In some embodiments, the DNA oligonucleotide is a single stranded DNA oligonucleotide. In some embodiments, the DNA oligonucleotide is a double stranded DNA oligonucleotide. In some embodiments, the fluorophore is FAM. In some embodiments, the quencher is IABkFQ. In some embodiments, the fluorescent probe does not comprise an internal quencher.

In some embodiments, when performing the method of assaying DNase activity, the concentration of the DNase in the reaction mixture is about 10-2000 pM. In some embodiments, the DNase is a recombinant human DNASE1L3.

In some embodiments, the reaction mixture comprises Tris-HCl, MgCl₂, and/or CaCl₂). In some embodiments, the concentration of Tris-HCl in the reaction mixture is about 20 mM, the concentration of MgCl₂ in the reaction mixture is about 2 mM, and/or the concentration of CaCl₂) in the reaction mixture is about 2 mM. In some embodiments, the pH value of the reaction mixture is about between 6 and 9, preferably about 7.5.

In some embodiments, step (c) of the method of assaying DNase activity comprises measuring fluorescence intensity by exciting the fluorophore (e.g., FAM or equivalent) at a wavelength in the range of 450-500 nm (preferably 460-485 nm, most preferably 465-475 nm or approximately 468 nm), and detecting the emitted fluorescence at a wavelength in the range of 510-560 nm (preferably 518-545 nm, most preferably 525-540 nm or approximately 530 nm).

In another aspect, the present disclosure provides a method of screening for a nuclease inhibitor, comprising: (a) contacting a candidate compound with a DNase and a fluorescent probe in a reaction mixture; (b) measuring DNase activity using the method of assaying DNase activity described above; and (c) identifying the candidate compound is a nuclease inhibitor if the DNase activity is reduced by a specified threshold compared to a control.

In some embodiments, the specified threshold is at least 30%, 40%, 50%, 60%, 70%, preferably at least 50%. In some embodiments, the method for nuclease inhibitor screening further comprises (c) calculating an IC₅₀ value for the candidate compound. In some embodiments, the DNase is a recombinant human DNASE1L3. In some embodiments, the method for nuclease inhibitor screening is performed in a high-throughput format using a multi-well plate.

In another aspect, the present disclosure provides a kit for carrying out the methods described herein. In some embodiments, the kit comprises (a) a DNase; and (b) a fluorescent probe. In some embodiments, the DNase is a recombinant human DNASE1L3. In some embodiments, the fluorescent probe comprises a single stranded DNA oligonucleotide linked with a fluorophore at the 5 terminus and a quencher at the 3 terminus, and wherein the fluorescent probe does not comprise an internal quencher. In some embodiments, the fluorophore is FAM and the quencher is IABkFQ. In some embodiments, the kit further comprises a reaction buffer comprising divalent cations Ca²⁺ and Mg²⁺.

In another aspect, the present disclosure provides a fluorescent probe for assaying DNASE1L3 activity, comprising a single-stranded DNA oligonucleotide linked with a fluorophore at the 5 terminus and a quencher at the 3 terminus. In some embodiments, the single-stranded DNA oligonucleotide does not comprise an internal quencher. In some embodiments, the fluorophore is FAM and/or the quencher is IABkFQ.

In another aspect, the present disclosure provides a method of calibrating DNASE1L3 activity. In particular embodiment, the method of calibrating DNASE1L3 activity comprises (a) incubating DNASE1L3 with the probe described herein over a range of probe concentrations from 10 nM to 100 nM, or over a range of DNASE1L3 concentrations from 10 pM to 2000 pM; (b) measuring fluorescence intensity over time at 37° C.; and (c) selecting a working probe concentration or a working DNASE1L3 concentration where fluorescence increase is linear with respect to the range of probe concentrations or the range of DNASE1L3 concentrations, thereby calibrating DNASE1L3 activity.

In another aspect, the present disclosure provides a method of screening a nuclease inhibitor using a calibrated DNASE1L3 through the method of calibrating DNASE1L3 activity described above. In particular embodiment, the method of screening a nuclease inhibitor using a calibrated DNASE1L3 comprises (a) contacting a candidate with the calibrated DNASE1L3 and the probe at the selected working concentrations; (b) measuring residual DNASE1L3 activity; and (c) identifying the candidate as a nuclease inhibitor if the residual DNASE1L3 activity is reduced by at least 50% relative to a no-candidate control.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows active site configuration of DNASE1 and DNASE1L3 Protein Data Bank (PDB) identification code (PDBID). PDBID 2DNJ depicts bovine DNASE1-induced DNA conformation; 1DNK depicts the crystal structure of a complex between DNASE1 and the self-complementary octamer duplex d (GGTATACC) 2 PDBID; 7KIU depicts the structure of recombinant human DNASE1L3 in a complex with Mg²⁺. The overlapping structure of DNASE1 and DNASE1L3 shows the two proteins share a similar structure including key residues maintaining activity.

FIGS. 2A-2C show an overview of Interformer model architecture. FIG. 2A depicts graph representation in either ligand or protein pocket format; FIG. 2B depicts a docking pipeline; and FIG. 2C depicts a PoseScore and Affinity prediction pipeline.

FIGS. 3A-3B show non-covalent interactions between ligand and protein atoms modeled by different approaches. FIG. 3A depicts an incorrect case generated by DiffDock (Corso et al. arXiv preprint arXiv. 2022; 2210:01776). Black and pattern arrows indicate improperly formed hydrogen bond and hydrophobic interactions (RMSD: 1.13, PDB ID: 6qmt). FIG. 3B depicts a correct binding pose generated by Interformer, where the predicted interaction energy function recovers almost all hydrogen bonds and hydrophobic interactions (RMSD: 0.67).

FIG. 4 illustrates molecular interactions between DNASE1 and Ceforanide to explain the structure-activity relationship (SAR). The illustration of DNASE1-Drug complex is predicted using Interformer based on the structure of PDB ID: 2DNJ.

FIG. 5 illustrates molecular interactions between DNASE1L3 and Ceforanide to explain the structure-activity relationship (SAR). The illustration of DNASE1L3-Drug complex is predicted using a diffusion-based docking based on the structure of PDB ID: 7KIU.

FIG. 6 shows a computational drug-screening model combining a DiffDock pose ranking and a Simina binding affinity score.

FIG. 7 illustrates prediction of antibiotics-Dnase affinity using the computational drug-screening model. The X axis represents drug affinities to DNase1L3, and the Y axis represents drug affinities to DNase1, with higher affinity being more negative in kcal/mol.

FIGS. 8A-8B show a correlation between fluorescent signals and probe concentrations.

FIG. 8A shows time courses of fluorescence intensity changes at various probe concentrations. Assays were performed in a solution (20 mM Tris-HCl, pH 7.5, 2 mM MgCl₂, 2 mM CaCl₂), 0.01% v/v Tween 20) in the presence of 20 pM DNASE1L3. Fluorescence intensities were measured at 530 nm upon excitation at 468 nm with a constant temperature at 37° C. FIG. 8B shows a linear relationship between the probe concentrations and fluorescence intensities. A linearity curve was generated for each probe concentration at 30 minutes, showing that there is a linear relationship between probe concentrations and fluorescence intensities without saturation.

FIGS. 9A-9B show a correlation between fluorescent signals and enzyme concentrations. FIG. 9A shows time courses of fluorescence intensity changes in response to different concentrations of enzyme DNASE1L3. Various concentrations of enzyme were tested for the assay. Reactions were performed in a solution (20 mM Tris-HCl, pH 7.5, 2 mM MgCl₂, 2 mM CaCl₂), 0.01% v/v Tween 20, 50 nM probe). Fluorescence intensities were measured at 530 nm upon excitation at 468 nm with a constant temperature at 37° C. FIG. 9B shows a linear relationship between the enzyme concentrations and fluorescence intensities. A linearity curve was plotted with fluorescence intensities at 30 minutes against all concentrations of enzyme. As the linearity shown, 1280 pM was the concentration of the enzyme chosen where the fluorescence intensity varies most with the largest range.

FIG. 10 shows a schematic of the experimental workflow for testing the inhibition of DNASE1 or DNASE1L3 by a drug compound using a fluorescent probe as the substrate.

FIGS. 11A-11B show an inhibitory effect of the drug compound ceforanide on the enzyme DNASE1. FIG. 11A depicts independent replicates of various concentrations of the enzyme were tested for the assay. FIG. 11B depicts dose-response curves of ceforanide inhibition on DNASE1. Based on the plot, IC50 was determined as 4353 μM. The mean is shown with error bars and dotted lines representing the standard deviation (SD) at each concentration and the 95% confidence interval (CI), respectively. Error bars show the SD of three independent experiments.

FIGS. 12A-12B show the inhibitory effect of the drug compound ceforanide on the enzyme DNASE1L3. FIG. 12A depicts independent replicates of various concentrations of the enzyme were tested for the assay. FIG. 12B depicts dose-response curves of ceforanide inhibition on DNASE1L3. Based on the plot, IC50 was determined as 18.94 μM. Error bars show the SD of three independent experiments.

FIGS. 13A-13B show the inhibitory effect of the drug compound cefuroxime sodium on the enzyme DNASE1L3. FIG. 13A depicts independent replicates of various concentrations of the enzyme were tested for the assay. FIG. 13B depicts dose-response curves of cefuroxime sodium inhibition on DNASE1L3. Based on the plot, IC50 was determined as 1352 μM. Error bars show the SD of three independent experiments.

FIG. 14A-14B show the inhibitory effect of the drug compounds minocycline and demeclocycline on the enzyme Human DNASE1L3. FIG. 14A depicts dose-response curve of minocycline inhibition on human DNASE1L3. Based on the plot, IC50 of minocycline was determined as 77 μM. FIG. 14B depicts dose-response curve of demeclocycline inhibition on human DNASE1L3. Based on the plot, IC50 of demeclocycline was determined as 105 μM. Error bars show the SD of three independent experiments.

FIG. 15 shows a schematic of a large-scale fluorescent-based drug screening platform.

FIG. 16. depicts a schematic workflow of the Baculovirus-insect cell recombinant protein expression system for human DNASE1L3.

FIGS. 17A-17B show the purification and function analysis of recombinant human DNASE1L3 from baculovirus-insect cell expression system. FIG. 17A depicts a purified recombinant protein was analyzed by SDS-PAGE and Western blot to confirm its identity. FIG. 17B depicts an activity test of recombinant human DNASE1L3.

FIG. 18 is a flowchart illustrating a method 1800 of assaying cell-free DNA (cfDNA) from a bodily fluid of a subject according to embodiments of the present invention.

FIG. 19 is a flowchart illustrating a method 1900 of inhibiting activity of a nuclease in a subject according to embodiments of the present invention.

FIG. 20 shows a measurement system 2000 according to embodiments of the present disclosure.

FIG. 21 shows a block diagram of an example of system usable with systems and methods according to embodiments of the present disclosure.

### TERMS

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, the term “optional” or “optionally” means that the event or situation described subsequently may occur but does not have to occur.

In the present application, the term “comprise” generally refers to the meaning of including, inclusive, containing, or encompassing. In some cases, it also means “is/are” and “consist of”.

The term “cell-free DNA” or “cfDNA” refers to deoxyribonucleic acid fragments that occur extracellularly. In some embodiments, cfDNA are degraded DNA fragments released to body fluids such as blood plasma, urine, cerebrospinal fluid. The term cfDNA can be used to describe various forms of DNA freely circulating in body fluids, including circulating tumor DNA (ctDNA), cell-free mitochondrial DNA (ccf mtDNA), cell-free fetal DNA (cffDNA) and donor-derived cell-free DNA (dd-cfDNA). Cell-free DNA may interact with other species, such as histone proteins, to form higher order structures, such as nucleosomes. In some embodiments, cell-free DNA originates from the cells of a subject. In some embodiments, cell-free DNA originates from both healthy and diseased cells of a subject. In some embodiments, cell-free DNA encodes one or more genes belonging to the subject's genome.

In some embodiments, cell-free DNA contains one or more mutations that are indicative of a disease, such as cancer. The term “mutation” as may be used herein, refers to a change, alteration, or modification to a nucleotide in a nucleic acid (e.g., a cfDNA) as compared to its wild-type sequence. For example, without limitation, mutations may include substitutions, insertions, deletions, or any combination of the same in the cfDNA which is concentrated and then assayed by the methods described herein.

In some embodiments, the mutations are “disease-associated” mutations, which refers to mutations that predict that the subject in which the mutation exists has an increased chance or risk of having or developing a disease, e.g., cancer. Disease-associated mutations may also include mutations that change the copy number of a gene by either duplicating or removing all or part of a gene in the genome. Disease-associated mutations may also include epigenetic modifications, such as changes in DNA methylation. DNA methylation refers to the addition of a methyl group to certain bases (C or A) in a nucleic acid molecule (e.g., changing C to 5-methylcytosine, mC). Changes in DNA methylation can occur within a coding portion of a gene (part of a gene that is transcribed and translated into protein) or within a non-coding portion of a gene (part of a gene that is transcribed and not translated into protein). Changes in DNA methylation can occur within the promotor sequence of a gene. Changes in the DNA methylation pattern of a gene can its expression level (i.e., cause it to be expressed at a higher or lower level than it is typically expressed).

The terms “increase”, “enrich”, “enhance” and “improve” are all generally statistically significant herein. The terms are used to indicate an increase in quantity, concentration, level, or the like, of cfDNA in a biological sample, such as a bodily fluid sample in certain volume. In some embodiments, the term “increase”, “enrich”, “enhance” or “improve” indicates to increase at least 10%, at least about 20%, or at least about 30%, or at least about 40%, or at least about 50%, or at least about 60%, or at least about 70%, or at least about 80%, or at least about 90%, or up to 100% increase compared to a reference level. The increase level may also be expressed in terms of fold-increase. In some embodiments, the term “increase”, “enrich”, “enhance” or “improve” indicates at least a 2-fold increase, or at least a 3-fold increase, or at least a 4-fold increase, or at least a 5-fold increase, or at least a 6-fold increase, or at least a 7-fold increase, or at least a 8-fold increase, or at least a 9-fold increase, or at least a 10-fold increase, or more, compared to a reference level.

The terms “decrease”, “reduce”, “reduction”, and “inhibit” are all generally statistically significant herein. The terms are used to indicate a decrease in quantity, concentration, function level, activity level, or the like, of nuclease (e.g., DNase1 or DNase1L3) in a biological sample, such as a bodily fluid sample in certain volume. In some embodiments, the term “decrease”, “reduce”, “reduction”, or “inhibit” indicates a reduction of at least 10%, or at least about 20%, or at least about 30%, or at least about 40%, or at least about 50%, or at least about 60%, or at least about 70%, or at least about 80%, or at least about 90%, or up to 100% reduction compared to a reference level. The reduction level may also be expressed in terms of fold-reduction. In some embodiments, the term “decrease”, “reduce”, “reduction”, or “inhibit” indicates at least a 2-fold reduction, or at least a 3-fold reduction, or at least a 4-fold reduction, or at least a 5-fold reduction, or at least a 6-fold reduction, or at least a 7-fold reduction, or at least a 8-fold reduction, or at least a 9-fold reduction, or at least a 10-fold reduction, or more, compared to a reference level.

The term “biological sample” as used herein generally refers to a tissue or bodily fluid sample derived from a subject. Biological samples can be obtained directly from a subject. The biological sample can be or can comprise one or more nucleic acid molecules, such as deoxyribonucleic acid (DNA) or ribonucleic acid (RNA) molecules. The biological sample can be taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia). The biological sample may be derived from any organ, tissue or biological fluid. The biological sample may comprise, for example, bodily fluids or solid tissue samples. An example of a solid tissue sample is a tumor sample, e.g., a solid tumor biopsy. Bodily fluids (or body fluids) include, for example, blood, plasma, serum, urine, vaginal fluid, fluid from a hydrocele (e.g., of the testis), vaginal flushing fluids, pleural fluid, ascitic fluid, cerebrospinal fluid (CSF), saliva, sweat, tears, sputum, bronchoalveolar lavage fluid, peritoneal fluid, discharge fluid from the nipple, aspiration fluid from different parts of the body (e.g., thyroid, breast), intraocular fluids (e.g., the aqueous humor), amniotic fluid, aqueous humor, ascites, bone marrow fluid, lymphatic fluid, synovial fluid, interstitial fluid, prostate fluid, semen, mucus, gastric acid, bile, pus, cerumen, breast milk, cowper's fluid or pre-ejaculatory fluid, female ejaculate, hair oil, cyst fluid, dialysis fluid, pericardial fluid, chyme, chyle, menses, sebum, vomit, mucosal secretion, stool water, pancreatic juice, lavage fluids from sinus cavities, bronchopulmonary aspirates, blastocoel fluid, urinary tract secretions, urethral secretions, bladder secretions, prostate secretions, vesical secretions, meconium, and umbilical cord fluid, etc. Stool samples can also be used.

In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The term “liquid biopsy” as used herein generally refers to a non-invasive or minimally invasive laboratory test or assay of a liquid biological sample (e.g. a bodily fluid sample). Such a “liquid biopsy” assay may report a measurement of one or more tumor-associated marker genes (e.g., minor allele frequency, gene expression, or protein expression). For example, a circular tumor DNA test from Guardant Health, a Spotlight 59 oncology panel from Fluxion Biosciences, an Ultrasik from Agena Bioscience, such as the UltraSEEK lung cancer panel, the Foundation ACT fluid biopsy assay from Foundation Medicine, and the PlasmaSELECT assay from Personal Genome Diagnostics, are commercially available. Such assays may report a measure of the minor allele fraction (MAF) value for each set of genetic variants (e.g., single nucleotide variation (SNV), copy number variation (CNV), insertion/deletion (Indel), and/or fusion). The methods and compositions described herein for boosting the levels of cfDNA may be used in combination with a liquid biopsy to assay for the presence of a disease marker. In some embodiments, the liquid biopsy involves a non-invasive or minimally invasive laboratory test or assay on a sample of blood. In such cases, the liquid biopsy can be referred to as a “blood biopsy”.

The terms “subject,” “individual,” and “patient” are used interchangeably herein to refer to a vertebrate, preferably a mammal, more preferably a human. The mammal can be a human, non-human primate, mouse, rat, dog, cat, horse, or cow, but is not limited to these examples. Mammals other than humans can be conveniently used, for example, as subjects that represent animal models of cancer, e.g., a particular type of cancer, such as, lung cancer. The subject can be male or female. In various embodiments, the subject has or is at risk of having a disease state, such as cancer, and is in need of being evaluated, e.g., by a liquid biopsy, to test for the risk of having or developing a disease, e.g., cancer. In other embodiments, the subject has already been diagnosed or identified as having or being at risk of having a disease in need of treatment (e.g., cancer), or one or more complications associated with such diseases. In other embodiments, a subject has already been treated for a disease (e.g., cancer) or one or more complications associated with a disease, such as cancer. Alternatively, a subject can also be a subject that has not been previously diagnosed as having a disease (e.g., cancer) or one or more complications associated with the disease. For example, a subject can be a subject that exhibits one or more risk factors for a disease, or one or more complications associated with the disease (e.g., cancer), or a subject that does not exhibit a risk factor. As disclosed herein, a subject who has a condition, has been diagnosed with a condition, or is at risk of developing the condition can be referred as a “subject in need” of a diagnosis and/or treatment for the particular condition (e.g., cancer).

As used herein, the term “administering” includes oral administration, topical contact, administration as a suppository, intravenous, intraperitoneal, intramuscular, intralesional, intratumoral, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “therapeutically effective amount” refers to the amount of an agent (e.g., a nuclease inhibitor described herein) that is required to bring about an effect in a subject when administered to the subject. As used herein, a therapeutically effective amount does not require a clinically effective amount. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the dosing regimen to be followed, whether it is administered in combination with other compounds, timing of administration, and the physical delivery system in which it is carried.

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

The term “Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

The term “recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide). A recombinant cell is one that has been modified (e.g., transfected or transformed), with a recombinant nucleotide, expression vector or cassette, or the like.

The term “polynucleotide” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. A polynucleotide includes nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location. Various alignment tools can be used, such as BLAST, BLASTZ, FASTA, G-PAS, SSEARCH, BOWTIE, AMAP, or SOAP.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine where a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

A “pocket site”, also known as a binding pocket, is a three-dimensional concave area or cavity found in the molecular structure of a protein or other biomolecule. It is distinguished by particular physiochemical and spatial characteristics that enable non-covalent interactions with ligands, substrates, or small molecules. Through a variety of intermolecular forces, such as hydrogen bonding, van der Waals interactions, electrostatic interactions, and hydrophobic effects, the cavity's defined volume, surface topography, and complementary chemical features allow for selective molecular recognition and binding. A key factor in biological processes and therapeutic interventions, the pocket's structural arrangement and amino acid composition dictate its binding specificity and affinity for specific molecular entities.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 15, 20, 30, 40, 50 60, 64, 70, 80, 90, 100, 150, 200, 250, or 256 end motifs. Further details about end motifs can be found in U.S. Patent Publications 2020/0199656, 2022/0010353, 2023/0313314, and 2024/0043935.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using the 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

An “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range). A “reference end-motif profile” or an “F-profile” refers to an end-motif profile that can be generated by applying a factorization algorithm (e.g., non-negative matrix factorization) to relative frequencies of DNA molecules of a given biological sample across a plurality of end motifs (e.g., 256 end motifs). Further details about end motif profiles can be found in U.S. Patent Publication 2024/0182982.

“Single-molecule sequencing” refers to sequencing of a single template DNA molecule to obtain a sequence read without the need to interpret base sequence information from clonal copies of a template DNA molecule. The single-molecule sequencing may sequence the entire molecule or only part of the DNA molecule. A majority of the DNA molecule may be sequenced, e.g., greater than 50%, 55%, 60%, 65%, 70%, 75%, 80%, 85%, 90%, 95%, or 99%. A sequence read (or reads from both ends) can be aligned to a reference genome. When both ends are aligned (e.g., as part of a read of the entire fragment or for paired-ends), greater accuracy can be achieved in the alignment and a length of the fragment can be obtained. Embodiments of the present disclosure can use single-molecule sequencing.

The term “alleles” refers to alternative DNA sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically include multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, sequence variants/mutations (which may be disease causing) and copy number variations (also referred to as a copy number aberration). The term “haplotype” can refer to a combination of alleles or epigenetic markers (e.g., methylation) at multiple loci that are transmitted together on the same chromosome or chromosomal region. A haplotype may refer to as few as one pair of loci or to a chromosomal region, or to an entire chromosome or chromosome arm.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range. Other parameters can include an average, median, mode, or mean. Further details about size profiles can be found in U.S. Patent Publications 2011/0276277, 2013/0040824, 2016/0201142, 2016/0217251, 2018/0208999, 2019/0130065, and 2019/0341127.

“DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported. Further details about methylation can be found in U.S. Patent Publications 2018/0216191, 2016/0017419, 2017/0029900, and 2019/0032145.

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a DNA fragment or whether a particular site in a genome has a particular differential methylation status, e.g., hypermethylation or hypomethylation. A “read” can include information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110:18910-18915) and by the Pacific Biosciences single molecule real time analysis (Tse et al. Proc Natl Acad Sci USA 2021; 118: e2019768118).

A “methylation level” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules at one or more sites can be determined. The relative abundance can also include an intensity per volume. A methylation level can be determined using a methylation-aware assay such as methylation-aware sequencing or PCR. Example methylation-aware sequencing can include bisulfite sequencing or single molecule techniques, e.g., using nanopores.

The term “damage” when describing DNA molecules may refer to DNA nicks, single strands present in double-stranded DNA, overhangs of double-stranded DNA, oxidative DNA modification with oxidized guanines, abasic sites, thymidine dimers, oxidized pyrimidines, blocked 3′ end, or a jagged end.

The term “jagged end” may refer to sticky ends of DNA, overhangs of DNA, or where a double-stranded DNA includes a strand of DNA not hybridized to the other strand of DNA. “Jagged end value” or “jagged index” is a measure of the extent of a jagged end. The jagged end value may be correlated (e.g., proportional) to an average length of one strand that overhangs a second strand in double-stranded DNA. The jagged end value of a plurality of DNA molecules may include consideration of blunt ends among the DNA molecules.

In some instances, the jagged index value can provide a collective measure that a strand overhangs another strand in a plurality of cell-free DNA molecules. The collective measure of jaggedness can be determined based on an estimated length of overhang in the plurality of cell-free DNA molecules, e.g., an average, median, or other collective measure of individual measurements of each of the cell-free DNA molecules. In some instances, the collective measure of jaggedness is determined for a particular fragment size range (e.g., 130-160 bps, 200-300 bps). In some instances, the collective measure of jaggedness can be determined based on the methylation signal changes proximal to the ends of the plurality of cell-free DNA molecules.

A “relative frequency” (also referred to just as “frequency”) may refer to a proportion (e.g., a percentage, fraction, or concentration). In particular, a relative frequency of a particular end motif (e.g., A, CG, TAG, etc.) or end motif pair (e.g., A< >A) can provide a proportion of cell-free DNA fragments that have that end motif or that particular pair end motif pair.

An “aggregate value” may refer to a collective property, e.g., of relative frequencies of a set of end motifs. Examples include a mean, a median, a sum of relative frequencies, a variation among the relative frequencies (e.g., entropy, standard deviation (SD), the coefficient of variation (CV), interquartile range (IQR) or a certain percentile cutoff (e.g., 95th or 99th percentile) among different relative frequencies), or a difference (e.g., a distance) from a reference pattern of relative frequencies, as may be implemented in clustering. As another example, an aggregate value can comprise an array/vector of relative frequencies, which can be compared to a reference vector (e.g., representing a multidimensional data point).

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” (e.g., an amount of fragments with a particular end motif or with a particular size) and a measured or known value that is desired to be determined for other test samples (e.g., a fractional concentration of the clinically-relevant DNA such as DNA of particular tissue type). The calibration value can be determined from various types of data measured from DNA molecules of the sample, (e.g., an amount of fragments with an end motif or with a particular size, such as relative frequencies (e.g., an aggregate value) as determined for a calibration sample). The calibration value corresponds to a parameter that correlates to the desired property, e.g., classification of a genetic disorder, nuclease activity, or efficacy of anticoagulant dosage. For example, a calibration value can be determined from measured values as determined for a calibration sample, for which the desired property is known. The measured or known value (e.g., a fractional concentration) can be determined in various ways, e.g., using a tissue-specific allele, a tissue-specific methylation value or pattern, and a size distribution of a sample with a known fractional concentration. The calibration data points may be defined in a variety of ways, e.g., as discrete points or as a calibration function (also called a calibration curve or calibration surface). The calibration function could be derived from additional transformation of the calibration data points.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein can refer to a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter. A normalized amount can account for a size of a sample, e.g., volume, mass, or number of nucleic acid molecules analyzed.

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (In) of the two values. A separation value can include a difference and a ratio. A separation value can be compared to a threshold to determine whether the separation between the two values is statistically significant.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The term “sequence imbalance” or “aberration” or “copy number aberration (CNA)” as used herein means any significant deviation as defined by at least one cutoff value in a quantity of the clinically relevant chromosomal region from a reference quantity. A sequence imbalance can include chromosome dosage imbalance, allelic imbalance, mutation dosage imbalance, copy number imbalance, haplotype dosage imbalance, and other similar imbalances. As an example, an allelic imbalance can occur when a tumor has one allele of a gene deleted or one allele of a gene amplified or differential amplification of the two alleles in its genome, thereby creating an imbalance at a particular locus in the sample. As another example, a patient could have an inherited mutation in a tumor suppressor gene. The patient could then go on to develop a tumor in which the non-mutated allele of the tumor suppressor gene is deleted. Thus, within the tumor, there is mutation dosage imbalance. When the tumor releases its DNA into the plasma of the patient, the tumor DNA will be mixed in with the constitutional DNA (from normal cells) of the patient in the plasma. An aberration can include a deletion or amplification of a chromosomal region.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The terms “cancer” or “tumor” may be used interchangeably and generally refer to an abnormal mass of tissue wherein the growth of the mass surpasses and is not coordinated with the growth of normal tissue. A cancer or tumor may be defined as “benign” or “malignant” depending on the following characteristics: degree of cellular differentiation including morphology and functionality, rate of growth, local invasion, and metastasis. A “benign” tumor is generally well differentiated, has characteristically slower growth than a malignant tumor, and remains localized to the site of origin. In addition, a benign tumor does not have the capacity to infiltrate, invade, or metastasize to distant sites. A “malignant” tumor is generally poorly differentiated (anaplasia), has characteristically rapid growth accompanied by progressive infiltration, invasion, and destruction of the surrounding tissue. Furthermore, a malignant tumor has the capacity to metastasize to distant sites. “Stage” can be used to describe how advance a malignant tumor is. Early stage cancer or malignancy is associated with less tumor burden in the body, generally with less symptoms, with better prognosis, and with better treatment outcome than a late stage malignancy. Late or advanced stage cancer or malignancy is often associated with distant metastases and/or lymphatic spread.

The term “level of cancer” can refer to whether cancer exists (i.e., presence or absence), a stage of cancer, a size of tumor, whether there is metastasis, the total tumor burden of the body, the cancer's response to treatment, and/or other measure of a severity of cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, one million, ten million, 100 million, or one billion parameters. An ML model can be generated using sample data (e.g., training samples) to make predictions on test data. Various number of training samples can be used, e.g., at least 10, 100, 1,000, 5,000, 10,000, 50,000, 100,000, or 200,000 training samples. One example is reinforcement learning such as Q-Learning, Deep Q-Networks (DQN), Double DQN, Dueling DQN, Policy Gradient Methods, Actor-Critic, Advantage Actor-Critic (A2C), Proximal Policy Optimization (PPO), Trust Region Policy Optimization (TRPO), and Soft Actor-Critic (SAC). Another example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network (e.g. including convolutional and/or transformer layers) that may have 1-10 layers as examples, recurrent neural network (e.g., long short term memory, LSTM), boosting (meta-algorithm), bootstrap aggregating (bagging) such as random forests, support vector machine (SVM), support vector (SVR), Bayesian statistics, case-based reasoning, decision tree learning (e.g., CART (classification and regression trees), gradient boosted trees, or random forest), inductive logic programming, linear regression, logistic regression, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct (PAC) learning, ripple down rules, a knowledge acquisition methodology, symbolic machine learning algorithms, subsymbolic machine learning algorithms, minimum complexity machines (MCM), ordinal classification, data pre-processing, handling imbalanced datasets, statistical relational learning, or Proaftn (a multicriteria classification algorithm), or an ensemble of any of these types. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques. Some workflows may also include steps for data pre-processing or handling imbalanced datasets.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, preferably within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than a specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure. Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

The present disclosure provides methods, compositions, and systems that can improve the quantity and quality of cfDNA in a biological sample (e.g., a bodily fluid) of a subject. For example, the present disclosure provides a method of increasing cfDNA level in a bodily fluid of a subject, by administering a therapeutically effective amount of a nuclease inhibitor to the subject. The nuclease inhibitor inhibits cleavage of the cfDNA in the bodily fluid, thereby increasing the cfDNA level in the subject. This method is particularly useful for subjects who have been identified that a liquid biopsy assay is needed, such as patients at the early stages of cancer diagnosis, or pregnant subjects at risk of preeclampsia or other pregnancy-related complications, or cancer patients who have received cancer treatment and that periodic liquid biopsies are needed to monitor patients' conditions and adjust treatment.

The nuclease inhibitors disclosed herein can target various nucleases such as deoxyribonucleases (DNases), particularly deoxyribonuclease 1 (DNase1) and/or deoxyribonuclease 1-like 3 (DNase1L3), the two main deoxyribonucleases (DNases) associated with cfDNA fragmentation patterns in bodily fluids. In some embodiments, the nuclease inhibitor is an antibiotic such as a cephalosporin or sulfonamide. In particular embodiments, the nuclease inhibitor is the antibiotic ceforanide. Our study shows that ceforanide binds to a catalytic pocket of DNase1L3 and DNase1, and effectively reduces activities of DNase 1L3 and/or DNase1.

The present disclosure also provides compositions for increasing cfDNA level in a bodily fluid of a subject. In some embodiments, the compositions comprise one or more nuclease inhibitors described herein. Provided herein also includes a kit that comprises at least one reagent for carrying out the methods described herein.

The methods and compositions described herein can be used for cfDNA assessment.

Once the circulating cfDNA level is enriched in the bodily fluid of a subject by administering a therapeutically effective amount of a nuclease inhibitor (e.g. an antibiotic) to the subject, a bodily fluid sample can be collected for cfDNA assessment. Provided herein also include methods of characterizing biomarker(s) of interest from the enriched cfDNA samples, such as identifying copy numbers, sizes, end motifs, jagged ends, preferred ending positions, nucleosome footprint, etc. The biomarker(s) can be used to determine the subject and/or the sample as having a property, such as a disease (e.g. cancer) based on biomarker(s) of the cfDNA indicating the disease, a sequence imbalance, a particular fractional concentration of clinically-relevant DNA in the sample, and other properties mentioned herein. Desired treatments can be provided and/or administered against an identified disease.

The present disclosure provides methods of producing a recombinant human deoxyribonuclease (DNase) 1-like 3 (DNASE1L3). In addition, the present disclosure provides optimized in vitro assays for measuring DNASE1L3 activities and/or screening nuclease inhibitors, offering improved linearity, sensitivity, and scalability over conventional methods.

### I. Introduction

Cell-free DNA (cfDNA) plays an emerging role in various fields of modern medicine, including but not limited to, research, screening, diagnosis, and therapeutic management of different conditions (Lo et al. Science 2021; 372: eaaw3616; Lo et al. Lancet. 1997; 350:485-7). The past 20 years have witnessed remarkable progress in the clinical applications of liquid biopsy including but not limited to prenatal testing (Chiu et al. Proc Natl Acad Sci USA. 2008; 105:20458-20463; Chiu et al. BMJ. 2011; 342), oncology (Chan et al. N Engl J Med. 2017; 377:513-522; Heitzer et al. Nat Rev Genet. 2019; 20:71-88), infectious diseases (Blauwkamp et al. Nat Microbiol. 2019; 4:663-674; Wang et al. Clin Chem. 2023; 69:189-201), and organ transplant monitoring (Lo et al. Lancet. 1998; 351:1329-1330). The use of cfDNA plays a significant role in modern prenatal screening fetal chromosomal aneuploidies in a non-invasive manner. Via analysis of circulating cfDNA including circulating tumour DNA (ctDNA) in patients, one can derive a lot of information about the diseases in question. For example, inspecting genetic and epigenetic aberrations in plasma enabled the detection of patients with cancers across various stages as well as tumor localizations (Chan et al. Proc Natl Acad Sci USA. 2013; 110:18761-8; Sun et al. Proc Natl Acad Sci USA. 2015; 112: E5503-12).

However, the limited quantity of cfDNA in bodily fluids significantly restricts these applications. The quantity of cfDNA is governed by two factors: cfDNA production and cfDNA clearance. The cfDNA production is related to cell apoptosis and proliferation, and the cfDNA clearance is related to at least two biological processes: cfDNA uptake by the mononuclear phagocyte system (MPS) in the liver, and cfDNA digestion by nucleases in the blood. Using a mouse model, Martin-Alonso et al applied nanoparticles as competitors of circulating cfDNAs to be phagocytosed by macrophages (Martin-Alonso et al. Science. 2024; 383: eadf2341). Once the nanoparticles were administered to a level that the phagocytosis of the mononuclear phagocyte system was attenuated, the uptake of cfDNA was inhibited. On the other hand, Martin-Alonso et al used another way to elevate cfDNA levels using DNA-binding monoclonal antibodies (mAbs), which bind to cfDNAs and prevent their degradation from DNA nucleases. Considering the significant differences between mice and humans, an effective way to increase cfDNA levels in human subjects is still unclear. It was reported that the chromatin states and transcriptional activity were associated with the cfDNA presentation in blood circulation in humans (Che et al. Genome Res. 2024; 34:189-200). A significant divergence in sequences has been reported to influence transcriptional regulation, chromatin state, and higher-order chromatin organization between these two species (Yue et al. Nature. 2014; 515:355-364). This suggests that the mechanisms governing cfDNA clearance may differ substantially between mice and humans. Therefore, there is an urgent need to develop simple and efficient methods to increase cfDNA levels in human subjects.

To increase cfDNA levels in human subjects, one solution is to effectively inhibit the clearance of cfDNA mediated by nucleases in bodily fluids of the human subjects. Among many enzymes catalysing the hydrolysis of DNA, two main types of DNA nucleases, namely deoxyribonuclease 1 (DNASE1) and deoxyribonuclease 1-like 3 (DNASE1L3), are associated with cfDNA fragmentation patterns (Han et al. Am J Hum Genet. 2020; 106:202-214). It has been reported that G-actin can inhibit DNASE1 (Han, Lo, Trends Genet. 2021; 37:758-770), whilst 4-(4,6-dichloro-[1,3,5]-triazin-2-ylamino)-2-(6-hydroxy-3-oxo-3H-xanthen-9-yl)-benzoic acid (DR396), Pontacyl Violet 6R (PV6R), and Fmoc-D-Cha-OH (FDCO) can inhibit DNASE1L3 (Yamada et al. Bioorg Med Chem. 2011; 19:168-171; Kolarevic et al. Eur J Med Chem. 2014; 88:101-111). However, none of these inhibitors are FDA-approved, hence the clinical application of such chemical compounds in human subjects has not been achieved.

The present disclosure provides methods for screening new nuclease inhibitors that can efficiently reduce or block the nuclease activities. The candidate nuclease inhibitors are identified through analysing the interaction between one or more deoxyribonucleases and various proteins, nucleotides, antibiotics, and organic and inorganic compounds. In some instances, such interaction is analysed via machine learning approaches. In other instances, such interaction is analysed via molecular docking. In yet other instances, such interaction is analysed via molecular dynamics simulations. As DNASE1L3 and DNASE1 played a key role in cleaving cfDNA in blood circulation (Serpas et al. Proc Natl Acad Sci USA. 2019; 116:641-649), we are particularly interested in candidate nuclease inhibitors targeting to DNASE1L3 and/or DNASE1. In some embodiments, the nuclease inhibitors of interest are FDA-approved drugs.

In other embodiments, candidate compositions (e.g., drugs) can be identified using computational simulations, fluorescence-labeled probe-based enzyme activity screening, human/mouse plasma/serum chromatin digestion assay, in vivo mouse inhibition efficiency test (e.g. degree of cfDNA level elevation).

The present disclosure further provides methods and compositions for increasing cfDNA level in a bodily fluid (e.g., blood) of a subject. The methods described herein can block or reduce nuclease activities in the subject by introducing a therapeutically effective amount of a nuclease inhibitor to the subject, thereby accumulating the cfDNA of interest in the bodily fluid of the subject.

### II. In Silico Screening for Nuclease Inhibitors

Computational simulation (e.g. In silico docking studies) can be used to select and narrow down the candidate nuclease inhibitors of interest for experimental validation. For example, one could select candidate nuclease inhibitors that bind to the putative active sites of the enzymes on the basis of binding affinity, binding occupancy and binding coverage relative to putative active sites. Binding affinity is the measure of the strength of the interaction between a ligand, such as a drug, and its target, such as a protein or enzyme. The strength of the interaction is commonly measured using the equilibrium constant (Kd) or IC50 value, with a lower Kd or IC50 indicating a more powerful interaction. High binding affinity is advantageous in drug discovery because it indicates that a drug can efficiently inhibit its target at lower concentrations. Binding occupancy refers to the ratio of occupied binding sites on a target protein to the total number of binding sites, with respect to a specific concentration of a ligand. The influence on the system is determined by both the binding affinity of ligand and its concentration. A high binding occupancy indicates that a substantial proportion of the target binding sites are interacting with the ligands, which is crucial for the effectiveness of a drug in therapy. Binding coverage is the measure of how much of the binding sites on a target protein are occupied by ligands. The spatial arrangement of the ligand within the binding site and the existence of multiple binding conformations can have an impact on this. Significant binding coverage indicates that the ligand is effectively engaging with the target, potentially augmenting its biological activity.

In this disclosure, for illustration purposes, we first gathered 3,067 ligands from an FDA-approved drug library set by Selleck (Catalog No. L1300), with the basic chemical information and SMILES (Simplified Molecular Input Line Entry System). The term “ligand” refers to a molecule that binds to a central atom in a complex, e.g., that binds to a biomolecule to perform a biological function. SMILES is a specification in the form of a line notation for describing the structure of chemical species. For example, cyclohexane and dioxane may be written as C1CCCCC1 and O1CCOCC1 respectively. Secondly, we prepared these molecules by desalting, adding explicit hydrogen atoms, generating protonation states at pH 7.4, and varying chiral centers for atoms that do not have explicit chiral properties. We enumerated all possible ligand conformations, as a single molecule may exist in several potential states. This process resulted in a total of 7,831 ligand conformations. Thirdly, we prepared the protein conformation by adding explicit hydrogen atoms, assigning bond orders, generating protonation states at pH 7.4, optimizing the water network, and performing restrained energy minimization with a root mean square deviation (RMSD) cutoff at 0.3 Å. As disclosed herein, the protonation states can be generated at any pH value favorable to a water network. In some embodiments, the pH value is a pH value between 6.0 and 9.0, optionally a pH value between 7.0 and 8.0. A non-limiting example of an optimal pH value is 7.0, 7.1, 7.2, 7.3, 7.5, 7.6, 7.7, 7.8, 7.9, or 8. In some embodiments, the minimization of restrained energy is performed with a RMSD cutoff at 0.1 Å, 0.2 Å, 0.4 Å, 0.5 Å, 0.6 Å, or 0.7 Å.

To our knowledge, there are no crystal structures of native DNA nucleases or homologous proteins binding to a drug-like small molecule available. To make the in-silico analysis possible, we utilized PDBID 2DNJ as our initial crystal structure. This structure, derived from bovine DNASE1-induced DNA conformation, was released in 1994 and is a known DNASE1-related Protein Data Bank (PDB) (Lahm, Suck, J Mol Biol. 1991; 222:645-667). Using DNASE1 as an example, we aim to identity one or more ligands that could bind to the pocket site of DNASE1, thereby enabling the attenuation of its enzymatic cleavage ability. We hypothesized that the DNA strand cleavage occurred is related to the ligand pocket site of a DNA nuclease. We consider using the protein-ligand docking method to identify ligands that can bind to the target protein (e.g. DNA nuclease). This method operates under the assumption that the ligand pocket site is rigid. It establishes a cubic box with a 20 Å³ volume centered around the ligand pocket sites. The method then positions a given ligand within this box to find its optimal placement within the binding pocket by minimizing an energy function. The volume of hypothetic cubic box can be other values such as but not limited to 10 Å³, 15 Å³, 25 Å³, 30 Å³.

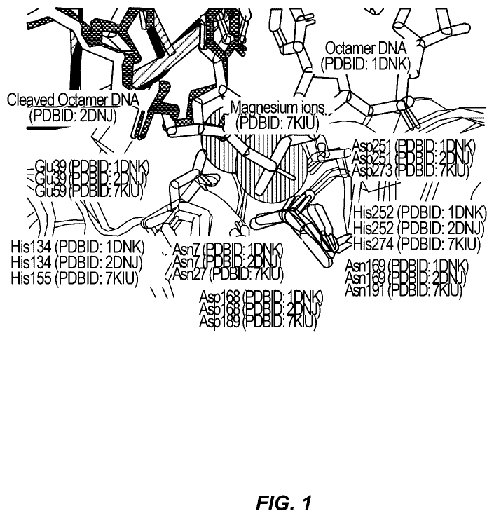

We performed docking for all ligand conformations related to this binding pocket and ranked them based on the affinity values predicted by the Interformer (Lai et al. doi.org/10.21203/rs.3.rs-3995849/v1, note: a preprinted paper) model. These values are expressed in terms of the negative logarithm of IC50 (the half-maximal inhibitory concentration), denoted as pIC50. A higher pIC50 value indicates greater affinity. The pIC50 can be converted to IC50 using the tenth power with a meter unit. The predicted pIC50 values range from 2.3 to 5.91, with a median of 4.0 across all generated docking poses. The top-ranked molecule was Ceforanide, with a predicted pIC50 value of 5.914, equivalent to an IC50 value of 1.218 μM (micromolar). pIC50 is a logarithmic scale utilized to quantify the effectiveness of a substance, such as a drug, in suppressing a particular biological or biochemical function. Ceforanide is classified as a second-generation cephalosporin, which disrupts the process of bacterial cell wall synthesis by inhibiting the penicillin-binding proteins (PBP), ultimately leading to bacterial cell lysis (Campoli-Richards et al. Drugs 1987; 34:411-437). FIG. 1B illustrates the chemical structure of ceforanide. We further subjected the top 100 ranked molecules to our internal MMGBSA (Molecular Mechanics, General Born Surface Area) pipeline to assess the binding free energy between the protein and ligand. The results showed a dG (Gibbs free energy change of binding, or ΔG) of −35.5, maintaining ceforanide in the top molecule rank.

We also explored the potential of ceforanide on DNASE1L3 using PDB ID 7KIU (McCord et al. Commun Biol. 2022; 5:825), a structure of recombinant human DNASE1L3 in a complex with Mg²⁺. We considered that Mg²⁺ likely only exists during interaction with DNA or in a free-circulating state. We removed Mg²⁺ from the complex to simulate rigid protein-ligand docking, but ceforanide did not rank in front. We hypothesized that the binding sites of a free-circulating DNase1L3 (7KIU) differ significantly from the binding sites of DNase1-induced DNA (2DNJ). This difference could hinder the smooth docking of ceforanide into the binding sites. Thereby, we employed induced fit docking with ceforanide to the 7KIU structure. This approach allows the adjustment of the side chain of the binding site to accommodate a given ligand during the docking process. As a result, we observed a binding pattern similar to the results obtained with 2DNJ, as shown in FIG. 1. Evaluation of this docking pose with Interformer resulted in a higher predicted pIC50 value of 6.5, equivalent to an IC50 of 0.31 μM. Consequently, we proceed with in vitro affinity experiments to validate the affinity of this ligand.

### A. Interformer: An Interactions-Aware Model for Protein-Ligand Docking and Affinity Prediction

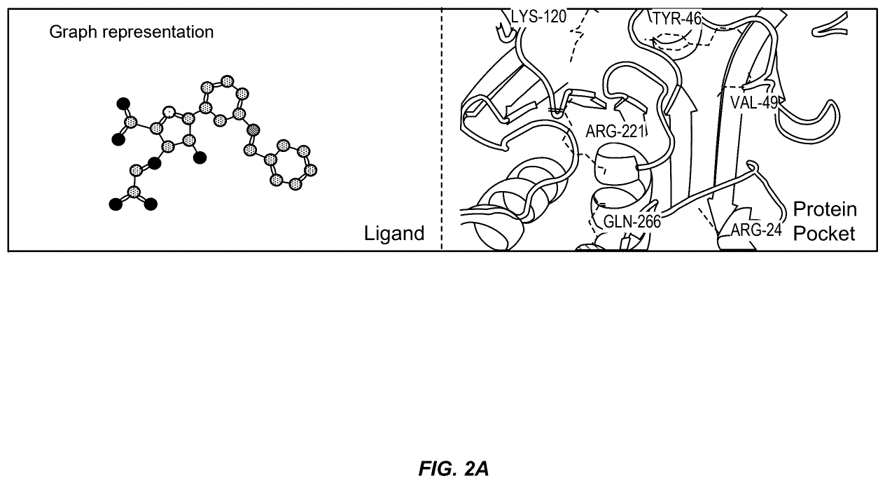

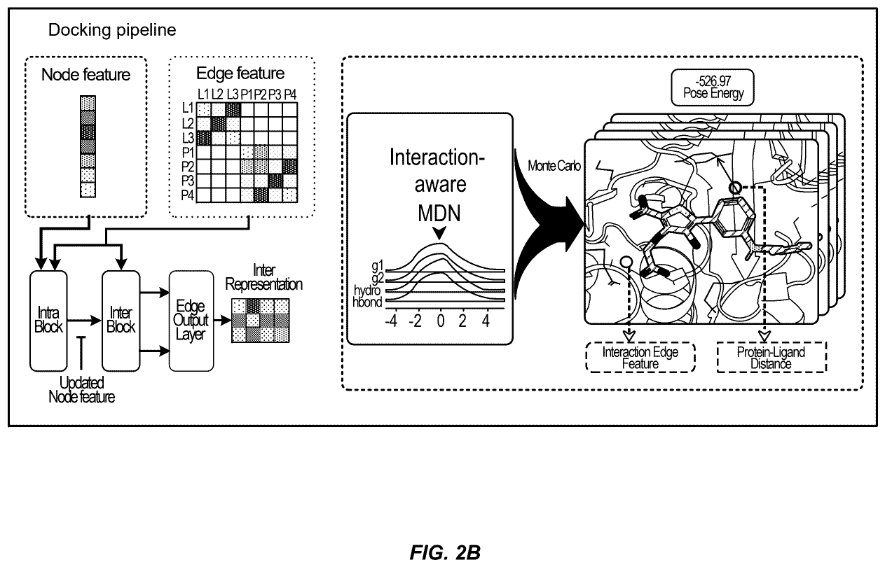

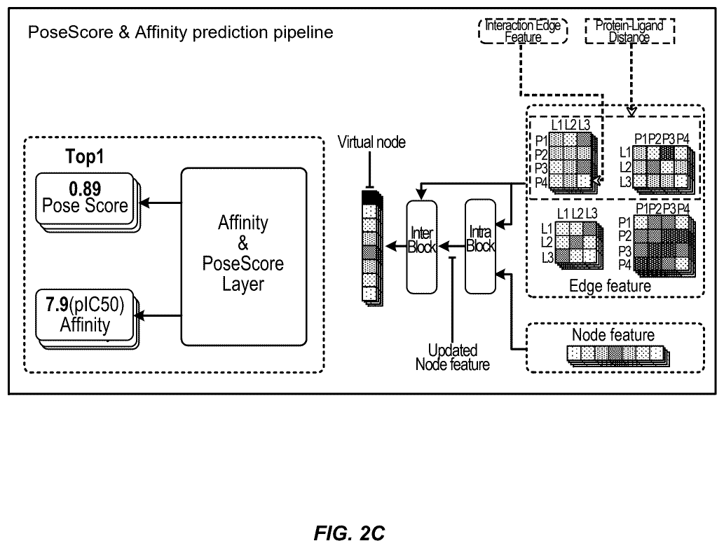

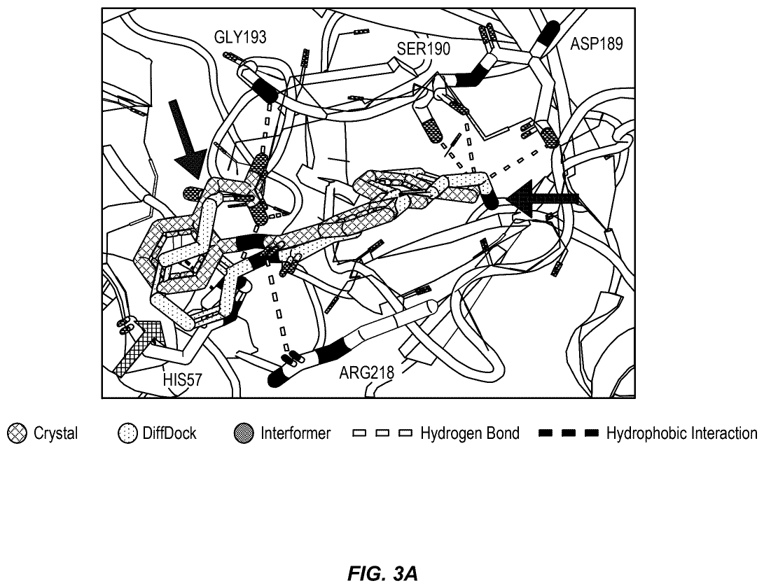

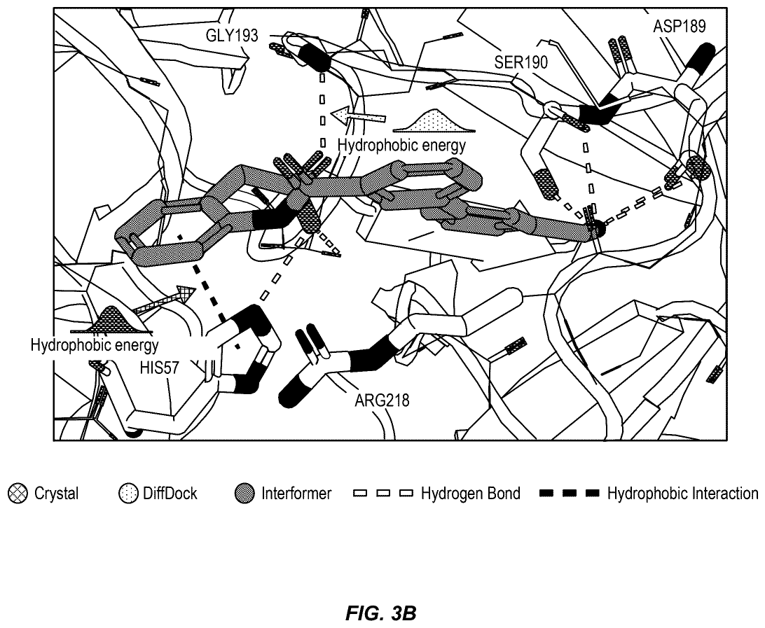

Interformer is an internally developed artificial intelligence (AI) model for protein and small molecule (ligand) docking and affinity prediction tasks, as shown in FIGS. 2A-2C. It is designed to capture non-covalent interactions by utilizing interaction-aware energy functions for docking (FIG. 2B). This energy function alleviates the issue prevalent in recent deep-learning models, which often overlook the modeling of non-covalent interactions between ligand and protein atoms, as shown in FIG. 3A. Specifically, this energy function emphasizes the modeling of hydrogen bonds and hydrophobic interactions, as shown in FIG. 3B. The Interformer can accurately generate specific interactions in the binding pose, thereby rendering the binding pose both plausible and rational. Furthermore, it employs a novel negative sampling strategy, enabling an effective correction of interaction distribution for affinity prediction, as shown in FIG. 2C. The Interformer accepts both initial ligand conformation and protein pocket conformation as inputs. It then docks the ligand to the pocket site using predicted energy functions combined with a Monte Carlo sampling method. Subsequently, the conformation of the docked protein-ligand complex is input into the affinity module implemented in Interformer to calculate their affinity value.

### B. Structure-Activity Relationship of Ceforanide on Interformer Predicted Docking Pose

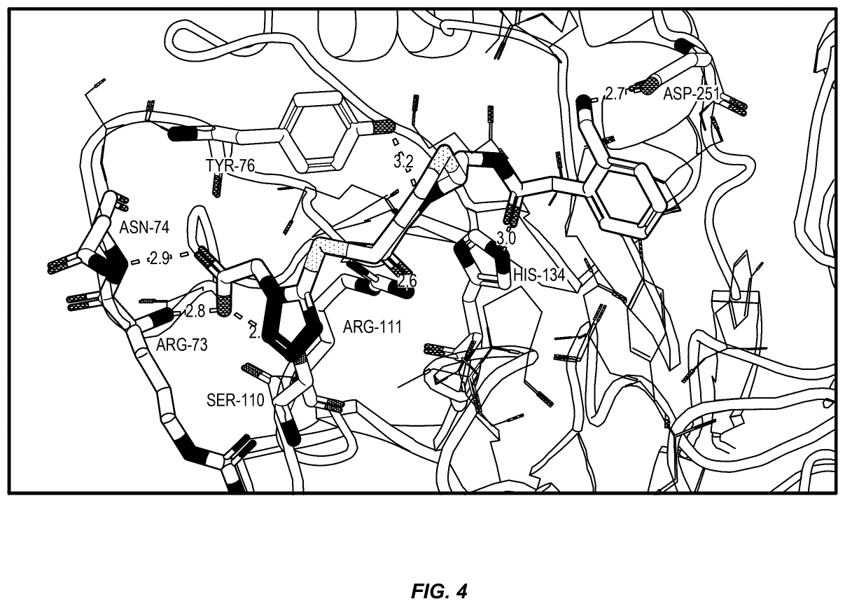

FIG. 4 illustrates the predicted DNASE1-Ceforanide complex based on 2DNJ, as determined by Interformer. These complex forms eight hydrogen bond interactions with the large hydrophobic groups of the benzene ring into the TYR211 residue; the ligand fits snugly within the pocket site, indicating strong non-covalent interactions between the protein and the ligand. As disclosed herein, Ceforanide binds to the TYR211 residue of DNASE1.

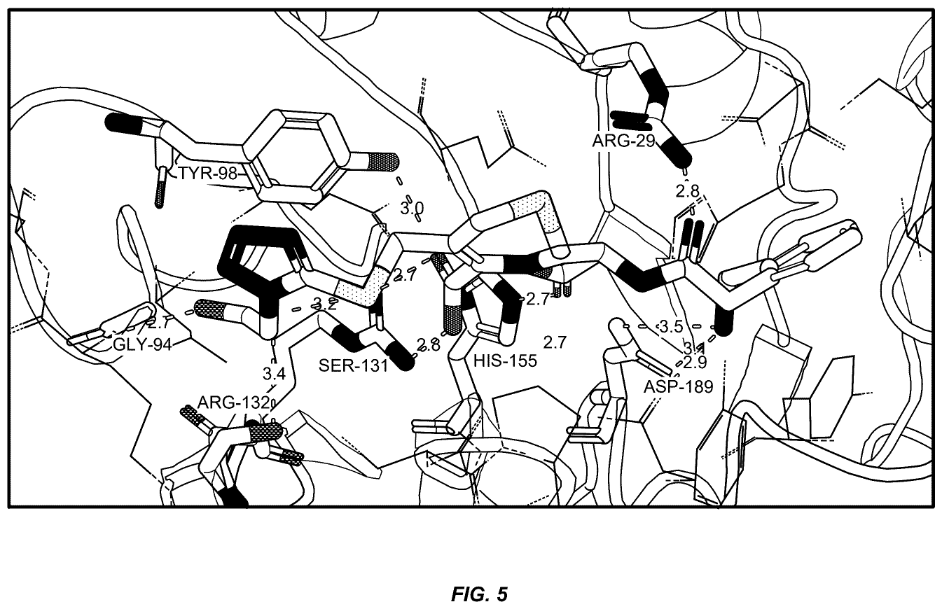

FIG. 5 illustrates the predicted DNASE1L3-Ceforanide complex, derived from induced fitting based on 7KIU. The interactions observed here are similar to those obtained with 2DNJ. However, an additional hydrogen bond is formed with the ARG29 residue, which points in the opposite direction in the original crystal structure. This observation implies that ARG29 might be attracted by Ceforanide, forming an extra hydrogen bond interaction and a cation-pi interaction with the benzene ring. It could explain why Ceforanide exhibits a stronger affinity for human DNASE1L3 than human DNASE1. We propose that ARG29 is a crucial residue that significantly influences binding affinity. As disclosed herein, Ceforanide binds to the ARG29 residue of DNASE1L3.

### C. Combining DiffDock Pose Ranking and Binding Affinity

To fully evaluate ligand-protein interactions across systems of varying sizes, we further developed a new workflow that combines DiffDock's AI-driven binding site prediction with Smina's affinity scoring function. This integration allows us to assess binding free energy, making it suitable for comprehensive analysis across different ligand-protein systems. Prediction of binding site by AI algorithm DiffDock plus estimation of binding free energy by Smina affinity scoring function allows comparison across different ligand-protein system. The model integrates two steps: \textcircled{1} DiffDock binding site prediction and \textcircled{2} Smina affinity score.

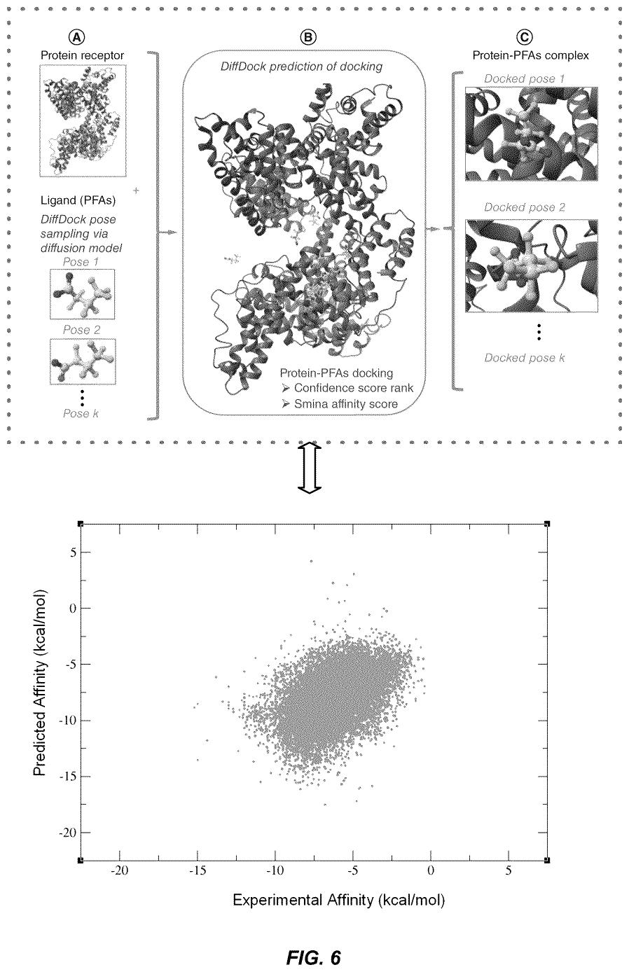

FIG. 6 illustrates a workflow that involves two key steps: 1. DiffDock Binding Site Prediction: Identifies potential binding sites using advanced AI algorithms as shown in the top part of FIG. 6. (B Fortela et al. (2024). BioTechniques, 76(1), 14-26), and 2. Smina Affinity Scoring: Estimates the binding free energy, providing valuable insights into interaction strength as shown in the bottom part of FIG. 6.

Initial results show promising correlations with a Pearson correlation of 0.452 and a Spearman correlation of 0.468, based on approximately 13,800 samples from the PDBBind dataset (unpublished). This demonstrates the potential of our approach, and we plan to explore additional docking workflows to further validate and enhance these findings.

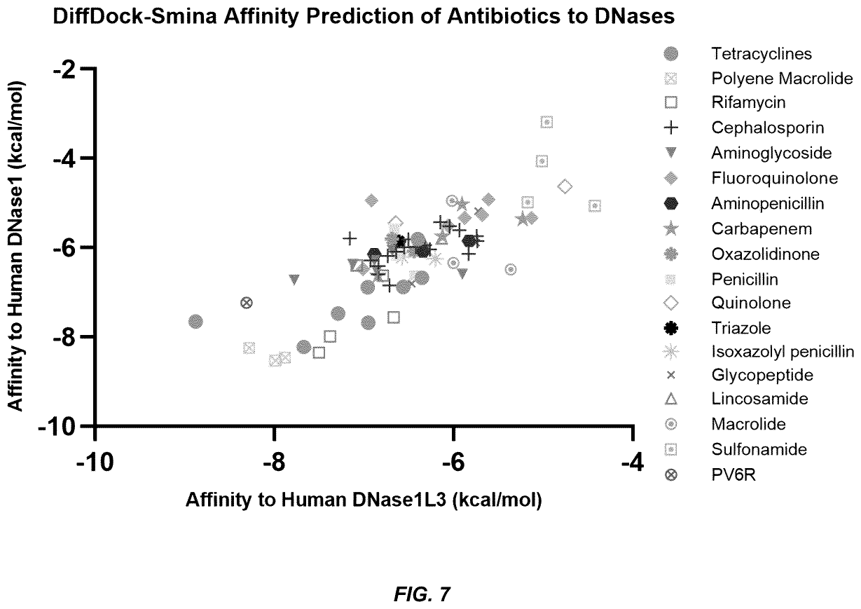

Using this model, we discovered several families of antibiotics with high affinities to DNase1L3 and Dnase1. As shown in FIG. 7, tetracycline, polyene macrolide, and rifamycin antibiotics displayed high affinities to both DNase1L3 and Dnase1. Table 1 shows tetracycline family and their affinities to DNase1L3 and Dnase1. Table 2 shows rifamycin family and their affinities to DNase1L3 and Dnase1.

TABLE 1
Tetracycline family

| | | | Affinity to |
|---|---|---|---|
| | | Affinity to | DNase1 |
| Tetracyclines | Route | DNase1L3(kcal/mol) | (kcal/mol) |
| Doxycycline | Oral, injection, | −8.879 | −7.657 |
| | periodontal | | |
| Demeclocycline | Oral | −7.671 | −8.229 |
| Tetracycline | Oral, ophthalmic, | −7.289 | −7.478 |
| | injection, topical, | | |
| | periodontal | | |
| Minocycline | Oral, injection, | −6.957 | −6.888 |
| | dental, topical | | |
| Omadacycline | Oral, intravenous | −6.953 | −7.682 |
| Sarecycline | Oral | −6.351 | −6.674 |

TABLE 2
Rifamycin family

| | | Affinity to | Affinity to |
|---|---|---|---|
| | | DNase1L3 | DNase1 |
| Rifamycin | Route | (kcal/mol) | (kcal/mol) |
| Rifaximin | Oral | −7.502 | −8.356 |
| Rifabutin | Oral | −7.378 | −7.987 |
| Rifamycin | Oral | −7.081 | −6.406 |
| Rifampin | Oral, injection | −6.787 | −6.627 |
| Rifapentine | Oral | −6.67 | −7.563 |

### III. In Vitro Results to Inhibit Nuclease Activity

The present disclosure provides exemplary in vitro experimental results evaluating the inhibition of nuclease activity by various compounds. We evaluated the methodologies and findings from the assessment of DNA nuclease activity and the inhibition profiles of several candidate inhibitors, including Ceforanide, Cefuroxime sodium, Minocycline, Demeclocycline, and Rifampin. The results provide important insights into the potency and specificity of these compounds as nuclease inhibitors.

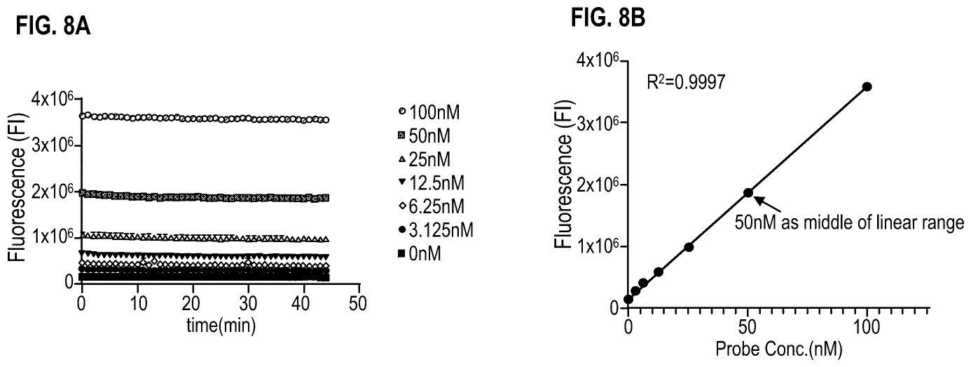

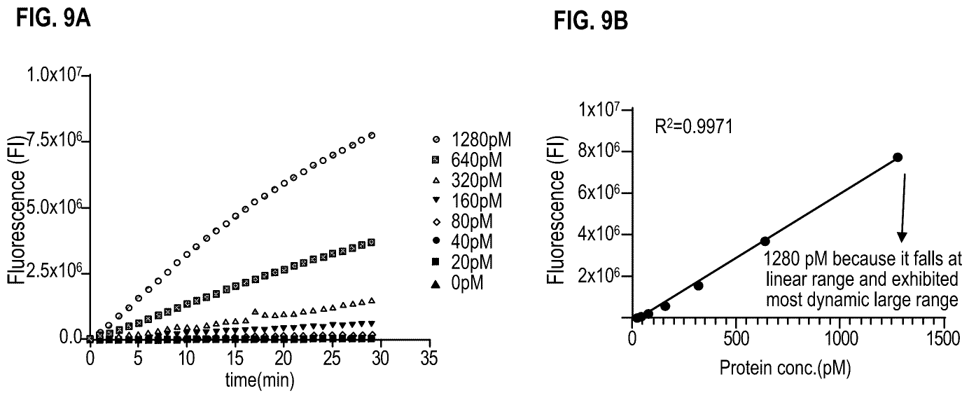

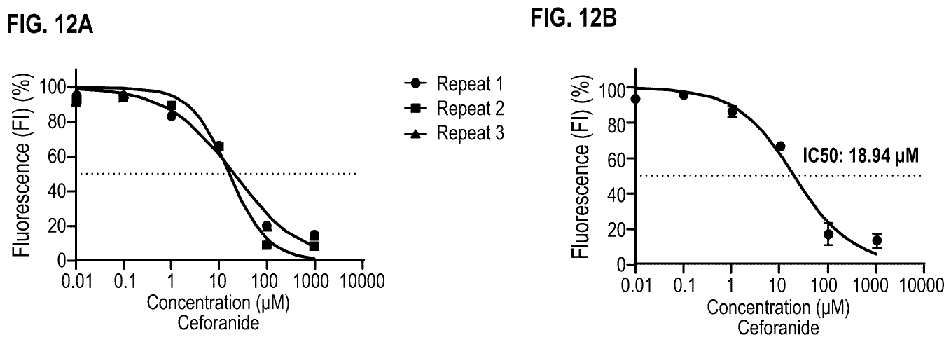

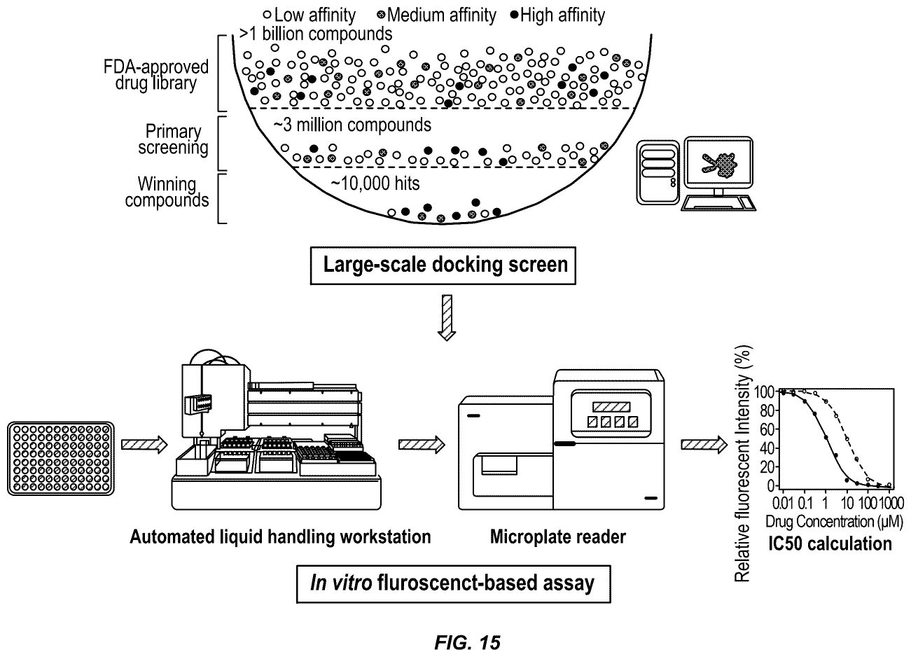

In some embodiments, the present disclosure provides an optimized in vitro assay for measuring DNASE1L3 activity and screening nuclease inhibitors, offering improved linearity, sensitivity, and scalability over conventional methods. The assay utilizes a fluorescent hydrolysis probe (e.g., 5′-FAM-labelled, 3′-quencher) in a reaction buffer (20 mM Tris-HCl, pH 7.5, 2 mM MgCl₂, 2 mM CaCl₂), 0.01% Tween-20). Recombinant DNASE1L3 is added at concentrations of 10-2000 pM (optimal: 1280 pM), and probe is used at 10-100 nM (optimal: 50 nM). Fluorescence (excitation 468 nm, emission 530 nm) is monitored over time at 37° C. This system achieves a linear relationship between fluorescence intensity and both probe and enzyme concentration (FIGS. 8A-9B), enabling accurate IC₅₀ determination (e.g., ceforanide: 18.94 μM; FIG. 12). The assay is adaptable to 96- or 384-well formats for high-throughput screening (FIG. 15).

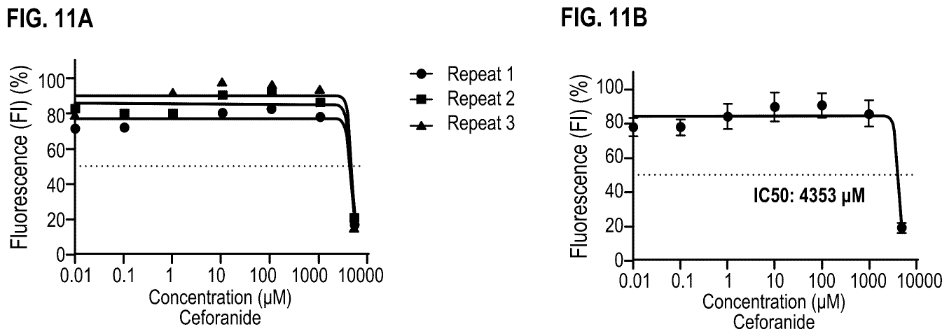

In some embodiments, the fluorescent probe is a single-stranded DNA oligonucleotide labelled with a 5′ fluorophore (e.g., FAM) and a 3′ quencher (e.g., IABkFQ), without internal quenchers such as ZEN. The probe sequence and length are optimized for efficient cleavage by DNASE1L3, providing a linear fluorescence increase over a probe concentration range of 10-100 nM (optimal: 50 nM) and enzyme concentration of 10-2000 pM (optimal: 1280 pM) (FIGS. 8A-8B, 9A-9B). Unlike prior probes incorporating internal ZEN quenchers (Barra et al., Clin. Biochem., 2015, 48(15):976-81), which increase synthesis cost and complexity, the present probe uses only terminal labels, reducing cost while maintaining high sensitivity. Furthermore, the probe is specifically calibrated for use with purified recombinant DNASE1L3, enabling reproducible IC₅₀ determination in high-throughput formats (FIGS. 11-14), which is not achievable with probes designed for total DNase activity in crude biological samples. In contrast to prior endogenous DNase assays using crude plasma or serum (Barra et al., Clin. Biochem., 2015), which measure total nuclease activity and lack linearity or specificity, the present assay uses purified recombinant DNASE1L3, eliminating interference and enabling precise, reproducible inhibitor profiling.

### A. Assessments of Activity of DNA Nucleases Using Fluorescence-Based Approach

To assess how effectively the candidate drugs identified by in-silico approaches mentioned above could reduce the activity of DNA nucleases, we developed a fluorescence-based method. For such an approach, a fluorogenic double-stranded DNA probe is designed with a 6-FAM (6-Carboxyfluorescein) fluorophore at the 5′ end and at the 3′ end with an IABGQ quencher (the first strand: 5′-6-FAM-CTCCAGCTCCACCTGAACGGCC (SEQ ID NO: 1)-IABGQ-3′; the second strand: 5′-6-FAM-GGCCGTTCAGGTGGAGCTGGAG (SEQ ID NO: 2)-IABGQ-3′). When the DNA probe is intact, the proximity of the fluorophore and quencher results in fluorescence quenching, and the probe does not emit a significant fluorescence signal, but when the nuclease enzyme (e.g. DNASE1 or DNASE1L3) cleaves the DNA probe, the fluorophore and quencher become separated and emit a fluorescence signal, which can be detected at a peak wavelength of 530 nM. Theoretically, the intensity of the fluorescence signal is proportional to the number of DNA probes that has been cleaved by the nuclease enzyme, thus reflecting the enzymatic activity of a DNA nuclease being tested.

Before the testing assay, we prepared the double-stranded fluorescent DNA probe with a concentration of 10 μM. Equal volumes of the equimolar oligonucleotides in a PCR tube were mixed, in which the 10× annealing buffer was diluted by RNase-free H₂O. The annealing step was carried out in the ProFlex™ PCR System (Life Technologies, Thermo Scientific) according to the following procedure:
1. Heat to 95° C. and maintain the temperature for 2 min.
2. Cool to 25° cover 45 min.
3. Cool to 4° C. for temporary storage.

### 1. Correlation Between Fluorescent Signals and Probe Concentrations

To test the feasibility of this method, we firstly incubated DNASE1L3 with different concentrations of fluorescence probes at the temperature of 37° C. The fluorescent DNA probe was performed to a 10-fold dilution from original 10 μM, based on this, a 2-fold serial dilution was made, thus getting a range of concentrations: 0 nM, 31.25 nM, 62.5 nM, 125 nM, 250 nM, 500 nM, 1 μM.

It is noted that DNASE1L3 and DNASE1 require divalent cations for hydrolysing double-stranded DNA, so the reactions were performed with both 2 mM Ca²⁺ and 2 mM Mg²⁺ existing. Then reaction buffer was prepared consisting of final 20 mM Tris-HCl at pH 7.5, 2 mM MgCl₂, 2 mM CaCl₂), 0.01% v/v Tween 20 detergent. After thoroughly mixing, 48 μL of reaction buffer was aliquoted, after which 6 μL probe was added into each tube at seven concentrations ranging from 0 to 100 nM, following adding 6 μL 200 pM enzyme in a final 60 μL reaction volume.

After mixing all the components well and spinning down, 50 μl aliquots were withdrawn of each reaction to the costar 96-well plate flat bottom (Corning Inc.). In the CLARIOstar^{PLUS} Microplate reader (BMG MABTECH), the excitation was set at 482 nm and the emission was measured at 530 nm while the temperature is 37° C. during time courses recording. The fluorescence value was recorded every 1 min at varying probe concentrations 0 nM, 3.125 nM, 6.25 nM, 12.5 nM, 25 nM, 50 nM, and 100 nM (FIG. 8A). For each probe concentration at 30 minutes, we generated a linearity curve showing that at this probe concentrations. There is a linear relationship between probe concentration and fluorescence without saturation (FIG. 8B). In some embodiments, an optimal probe concentration for the reaction can be, but not limited to, 10 nM, 20 nM, 30 nM, 40 nM, 50 nM, 60 nM, 70 nM, 80 nM, 90 nM, or 100 nM. In one particular embodiment, the optimal probe concentration is about 50 nM. In some embodiments, an optimal incubation time for the reaction can be anytime between 1 minute and 60 minutes, for examples, 5 minutes, 10 minutes, 20 minutes, 30 minutes, 40 minutes, 50 minutes, or 60 minutes. In one particular embodiment, the incubation time is about 30 minutes.

### 2. Correlation of Fluorescent Signals and Enzyme Concentrations

In the following experiments, the relation between fluorescence signal and concentration of the enzyme was investigated. A standard reaction was conducted in the reaction buffer [20 mM Tris-HCl (pH 7.5), 2 mM MgCl₂, 2 mM CaCl₂), 0.01% v/v Tween 20 detergent, 50 nM probe]. 6 μL of different concentrations of DNASE1L3 were added to a final volume of 60 μL, after mixing the solution rapidly, the reaction was incubated at the indicated 37° C. Fluorescence intensities were recorded every 1 min.

FIG. 9A shows a kinetics of fluorescence intensive of each reaction at different enzyme concentrations. We observed even low pM ranges of the DNASE1L3 concentrations can produce increased fluorescence emission over the time course. FIG. 9B shows a linear correlation between fluorescence signal and DNASE1L3 concentration at 30 minutes of the reaction. All tested concentrations of enzymes fell within the linear range of fluorescence intensity, and the fluorescence intensity at 1280 pM of DNASE1L3 exhibited the most dynamic signal range. As such, 1280 pM of DNASE1L3 was chosen as the optimal nuclease concentration for the reaction. In one embodiment, the enzyme concentration of 1280 pM could be used for determining IC50 (the half-maximal inhibitory concentration) of a certain inhibitor. In another embodiment, the linear correlation can be used to as calibration curve, reflecting or determining the enzyme activity in a testing sample before and after the addition of an inhibitor.

### B. Methods for Measuring DNase Activities

In one aspect, the present disclosure provides methods of assaying deoxyribonuclease (DNase) activity. The assaying can include multiple assay reactions at varying probe concentrations and/or DNase concentrations. The measured fluorescent signals over the varying concentrations can show linear behavior. Example methods are described below.

The measurement of DNase activity in the present disclosure can be based on the initial velocity of probe cleavage, which necessarily requires monitoring fluorescence intensity as a function of time in each individual reaction. To establish linear assay conditions, (a) a series of parallel reactions is performed in which either the probe concentration (typically 10-100 nM) or the DNASE1L3 concentration (typically 10-2000 pM) is systematically varied while keeping the other component constant; (b) each reaction mixture is incubated at 35-40° C. (preferably 37° C.); and (c) fluorescence intensity is recorded kinetically (e.g., every 1-2 minutes for 15-60 minutes) or at least at two time points (e.g., t=0 and t=30-45 min) during the linear phase of the reaction.

The initial rate of fluorescence increase (ΔRFU/min) in each reaction is then plotted against the varied concentration, yielding the linear ranges shown in FIG. 8B (probe) and FIG. 9B (enzyme). The concentrations providing the maximum linear slope (typically 50 nM probe and 1280 pM DNASE1L3) are selected for all subsequent inhibitor screening assays (see Section V). In inhibitor screening, the same kinetic measurement (initial rate over the first 30-45 minutes) is used to quantify the percentage of residual DNase activity in the presence of each candidate compound relative to a vehicle-only control (FIGS. 11-14).

A method for measuring DNase activities can perform a set of assays. Each assay can involve a different reaction mixture having a different probe concentrations or different DNase concentrations. Thus, the set of assays can have different probe concentrations or different DNase concentrations. Each of the set of assays can include performing the following steps.

In a 1st step, a fluorescent probe can be contacted with a DNase in a reaction mixture. Contacting may be performed by any suitable method known in the art, including but not limited to: (a) bulk solution phase in tubes, vials, or reservoirs; (b) microplate format; (c) droplet-based microfluidics, including digital droplet assays in which individual reaction droplets are generated, merged, incubated, and read (e.g., using water-in-oil droplets or nanowell-based droplet arrays); (d) microarray or solid-phase format in which the fluorescent probe is immobilised on a solid substrate (glass slide, bead, hydrogel, polymer brush, gold surface, or silicon chip) and the DNase solution is applied thereto, or vice versa; (e) paper-based or lateral-flow formats wherein the probe is dried or immobilised on cellulose or nitrocellulose and rehydrated with the DNase-containing sample; (f) single-molecule or nanowell arrays (e.g., surfaces or chips containing femtolitre wells in which one or a few DNase molecules are confined with the probe); (g) acoustic droplet ejection or other non-contact dispensing techniques; (h) electrowetting-on-dielectric (EWOD) digital microfluidics; (i) surface plasmon resonance (SPR), biolayer interferometry (BLI), or quartz crystal microbalance (QCM) sensors on which the probe is tethered and DNase binding/cleavage is monitored in real time; (j) bead-based assays (magnetic or non-magnetic beads) in suspension or in column format; and (k) any combination of the above.

In some preferred embodiments, the contacting occurs in solution phase in multi-well plates (e.g., 384-well or 1536-well) to enable high-throughput screening. In other embodiments, droplet microfluidics or solid-phase immobilization is used to reduce reagent consumption to the picolitre or femtolitre scale and/or to enable single-molecule resolution. In some embodiments, the reaction mixture comprises Tris-HCl, MgCl₂, and/or CaCl₂). As disclosed herein, the concentration of Tris-HCl in the reaction mixture may be about 10-30 mM, such as 10, 12, 14, 16, 18, 20, 22, 24, 26, 28, or 30 mM, or any intermediate value within this range. In particular embodiments, the concentration of Tris-HCl in the reaction mixture is about 20 mM. As disclosed herein, the concentration of MgCl₂ in the reaction mixture may be about 1-3 mM, such as 1, 1.2, 1.4, 1.6, 1.8, 2, 2.2, 2.4, 2.6, 2.8, or 3 mM, or any intermediate value within this range. In particular embodiments, the concentration of MgCl₂ in the reaction mixture is about 2 mM. As disclosed herein, the concentration of CaCl₂) in the reaction mixture may be about 1-3 mM, such as 1, 1.2, 1.4, 1.6, 1.8, 2, 2.2, 2.4, 2.6, 2.8, or 3 mM, or any intermediate value within this range. In particular embodiments, the concentration of CaCl₂) in the reaction mixture is about 2 mM. In some embodiments, the pH value of the reaction mixture is about between 6 and 9, preferably about 7.5.

In some embodiments, the concentration of the fluorescent probe in the reaction mixture is about 10-100 nM, such as 10, 20, 30, 40, 50, 60, 70, 80, 90, or 100 nM, or any intermediate value within this range. In particular embodiments, the concentration of the fluorescent probe in the reaction mixture is about 50 nM. As disclosed herein, the fluorescent probe may comprise a single-stranded or double-stranded DNA oligonucleotide linked with a fluorophore (e.g., FAM) and a quencher (e.g., IABkFQ) at each end. In some embodiments, the fluorescent probe is a single-stranded DNA oligonucleotide labeled with a 5′ fluorophore (e.g., FAM) and a 3′ quencher (e.g., IABkFQ), without internal quenchers such as ZEN. The probe sequence and length are optimized for efficient cleavage by DNASE1L3.

In some embodiments, the concentration of the DNase in the reaction mixture is about 10-2000 pM, such as 10, 20, 30, 40, 50, 60, 70, 80, 90, 100, 200, 500, 1000, 1500, or 2000 nM, or any intermediate value within this range. In some embodiments, the DNase can be a recombinant human DNASE1L3 described herein.

In a 2nd step, the reaction mixture can be incubated for a period of time (e.g., 1-60 min, such as 1, 10, 15, 20, 30, 40, 50, or 60 minutes, or any intermediate value within this range). In some embodiments, the incubation temperature is from 35° C. to 40° C., such as about 35, 36, 37, 38, 39, or 40° C., preferably at about 37° C.

In a 3rd step, fluorescence intensity can be measured. When the set of measurements of the fluorescence intensity values for the different concentrations of the different assays are plotted, the fluorescence response can be linear with the changes in concentration, e.g., as shown in FIGS. 8B and 9B.

The set (series) of assays can be performed separately (e.g., separate reaction vessels) or using a same reaction vessel where more probe or DNase is added to obtain an increasing concentration, corresponding to a new assay measurement.

In some embodiments, the fluorescence intensity is measured after or during the incubation step by exciting the fluorophore (e.g., FAM or equivalent) at a wavelength in the range of 450-500 nm, and detecting the emitted fluorescence at a wavelength in the range of 510-560 nm (preferably 518-545 nm, most preferably 525-540 nm or approximately 530 nm) . . . . The excitation wavelength range is preferably 460-485 nm, most preferably 465-475 nm or approximately 468 nm or 470 nm) and the emission wavelength range is preferably 518-545 nm, most preferably 525-540 nm or approximately 530 nm or 535 nm).

These wavelength ranges are compatible with: a) 6-carboxyfluorescein (FAM) and closely related fluorescein derivatives (e.g., 5-FAM, 6-FAM, JOE, TET, HEX, ROX, Cy3, ATTO488, Alexa Fluor 488); b) standard plate-reader filter sets and monochromators found on essentially all commercial microplate readers (Tecan Spark/SparkControl, PerkinElmer EnVision/EnSight, BioTek Synergy/Cytation, Molecular Devices SpectraMax iD5/13x, BMG LABTECH PHERAstar, etc.); and c) most fluorescence microscopes, flow cytometers, droplet readers, and qPCR instruments.

In the specific examples disclosed herein (FIGS. 8-14), fluorescence was measured using excitation at **468±10 nm** and emission at **530±15 nm** on a Tecan Spark multimode reader, with readings taken every 1-2 minutes during kinetic assays or as a single end-point measurement. In other embodiments (e.g., droplet or nanowell formats), excitation may be performed with 488 nm or 473 nm lasers and emission collected with 510-540 nm bandpass filters.

Accordingly, to ensure assay linearity, probe concentrations are tested over a range of 10-100 nM (FIG. 8A), and DNASE1L3 concentrations are tested over a range of 10-2000 pM (FIG. 9A). In a subsequent step, fluorescence intensity can be monitored over a period of time (e.g. 1-60 minutes) at 37° C. to capture the time-dependent increase in signal during probe cleavage. The optimal working concentrations, at 50 nM probe and 1280 pM DNASE1L3, are selected where fluorescence increases linearly with both probe concentration (FIG. 8B) and enzyme concentration (FIG. 9B), without saturation. This calibration step to identify optimal working concentrations may performed once to establish assay conditions. Subsequent inhibitor screening can use fixed optimal concentrations (50 nM probe, 1280 pM DNASE1L3) with varying inhibitor doses to generate dose-response curves and IC₅₀ values (FIGS. 11-14). Unlike prior probes incorporating internal ZEN quenchers (Barra et al., Clin. Biochem., 2015), which increases cost and complexity, the present disclosure provides a probe which can use only terminal labels (5′ fluorophore and 3′ quencher), and achieves comparable linearity and sensitivity for high-throughput screening. Furthermore, linearity is established using purified recombinant DNASE1L3, enabling reproducible, high-throughput IC₅₀ determination, features not disclosed in assays measuring total DNase activity in crude plasma or serum. The probe described herein utilizers only terminal labels (e.g., 5′ fluorophore and 3′ quencher) without any internal quenchers. This design enables cost-effective synthesis while maintaining linearity in the purified DNASE1L3 system.

In a particular embodiment, the present disclosure provides a method of calibrating DNASE1L3 activity, that can include: (a) incubating DNASE1L3 with a probe over a range of probe concentrations from 10 nM to 100 nM, or over a range of DNASE1L3 concentrations from 10 pM to 2000 pM; (b) measuring fluorescence intensity over time at a temperature between 35° C.-40° C., preferably 37° C.; and (c) selecting a working probe concentration or a working DNASE1L3 concentration where fluorescence increase is linear with respect to the range of probe concentrations or the range of DNASE1L3 concentrations, thereby calibrating DNASE1L3 activity.

### IV. Example Inhibition Tests of Various Drugs

This section provides exemplary methods to test various nuclease inhibitors.

A. Inhibition test of Ceforanide for DNASE1L3 and DNASE1

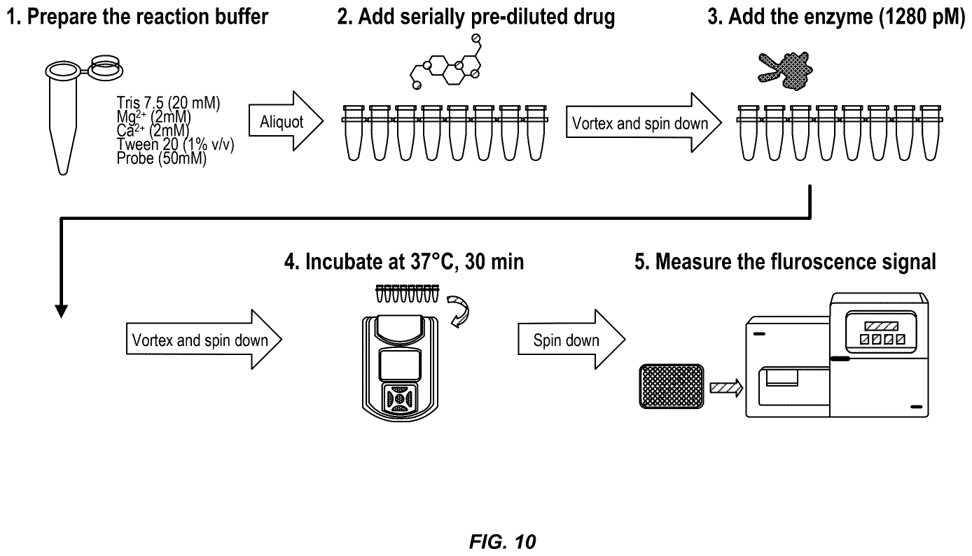

FIG. 10 shows the schematic of the experimental workflow for testing the inhibition of DNASE1 or DNASE1L3 by a drug compound using a fluorescent probe as the substrate. The workflow includes 5 steps: 1, the reaction buffer was freshly prepared and aliquoted to 8-Tube Strip, which consists of 20 mM Tris-HCl, pH 7.5, 2 mM MgCl₂, 2 mM CaCl₂), 0.01% v/v Tween 20, 50 nM probe. 2, Ceforanide was serially diluted in DMSO to create a range of concentrations. 3, 1280 pM of the enzyme was added to the reaction mixture. 4, after thoroughly vortexing and spinning down, the mixture was incubated at 37° C. for 30 minutes. 5, an equal volume of reaction buffer (50 μL) was transferred to the costar 96-well plate flat bottom. The emission was measured at a peak wavelength of 530 nm, while the excitation was set at 482 nm. Ultimately, the drug's inhibitory impact was measured based on the fluorescence signal altered. In one embodiment, a reaction buffer containing the following components was prepared: 20 mM Tris-HCl at pH 7.5, 2 mM MgCl₂, 2 mM CaCl₂), 0.01% v/v Tween 20 detergent, and 50 nM fluorescent DNA probe. Mixed well for following enzyme activity and detection. The 50 mM Ceforanide (MedChemExpresss) solution was used as stock in DMSO, which was serially diluted to create a range of concentrations varying from 100 nM to 10 mM, allowing the testing of inhibitory effect of the drug at different concentrations.

Then equal volume of reaction buffer (48 μL) was added to 8-Tube Strip, 6 μL Ceforanide (diluted from DMSO stock) were added into each tube at final concentrations ranging from 10 nM to 1 mM or 5 mM in ten-fold dilutions (final DMSO concentration 10%). 6 μL enzyme (DNASE1 or DNASE1L3) was added to each reaction mixture subsequently, reaching a final enzyme concentration of 1280 pM with a net volume of 60 μL. The samples were thoroughly mixed externally using a vortex mixer for approximately 30 s and centrifuge at 2000 rpm for 1 min.

After mixing the drug and the reaction buffer well, the fixed concentration of the enzyme (1280 pM) is added to the reaction mixture. The above steps are operated at 4° C. to avoid the reactions proceeding before the desired incubation time. Following thoroughly vertexing and spinning down, the mixture is subjected to incubated at the optimal temperature (37° C.) for 30 minutes. After brief centrifugation to draw all moisture away from the lid, equal volume of reaction buffer (50 μL) would be transferred to the costar 96-well plate flat bottom. In the plate reader, the excitation was set at 482 nm and the emission was measured at a peak wavelength at 530 nm. Next, the inhibitory effect of the drug would be quantified according to fluorescence signal changed which is likely related to the activity of the enzyme.

In the subsequent experiments, we investigated whether the Ceforanide could exhibit inhibitory effect on DNASE1 or DNASE1L3 following the protocol depicted above. An array of Ceforanide concentrations were incubated with DNASE1 (Thermo, Catalog number: 18068015) when 50 nM probe was used as substrate in the reaction. The anti-nuclease efficacy of the compound was monitored by recording the fluorescent emission over 30 minutes of incubation at 37° C. This fluorescence signal is likely related to the activity of the enzyme and the normalized fluorescence intensity can be used to quantify the inhibitory effect of the drug. The normalized intensity is calculated as (F−F_{base})/F₀, where F is the fluorescence intensity for a given concentration, F_{base} is the baseline intensity of the probe, and F₀ is the fluorescence intensity when drugs are not added. All fluorescence measurements were conducted in a CLARIOstar^{PLUS} Microplate reader (BMG MABTECH). The data was plotted in the GraphPad Prism and IC50 was calculated according to “log (inhibitor) vs. response—Variable slope (four parameters)”. Based on three independent experiments, it was demonstrated that the IC50 of Ceforanide against DNASE1 was 4353 μM (FIG. 11).

Also, the relationship between Ceforanide concentration and the activity of DNASE1L3 was conducted under a desired condition. As FIGS. 12A and 12B shown, a prominent decrease in fluorescent signal was observed with increased Ceforanide concentration, indicating the enzyme's double-strand cleavage was inhibited. Fitting the data according to triplicates testing revealed IC50 of Ceforanide against DNASE1L3 was determined as 18.94 μM, exerting a stronger inhibitory effect than on DNASE1. The results underscored that Ceforanide, may find its role in inhibition of DNASE1L3 .

### B. Inhibition Test of Cefuroxime Sodium on DNASE1L3

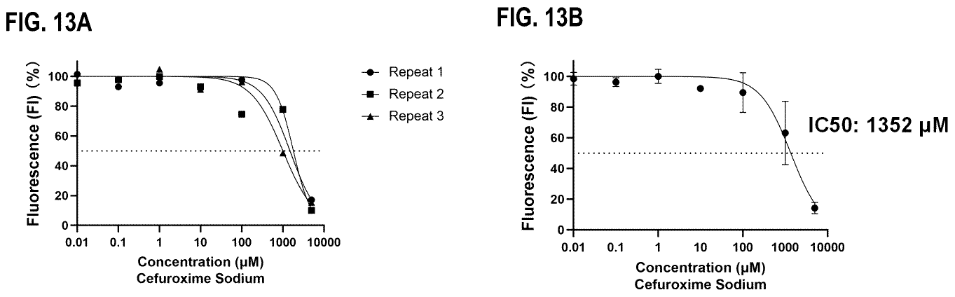

We further examined cefuroxime sodium's activity to inhibit DNASE1L3. As shown in FIGS. 13A and 13B, a prominent decrease in fluorescent signal was observed with increased concentrations of cefuroxime sodium, indicating the DNASE1L3 's cleavage function was inhibited by cefuroxime sodium. Fitting the data according to triplicates testing revealed IC50 of cefuroxime sodium against DNASE1L3 was determined as 1352 μM.

### C. Inhibition Test of Minocycline on DNASE1L3

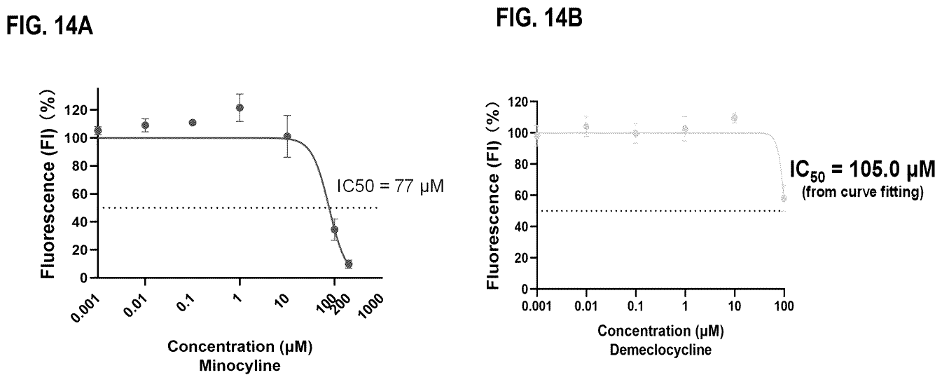

We further examined minocycline's inhibition ability on human DNASE1L3. As shown in FIG. 14A, a prominent decrease in fluorescent signal was observed with increased concentrations of minocycline, indicating the DNASE1L3 's cleavage function was inhibited by minocycline. Fitting the data according to triplicates testing revealed IC50 of minocycline against DNASE1L3 was determined as 77 μM.

### D. Inhibition Test of Demeclocycline on DNASE1L3

We further examined demeclocycline's inhibition ability on human DNASE1L3. As shown in FIG. 14B, a prominent decrease in fluorescent signal was observed with increased concentrations of demeclocycline, indicating the DNASE1L3 's cleavage function was inhibited by demeclocycline. Fitting the data according to triplicates testing revealed IC50 of demeclocycline against DNASE1L3 was determined as 105.0 μM.

### E. Inhibition Test of Rifampin on DNASE1L3

Rifampin's inhibition ability on human DNASE1L3 was also tested. We also observed a decrease in fluorescent signal with increased concentrations of rifampin, indicating rifampin can inhibit the DNASE1L3 's cleavage function.

### V. Platform for Large-Scale Screening of Drugs that Inhibit DNA Nucleases Using Fluorescence-Based Approach

The present disclosure further provides methodologies for screening candidate compositions (e.g., drugs) that inhibit DNA nucleases. Non-limiting examples of methods include computational simulations, fluorescence-labeled probe-based enzyme activity screening, human/mouse plasma/serum chromatin digestion assay, in vivo mouse inhibition efficiency test (e.g. degree of cfDNA level elevation), and any combination thereof. This section illustrates an exemplary platform for large-scale screening of drugs that inhibit DNA nucleases using fluorescence-based approach.

### A. Example Protocols of Large-Scale DNase Inhibitor Screenings

Here we report the possibility of using FDA-approved Ceforanide with potent anti-activity against DNASE1 and DNASE1L3 through pocket binding free energy calculation. We also refined a pipeline transferring the activity of nucleases to fluorescent signal and we implemented this strategy on drug inhibitory testing. Based on the fluorescence assay, we determined the half-maximal inhibitory concentration (IC50) values for Ceforanide against DNASE1 and DNASE1L3. Ceforanide showed IC50 values of 4353 μM and 18.94 μM against DNASE1 and DNASE1L3, respectively, indicating it is a more potent inhibitor of DNASE1L3, which is possible to reduce the clearance of cell-free DNA transiently to improve the sensitivity and the robustness of ctDNA clinical tests.

Given the high screen efficiency and easy operation steps of this approach (FIG. 15), in one embodiment, this fluorescence-based assay can be applied in large-scale screening hits against DNASE1 and DNASE1L3. As the workflow below, we raise a pipeline that with the further-optimized enzyme's pocket and calculation parameters for docking, the top-ranked molecules can be filtered after the large-scale screening the FDA-approved drugs library. The selected compounds would be subjected to test in in vitro fluorescence-based experiment, and the screening time would be sharply shortened and screening capability would be enlarged with automated liquid handling workstation. Subsequent evaluation on inhibitory activity of these drugs would be analyzed according to the fluorescent intensity value. Some embodiments can directly rely on the large-scale screening of inhibitors on the basis of fluorescence-based assay without computational simulation (e.g. bocking).

### B. Methods for Screening for Nuclease Inhibitors

In another aspect, the present disclosure provides methods of screening for nuclease inhibitors. Example methods are described below.

In a 1st step, a candidate compound can be contacted with a DNase and a fluorescent probe in a reaction mixture. In some embodiments, the reaction mixture comprises Tris-HCl, MgCl₂, and/or CaCl₂). As disclosed herein, the concentration of Tris-HCl in the reaction mixture may be about 10-30 mM, such as 10, 12, 14, 16, 18, 20, 22, 24, 26, 28, or 30 mM, or any intermediate value within this range. In particular embodiments, the concentration of Tris-HCl in the reaction mixture is about 20 mM. As disclosed herein, the concentration of MgCl₂ in the reaction mixture may be about 1-3 mM, such as 1, 1.2, 1.4, 1.6, 1.8, 2, 2.2, 2.4, 2.6, 2.8, or 3 mM, or any intermediate value within this range. In particular embodiments, the concentration of MgCl₂ in the reaction mixture is about 2 mM. As disclosed herein, the concentration of CaCl₂) in the reaction mixture may be about 1-3 mM, such as 1, 1.2, 1.4, 1.6, 1.8, 2, 2.2, 2.4, 2.6, 2.8, or 3 mM, or any intermediate value within this range. In particular embodiments, the concentration of CaCl₂) in the reaction mixture is about 2 mM. In some embodiments, the pH value of the reaction mixture is about between 6 and 9, preferably about 7.5. In some embodiments, the incubation temperature is from 35° C. to 40° C., such as about 35, 36, 37, 38, 39, or 40° C., preferably at about 37° C. For example, as used in the examples of FIGS. 8-14, the reaction mixture is incubated at about 37° C., which provides robust DNase activity across the full range without denaturation. This is consistent with standard in vitro nuclease assays (e.g., Barra et al., Clin. Biochem. 48:976-981, 2015; and Napirei et al., Biochem. J. 389:355-365, 2005).

In an optional 2nd step, DNase activity can be calibrated to ensure a linear fluorescence response with respect to both probe concentration and enzyme concentration, using the fluorescent probe method described in Section III.B. In other embodiments, the calibration could have been done before and by a different party, e.g., so the calibrated values can be used. This calibration can be achieved by performing two independent sets of parallel reactions: (i) a first set in which the probe concentration is systematically varied (e.g., 10, 20, 30, 40, 50, 75, 100 nM) while the DNASE1L3 concentration is held constant (typically at the final chosen level, e.g., 1280 pM); and (ii) a second set in which the DNASE1L3 concentration is systematically varied (e.g., 10, 50, 100, 250, 500, 1000, 1280, 2000 pM) while the probe concentration is held constant (typically at the final chosen level, e.g., 50 nM).

In each individual reaction of both sets, the procedures can comprise: (a) contacting the fluorescent probe (terminally labelled, lacking an internal quencher) with recombinant human DNASE1L3 in a reaction mixture; (b) incubating the reaction mixture at a temperature from 35° C. to 40° C. (preferably 37° C.) for a period sufficient to remain within the linear phase of probe cleavage (typically 15-60 minutes); and (c) measuring fluorescence intensity as a function of incubation time, wherein the measurement is performed either (i) kinetically by recording fluorescence at multiple time points (e.g., every 1-2 minutes; as shown in the time-course traces of FIGS. 8A and 9A), or (ii) as a single end-point reading at a pre-determined fixed time point within the linear phase (e.g., 30-45 minutes). For each reaction, the initial rate of fluorescence increase (ΔRFU/min, calculated from the linear portion of the kinetic trace or from the single end-point value normalised to incubation time) is determined. These initial rates can be plotted against the varied concentration, yielding the linear relationships shown in FIG. 8B (probe concentration) and FIG. 9B (enzyme concentration). The concentrations that produce the steepest linear slope without saturation, typically 50 nM probe and 1280 pM DNASE1L3, are selected and thereafter fixed for all subsequent high-throughput inhibitor screening assays (FIGS. 11-14).

This rate-based calibration distinguishes the present method from prior endogenous DNase assays that rely on arbitrary single end-point readings in complex biological matrices without prior linearity validation (see, e.g., Barra et al., Clin. Biochem. 48:976-981, 2015).

In a 3^{rd} step, the candidate compound is identified as a nuclease inhibitor if the DNase activity in the presence of the candidate compound is reduced by a specified threshold relative to a control condition performed in parallel under identical conditions. The control condition (also referred to as “no-inhibitor control”, “vehicle control”, or “negative control”) consists of the complete reaction mixture containing: (a) recombinant DNase (e.g., DNASE1L3 at the calibrated concentration, typically 1280 pM), (b) fluorescent probe (typically 50 nM), (c) reaction buffer (e.g., 20 mM Tris-HCl pH 7.5, 2 mM MgCl₂, 2 mM CaCl₂)), and (d) the identical volume of solvent/vehicle used to dissolve the candidate compound (e.g., DMSO, water, or buffer) but lacking the candidate compound itself. No placebo or irrelevant molecule is required; the control simply omits the candidate compound while including the vehicle at the same final concentration (typically ≤1-2% v/v DMSO). This vehicle-only control represents 100% DNase activity. The specified threshold for identifying an inhibitor can be a reduction of DNase activity to ≤70% (i.e., ≥30% inhibition), ≤60% (≥40% inhibition), ≤50% (≥50% inhibition), ≤40% (≥60% inhibition), or ≤30% (≥70% inhibition) of the vehicle-only control. In particular embodiments, a candidate is identified as an inhibitor when residual activity is ≤50% of the vehicle-only control (IC₅₀ determination shown in FIGS. 11-14).

An optional 4th step can include: calculating an IC₅₀ value for the candidate compound. As disclosed herein, the DNase can be a recombinant human DNASE1L3. In some embodiments, the method is performed in a high-throughput format using a multi-well plate.

In a particular embodiment, the present disclosure provides a method of screening a nuclease inhibitor using a calibrated DNASE1L3 including: (a) contacting a candidate with the calibrated DNASE1L3 and the probe at the selected working concentrations; (b) measuring residual DNASE1L3 activity; and (c) identifying the candidate as a nuclease inhibitor if the residual DNASE1L3 activity is reduced by at least 50% relative to a no-candidate control.

### VI. Nuclease Inhibitors

As disclosed herein, any nuclease inhibitor that can prevent or limit the activity of a nuclease (such as a deoxyribonuclease) in a subject can be used to increase cfDNA level in a bodily fluid of the subject. The term “nuclease inhibitor” refers to any molecular, compound, or agent that can control or modify the activity of a nuclease. In some instances, the nuclease inhibitor comprises an antibiotic. In other instances, the nuclease inhibitor comprises a non-antibiotic molecule such as a nucleotide, protein, or synthetic organic and inorganic compound. In some embodiments, the nuclease inhibitor is an FDA-approved drug.

### A. Antibiotics

In some embodiments, the nuclease inhibitor is an antibiotic drug. The term “antibiotic” or “antibiotic drug” refers to an antimicrobial molecule active against bacteria. Antibiotics are commonly classified based on their mechanism of action, chemical structure, or spectrum of activity. Most antibiotics target bacterial functions or growth processes, thereby having bactericidal activities and killing the bacteria. For examples, penicillins and cephalosporins target bacterial cell wall; polymyxinsor targets bacterial cell membrane, and rifamycins, lipiarmycins, quinolones, and sulfonamides interfere with essential bacterial enzymes. Some antibiotics, such as macrolides, lincosamides, and tetracyclines, are protein synthesis inhibitors to inhibit bacterial further growth. The antibiotics can be either “narrow spectrum” antibiotics targeting specific types of bacteria, such as gram-negative or gram-positive, or “broad-spectrum” antibiotics affect a wide range of bacteria.

The present disclosure provides a surprising discovery that an antibiotic can be used as a nuclease inhibitor to increase cell-free DNA (cfDNA) level in a bodily fluid of a subject. Non-limiting examples of the antibiotic drug include cephalosporin, sulfonamide, tetracycline, rifamycin, polyene macrolides, or any derivative, fragment, or combination thereof. In some embodiments, the nuclease inhibitor is an antibiotic cephalosporin. Non-limiting examples of cephalosporin include ceforanide, cefuroxime sodium, cefamandole sodium, cefaclor, cefdinir, or any derivative, fragment, or combination thereof. In a particular embodiment, the nuclease inhibitor is antibiotic ceforanide. In some embodiments, the nuclease inhibitor is an antibiotic sulfonamide. Non-limiting examples of sulfonamide include sulfadiazine, sulphapyridine, or any derivative, fragment, or combination thereof. In some embodiments, the nuclease inhibitor is an antibiotic tetracycline. Non-limiting examples of tetracycline include minocycline, doxycycline, demeclocycline, tetracycline, omadacycline, sarecycline, or any derivative, fragment, or combination thereof. In some embodiments, the nuclease inhibitor is an antibiotic rifamycin. Non-limiting examples of rifamycin include rifaximin, rifabutin, rifamycin, rifampin (rifampicin), rifapentine, rifalazil, or any derivative, fragment, or combination thereof. In some embodiments, the nuclease inhibitor is an antibiotic polyene macrolide. Non-limiting examples of polyene macrolide include amphotericin B, nystatin, natamycin, or any derivative, fragment, or combination thereof. In some embodiments, the antibiotic drug is an FDA-approved drug.

Without being bound by theory or intending to limit the scope of the invention, the nuclease inhibitor can be an antibiotic selected from the group listed in Table 3.

TABLE 3
Antibiotics as nuclease inhibitors identified
in the present disclosure.

Amoxicillin
Ampicillin
Azithromycin
Besifloxacin
Capreomycin
Cefaclor
Cefamandole
Cefdinir
Cefonicid
Cefotetan
Cefotiam hydrochloride
Cefprozil
Ceftazidime
Ceftriaxone
Cefuroxime
Cephalexin
Clindamycin
Demeclocycline
Doxycycline
Eravacycline
Ertapenem
Erythromycin
Fidaxomicin
Linezolid
Methacycline
Minocycline
Moxifloxacin
Nafcillin
Natamycin
Neomycin
Omadacycline
Paromomycin
Penicillin G
Penicillin V
Quinupristin
Rifabutin
Rifampin
Rifamycin
Rifapentine
Rifaximin
Sarecycline
Streptomycin
Telithromycin
Tetracycline
Tigecycline
Tobramycin
Vancomycin

### 1. Cephalosporins

As disclosed herein, an antibiotic cephalosporin can be used as a nuclease inhibitor to enhance cfDNA levels in a bodily fluid of a subject. In some embodiments, the antibiotic cephalosporin is one of the first generation cephalosporins selected from cephalothin, cephaloridine, cephapirin, cefazolin, cephalexin, cephradine, and cefadroxil. In some embodiments, the antibiotic cephalosporin is one of the second generation cephalosporins selected from cepfamandole, cefoxitin, cefotiam, cefaclor, cefuroxime, cefotetan, ceforanide, cefonicid, cefprozil, cefoxitin, cefotetan, and cefmetazole. In some embodiments, the antibiotic cephalosporin is one of the third generation cephalosporins selected from ceftiofur, ceftriaxone, cefsulodin, cefotaxime, cefoperazone, ceforanide, ceftazidime, cefpodoxime, cefixime, ceftibuten, cefdinir, ceftizoxime. In some embodiments, the antibiotic cephalosporin is a fourth-generation cephalosporin such as cefepime. In some embodiments, the antibiotic cephalosporin comprising free base acid stable forms can be administered orally. Examples of oral preparations are cephalexin, cephradine, cefadroxil and cefachlor. In some embodiments, the antibiotic cephalosporin comprising sodium salt derivatives can be administered parenterally, such as subcutaneous, intramuscular, intravenous, or intrathecal administration. In some embodiments, the antibiotic cephalosporin is selected from the group consisting of ceforanide, cefuroxime sodium, cefamandole sodium, cefaclor, cefdinir, or a derivative thereof.

### a) Ceforanides

In a particular embodiment, the nuclease inhibitor is antibiotic ceforanide. Ceforanide is an FDA-approved antibacterial drug under the name Precef that has been used to treat a variety of infections, including skin and soft tissue infections, pulmonary infections, urinary tract infections, bone and joint infections, and endocarditis. In some embodiments, ceforanide comprises a structure shown below:

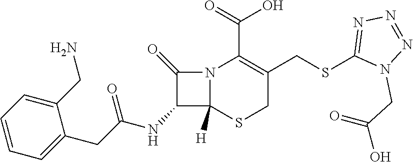 [SMILES: NCc1ccccc1CC(=O)N[C@@H]1C(=O)N2C(C(=O)O)=C(CSc3nnnn3CC(=O)O)CS[C@H]12]

Ceforanide (PubChem CID 43507) is a semisynthetic second-generation cephalosporin antibiotic that has a longer elimination half-life than other cephalosporins. Ceforanide has been approved to be effective against various coliforms, such as Escherichia coli, Klebsiella, Enterobacter, and Proteus, and against various strains of Salmonella, Shigella, Hemophilus, Citrobacter, and Arizona species. Ceforanide causes inhibition of bacterial cell wall synthesis by inactivating penicillin binding proteins (PBPs) thereby interfering with the final transpeptidation step required for cross-linking of peptidoglycan units which are a component of the cell wall. This results in a reduction of cell wall stability and causes cell lysis.

The present disclosure provides an unexpected discovery that cefornaide can bind to a catalytic pocket of DNASE1 and DNASE1L3 (FIGS. 4 and 5) and significantly inhibit the two nucleases' activities in vitro (FIGS. 11 and 12). As disclosed herein, the nuclease inhibitor, such as cefornaide, binds to one or more catalytic pockets of a nuclease. In some embodiments, the nuclease inhibitor binds on or near (e.g., +3 residues of) the ARG29 residue of DNASE1L3. In some embodiments, the nuclease inhibitor binds on or near (e.g., +3 residues of) the TYR211 residue of DNASE1.

In some embodiments, the nuclease inhibitor, such as cefornaide, is administered to a subject with a dosage of about 1-50 uM per kilogram body weight. In some embodiments, the therapeutically effective amount of cefornaide to be administered is about 10-30 uM per kilogram body weight. In some embodiments, the therapeutically effective amount of cefornaide is about 20 uM per kilogram body weight.

### b) Cefuroximes

In a particular embodiment, the nuclease inhibitor is antibiotic cefuroxime. Cefuroxime, an FDA-approved antibacterial drug under the name Zinacef among others, is a second-generation cephalosporin antibiotic used to treat and prevent a number of bacterial infections such as pneumonia, meningitis, otitis media, sepsis, urinary tract infections, and Lyme disease. Cefuroxime can be administered orally, intravenously, or intramuscularly. In some embodiments, the cefuroxime comprises a structure shown below:

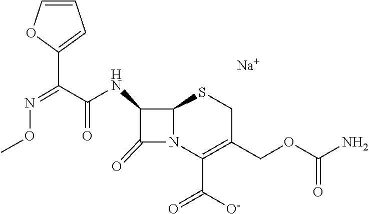 [SMILES: CO/N=C(\C(=O)N[C@@H]1C(=O)N2C(C(=O)[O-])=C(COC(N)=O)CS[C@H]12)c1ccco1 ; [Na+]]

In some embodiments, the cefuroxime is cefuroxime axetil. Cefuroxime axetil is an

FDA-approved antibacterial drug under the brand name Ceftin among others, is a second-generation oral cephalosporin antibiotic. Cefuroxime axetil comprises a structure shown below:

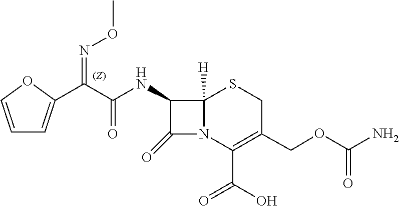 [SMILES: CO/N=C(\C(=O)N[C@@H]1C(=O)N2C(C(=O)O)=C(COC(N)=O)CS[C@H]12)c1ccco1]

### 2. Tetracyclines

As disclosed herein, an antibiotic tetracycline can be also used as a nuclease inhibitor to enhance cfDNA levels in a bodily fluid of a subject. Tetracyclines are a group of broad-spectrum antibiotic compounds and are either isolated directly from several species of Streptomyces bacteria or produced semi-synthetically from those isolated compounds. Nonlimiting examples of tetracyclines include minocycline, tetracycline, chlortetracycline, oxytetracycline, demeclocycline, lymecycline, meclocycline, methacycline, rolitetracycline, doxycycline, tigecycline, eravacycline, sarecycline, omadacycline, and any derivative thereof. In a particular embodiment, the nuclease inhibitor is minocycline.

### a) Minocycline

Minocycline, an FDA-approved antibacterial drug under the brand name Minocin among others, is a second-generation tetracycline antibiotic that is active against gram-negative and gram-positive bacteria. Minocycline can be administered orally or applied to skin. In some embodiments, a minocycline comprises a structure shown below:

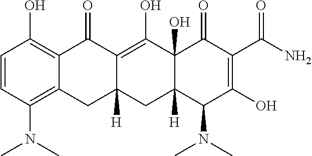 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

As disclosed herein, minocycline can be administered orally, intravenously, or applied to skin of a subject. An oral administration of minocycline in a pellet filled capsule has a half live of minocycline about 11-22 hours. An intravenous injection of minocycline has a half live of minocycline about 15-23 hours. Intravenous minocycline has a clearance of 3.36-5.7 L/h, while oral minocycline has a clearance of 3.42-4.4 L/h. The oral LD50 (lethal dose 50) in mice is 3600 mg/kg. The intraperitoneal LD50 in mice is 299 mg/kg. The subcutaneous LD50 in mice is 2290 mg/kg.

### b) Demeclocycline

Demeclocycline, an FDA-approved antibacterial drug under the brand name Declomycin, is a tetracycline antibiotic that is active against gram-negative and gram-positive bacteria, as well as certain other organisms like Mycoplasma and Chlamydia. Demeclocycline is primarily administered orally and is also used to treat syndrome of inappropriate antidiuretic hormone (SIADH) secretion. In some embodiments, a demeclocycline comprises a structure shown below:

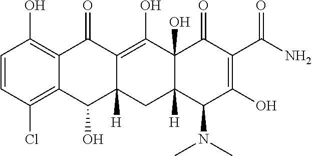 [SMILES: CN(C)[C@@H]1C(O)=C(C(N)=O)C(=O)[C@@]2(O)C(O)=C3C(=O)c4c(O)ccc(Cl)c4[C@@H](O)[C@H]3C[C@@H]12]

In some embodiments, Demeclocycline can be administered orally in tablet form (e.g., 150 mg or 300 mg doses). An oral administration of demeclocycline has a half-life of about 10-17 hours in adults with normal renal function, which may extend to 40-60 hours in patients with severe renal impairment. Intravenous administration is not commonly used for demeclocycline, and topical applications are rare but may be considered for specific formulations. Oral demeclocycline has a clearance of approximately 1.5-3.0 L/h in healthy adults, primarily via renal excretion (50-60% unchanged in urine). The oral LD50 (lethal dose 50) in mice is approximately 2,372 mg/kg. The intraperitoneal LD50 in mice is approximately 150-300 mg/kg.

### 3. Rifamycins

As disclosed herein, an antibiotic rifampin can be also used as a nuclease inhibitor to enhance cfDNA levels in a bodily fluid of a subject. The rifamycins are a broad-spectrum antimicrobial, to treat tuberculosis and works by inhibiting the microbial DNA-dependent RNA polymerase (RNAP). The rifamycins are synthesized either naturally by the bacterium Amycolatopsis rifamycinica or artificially. The rifamycin group includes the classic rifamycin drugs and the rifamycin derivatives including, but not limited to, rifampicin (or rifampin), rifabutin, rifapentine, rifalazil, rifaximin, and any derivative thereof. In a particular embodiment, the nuclease inhibitor is the classic rifamycin drug. The classic rifamycin is an FDA-approved antibacterial drug under the brand name Aemcolo. In some embodiments, the rifamycin comprises a structure shown below:

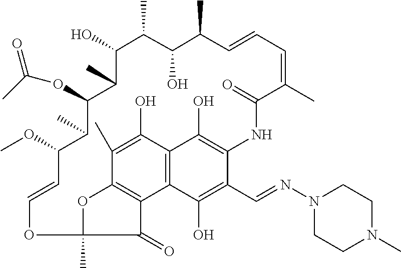

4. Polyene macrolides

As disclosed herein, an antibiotic polyene macrolide can be also used as a nuclease inhibitor to enhance cfDNA levels in a bodily fluid of a subject. Polyene macrolides (polyene antibiotics, or polyene antimycotics) are a class of antimicrobial polyene compounds that also target fungi. The polyene antimycotics are typically obtained from certain species of Streptomyces bacteria. Nonlimiting examples of polyene macrolides include amphotericin B, nystatin, natamycin (pimaricin), and any derivative thereof.

### B. Other Nuclease Inhibitors

Besides antibiotics and anti-fungal drugs, other nuclease inhibitors can be also used to increase cfDNA level in a bodily fluid of a subject. In some embodiments, the nuclease inhibitor is synephrine. Synephrine (or p-synephrine) is an alkaloid that occurs naturally in some plants and animals, and is an FDA proved drug for weight-loss, generating the adrenergic effects. In some embodiments, the nuclease inhibitor is isoprenaline. Isoprenaline (or isoproterenol) is a pure β-agonist that stimulates both β1- and β2-adrenergic receptors. Isoprenaline is an FDA proved drug used to treat a variety of conditions, including Bradycardia, Heart block, Bronchospasm, Cardiac arrest, Hypovolemic shock, Septic shock, Congestive heart failure, and Cardiogenic shock. In some embodiments, the nuclease inhibitor is metaraminol (+)-bitartrate salt. Metaraminol (+)-bitartrate salt is an adrenergic agonist that acts predominantly at alpha adrenergic receptors and stimulates the release of norepinephrine. It is also an FDA proved drug used primarily as a vasoconstrictor in the treatment of hypotension.

### VII. Nucleases

The present disclosure provides methods and compositions of increasing cfDNA level in a bodily fluid of a subject by inhibiting the activities of nucleases in the bodily fluid. The term “nuclease” refers to any enzyme capable of cleaving the phosphodiester bonds between nucleotides of nucleic acids. Nucleases include, but not limited to DNA nucleases (or deoxyribonucleases, DNases), RNA nucleases (or Ribonucleases, RNases), DNA/RNA nucleases, exonucleases, endonucleases, exo-endonucleases, Benzonase®, a micrococcal nucleases (MNases), transposases, Type I restriction enzymes, Type II restriction enzymes, Type III restriction enzymes, Type IV restriction enzymes, Type V restriction enzymes, Nucleases S1, Nucleases P1, Flap Endonucleases (FEN), Helicase-dependent Nucleases, Mitochondrial Nucleases, CRISPR-associated Nucleases (Cas nucleases), and any functional derivative, fragment or fusion thereof. The methods and compositions described herein can used to inhibit any nuclease enclosed above.

In some embodiments, the nuclease to be targeted is a deoxyribonuclease (DNase). The DNase can be DNase I, DNase II, DNA fragmentation factor subunit beta (DFFB), or any other apoptosis associated DNases. DNase I family includes four types of DNases: DNase I, deoxyribonuclease 1-like 1 (DNase1L1), DNase1L2 and DNase1L3. DNase I works well at neutral pH (6.5-8.0) and requires bivalent ions like calcium (Ca2+) and magnesium (Mg2+) for activation. DNase II family includes three types of DNases: DNase II (α), DNase II β and L-DNase II. DNase II works well at acidic pH and does not require bivalent ions. DFFB, also named caspase-activated DNase (CAD), breaks up the DNA during apoptosis and promotes cell differentiation. Other apoptotic nucleases such as endonuclease G, AIF, topoisomerase II, and cyclophilins can be targeted to inhibit their activities, thereby enhancing cfDNA levels in the bodily fluid samples.

In some instances, a DNase inhibitor described herein specifically inhibits one type of DNase, such as a DNase I family member, more specifically, DNase1L3. In other preferable instances, a DNase inhibitor described herein work against multiple types of DNases. For examples, a DNase inhibitor can reduce or block the activities of both DNase I and DNase II family members, or multiple members in the same DNase family, such as DNase1 and DNase1L3.

Among the many enzymes which catalyze the hydrolysis of DNA, DNASE1 and DNASE1L3 are the two main types of DNA nucleases associated with cfDNA fragmentation patterns (Han et al. Am J Hum Genet. 2020; 106:202-214). DNASE1 and DNASE1L3 share similar catalytical mechanism since both of them belong to DNase I family. For example, both enzymes are endonucleases that require divalent cations (Ca²⁺/Mg²⁺) for activity. However, some structural differences are present between the nucleases, conferring them different properties. DNASE1L3 contains a flexible C-terminal domain (CTD), composed of residues Ser-283 to Ser-305, which increases its ability to bind DNA (McCord et al. Commun Biol. 2022; 5:825). DNASE1L3 can cleave DNA associated with proteins/lipids effectively, whilst DNASE1 prefers protein-free DNA. The different structures suggest that the two nucleases may have different susceptibilities to inhibitors. For example, G-actin can inhibit DNASE1, but not DNASE1L3 (Han, Lo, Trends Genet. 2021; 37:758-770). Currently, there were three reported DNASE1L3 inhibitors including 4-(4,6-dichloro-[1,3,5]-triazin-2-ylamino)-2-(6-hydroxy-3-oxo-3H-xanthen-9-yl)-benzoic acid (DR396), Pontacyl Violet 6R (PV6R), and Fmoc-D-Cha-OH (FDCO) (Yamada et al. Bioorg Med Chem. 2011; 19:168-171; Kolarevic et al. Eur J Med Chem. 2014; 88:101-111). None of the aforementioned inhibitors are FDA-approved.

### A. DNASE1

In some embodiments, the nuclease of interest is a DNase1. DNase I (or Deoxyribonuclease I) is an endonuclease of the DNase family coded by the human gene DNASE1. DNase I is a nuclease that cleaves DNA preferentially at phosphodiester linkages adjacent to a pyrimidine nucleotide, yielding 5′-phosphate-terminated polynucleotides with a free hydroxyl group on position 3′, on average producing tetranucleotides. It acts on single-stranded DNA, double-stranded DNA, and chromatin. In addition to its role as a waste-management endonuclease, it has been suggested to be one of the deoxyribonucleases responsible for DNA fragmentation during apoptosis.

### B. DNASE1L3

In some embodiments, the nuclease of interest is a DNase1L3. DNASE1L3 (also termed deoxyribonuclease 1L3, deoxyribonuclease I like 3, deoxyribonuclease gamma, DNase Y) is an enzyme that is encoded by the DNASE1L3 gene in humans. DNASE1L3 belongs to the family of deoxyribonuclease enzymes that are responsible for degrading DNA. Specifically, DNASE1L3 plays a key role in the breakdown of extracellular DNA, particularly DNA released from dying cells due to apoptosis or necrosis. As disclosed herein, a human DNASE1L3 protein comprises an amino acid sequence at least 80%, 85%, or 90% identity to SEQ ID NO: 1 or comprises an amino acid sequence having at least 95%, 96%, 97%, 98%, or 99% identity to SEQ ID NO: 1. The present disclosure also provides a nucleic acid sequence encoding a human DNASE1L3 protein. The nucleic acid sequence comprises a sequence at least 80%, 85%, or 90% identity to SEQ ID NO: 2 or comprises an amino acid sequence having at least 95%, 96%, 97%, 98%, or 99% identity to SEQ ID NO: 2. The nucleic acid sequence can be codon-optimized to facilitate protein expression in different host systems such as insect cell system or yeast expression system. For example, the nucleic acid sequence can be codon-optimized for human DNASE1L3 expression in an insect cell system. In some embodiments, the codon-optimized nucleic acid sequence comprises a sequence at least 80%, 85%, or 90% identity to SEQ ID NO: 3 or comprises an amino acid sequence having at least 95%, 96%, 97%, 98%, or 99% identity to SEQ ID NO: 3.

### 1. Example Protocols to Obtain Human DNASE1L3

This section describes exemplary protocols for producing recombinant human DNASE1L3 proteins.

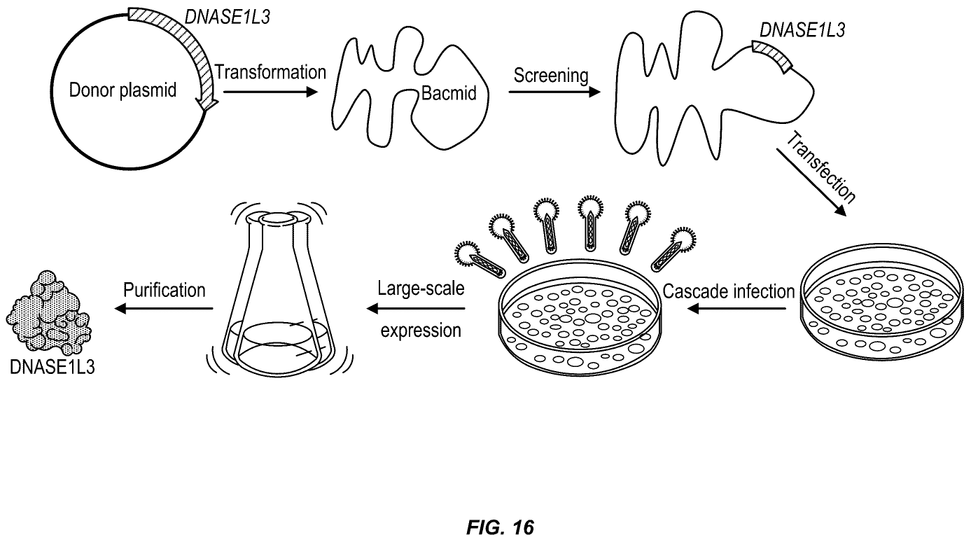

FIG. 16 details the protocol we developed to express human DNASE1L3 proteins. Donor plasmid containing codon-optimized human DNASE1L3 gene was transformed into DH10Bac cells. After white/blue screening, white colonies were picked to extract the bacmid and the extracted bacmid then were transfected into Sf9 cells for P0 baculovirus production. Sf9 cells were collected after 5 days, and the protein expression was confirmed by western blot on the cell lysates. Successful colonies were preserved in glycerol stock in −80° C., and the cell medium containing the P0 virus were filtered, and supplemented with 0.2% BSA, stored in 4° C. protected from light. The medium containing secreted DNASE1L3 was loaded into a strep-tactin XT column. Enriched DNASE1L3 protein was eluted by supplementing 50 mM Biotin into the wash buffer, and further identified by stain as well as western blot using anti-DNASE1L3 antibody (Abcam ab152118).

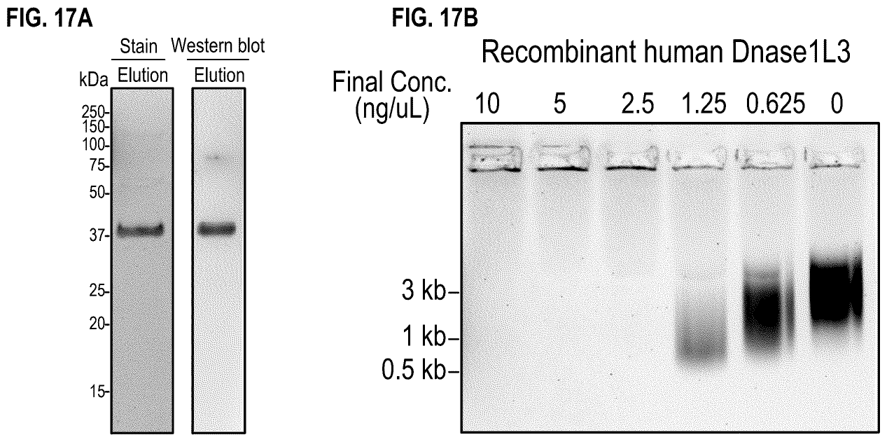

The purified protein was then analyzed by SDS-PAGE and Western blot to confirm its identity (FIGS. 17A and 17B). To assess the functional activity of the recombinant DNASE1L3, an in vitro DNA digestion assay was performed. The purified protein exhibited nuclease activity, as evidenced by its ability to degrade calf thymus DNA in a concentration-dependent manner. This demonstrated the insect cell system was able to properly express and produce biologically active human DNASE1L3 protein.

### 2. Methods for Producing DNASE1L3

In one aspect, the present disclosure provides methods for producing a recombinant DNASE1L3 (e.g., human DNASE1L3). The produced recombinant DNASE1L3 are suitable for in vitro assays, e.g., as described in section III, or for DNase inhibitor screenings, e.g., as described in section IV. In some cases, the DNase inhibitor screenings can be high-throughput inhibitor screenings. Example methods are described below.

In a 1st step, a polynucleotide can encode DNASE1L3 protein (e.g., human DNASE1L3) into a host cell. As described herein As described herein, recombinant human DNASE1L3 is a secreted glycoprotein requiring proper disulfide bond formation, N-glycosylation (or compatible insect-type glycosylation), and a free C-terminus for full enzymatic activity on chromatin and microparticle-associated DNA. Although in principle many host systems may be considered, successful production of active, secreted DNASE1L3 has been reported only in eukaryotic systems capable of complex post-translational modifications and efficient secretion. Suitable eukaryotic hosts include, but are not limited to: (a) insect cells (e.g., Spodoptera frugiperda Sf9, Sf21, or Trichoplusia ni High Five cells infected with baculovirus); (b) mammalian cells (e.g., HEK293, CHO, NS0, BHK, COS); (c) certain yeast and fungal systems engineered for humanized glycosylation (e.g., Pichia pastoris strains with modified glycoforms, GlycoSwitch strains). (d) Prokaryotic systems (e.g., Escherichia coli), standard Saccharomyces cerevisiae, plant cells, and most cell-free systems have been reported to yield misfolded, insoluble, or inactive DNASE1L3 lacking the required post-translational processing and secretion. In the embodiments disclosed herein, recombinant human DNASE1L3 is expressed in an insect cell/baculovirus system, in particular Sf9, Sf21, or High Five cells, which provides high yield, proper secretion, insect-type glycosylation compatible with activity, and scalability suitable for high-throughput screening applications (see FIGS. 16-17).

As disclosed herein, the recombinant DNASE1L3 can be expressed in an insect cell via a baculovirus expression vector. The baculovirus expression vector is a recombinant baculovirus (derived from Autographa californica multicapsid nucleopolyhedrovirus, AcMNPV) in which a heterologous expression cassette has been inserted into the baculoviral genome, typically by replacing the non-essential polyhedrin (polh) gene or the p10 gene with the gene of interest under the transcriptional control of a very strong baculoviral late promoter (e.g., the polyhedrin promoter (pPolh) or the p10 promoter). In some embodiments, the polynucleotide comprises a nucleic acid sequence having at least 80% identity to SEQ ID NO: 2 or SEQ ID NO: 3. In particular embodiments, the polynucleotide comprises a nucleic acid sequence of SEQ ID NO: 2 or SEQ ID NO: 3. The DNASE1L3 is expressed using a baculovirus-insect cell expression system, which provides higher yield, improved purity, and consistent post-translational modifications compared to prior mammalian cell-based methods. A nucleic acid encoding human DNASE1L3 codon-optimized is cloned into a baculovirus transfer vector. The recombinant baculovirus is generated in Sf9 insect cells and used to infect High Five or Sf9 cells for protein expression.

In the embodiments disclosed herein, a nucleic acid encoding human DNASE1L3 (e.g., codon-optimized SEQ ID NO: 3) with its native signal peptide is cloned into a commercially available or custom baculovirus transfer vector (e.g., pFastBac1, pFastBacDual, POET, or derivatives thereof). The transfer vector contains the expression cassette flanked by baculoviral sequences that enable homologous recombination or Tn7-mediated transposition in Escherichia coli (e.g., DH10Bac or DH10MultiBac strains) to generate the recombinant bacmid. The resulting bacmid is transfected into Spodoptera frugiperda Sf9 or Sf21 cells to produce the initial recombinant baculovirus stock (P1/P2), which is subsequently amplified and used at high titre to infect Sf9, Sf21, or Trichoplusia ni High Five cells for large-scale protein expression (see workflow in FIG. 16). This baculovirus-insect cell system provides high-level secretion of properly folded, glycosylated, and fully active human DNASE1L3 (see validation in FIGS. 17A-17B), with yields typically in the range of 1-20 mg/L and superior consistency compared to transient mammalian cell transfection methods previously used for DNASE1L3 (Sisirak et al., Cell 2016; Napirei et al., Biochem J 2005).

In a 2nd step, the recombinant DNASE1L3 can be expressed in an insect cell culture under standard culture conditions. Procedures for recombinant protein expression in an insect cell system are known in the art. (See e.g., Berger et al., Nat Biotechnol 22, 1583-1587, 2004)

In an optional 3rd step, the recombinant DNASE1L3 can be purified from the insect cell culture. Examples of recombinant protein purification include but not limited to, as those described in Fitzgerald et al., Nat Methods 3, 1021-1032, 2006, the entire disclosure of which is herein incorporated by reference. In particular embodiments, the secreted recombinant DNASE1L3 is purified from the culture supernatant using affinity chromatography, ion-exchange chromatography, or size-exclusion chromatography, or combinations thereof. Purity is confirmed by SDS-PAGE and Western blot (see FIG. 17A), and enzymatic activity is validated using a fluorescent probe-based assay (see FIG. 17B).

Unlike prior methods using transient transfection of mammalian cells (e.g., HEK293 or NIH-3T3) that yield crude, low-volume supernatants with inconsistent glycosylation and potential contamination by host nucleases (Napirei et al., Biochem. J., 2005, 389(Pt 2):355-64; Sisirak et al., Cell, 2016, 166(1):88-101), the baculovirus-insect cell system enables scalable production of milligram quantities of functional, homogeneous DNASE1L3, essential for reproducible high-throughput screening.

### VIII. Use for Liquid Biopsy

This section describes how nuclease inhibition strategies can enhance liquid biopsy for analyzing circulating cell-free DNA (cfDNA) in biological fluids. Effective liquid biopsy relies on increasing the amount and stability of cfDNA and accurately measuring its levels. The present disclosure provides methods for boosting cfDNA concentration by preventing its degradation, for assaying cfDNA in samples, and for inhibiting nucleases to maintain cfDNA integrity. Collectively, these methods aim to improve the sensitivity and reliability of liquid biopsy for diagnostic and monitoring purposes.

A. Methods of Increasing cfDNA Level

In one aspect, the present disclosure provides a method of increasing cell-free DNA (cfDNA) level in a bodily fluid of a subject. In some embodiments, the method comprises administering to the subject a therapeutically effective amount of a nuclease inhibitor to inhibit cleavage of the cfDNA in the bodily fluid, thereby increasing the cfDNA level in the subject.

In some embodiments, the subject has been identified for a liquid biopsy to be assayed for cfDNA. A liquid biopsy is a non-invasive or minimally invasive laboratory test or assay alternative to surgical biopsies for disease diagnosis or monitoring through a liquid biological sample. Liquid biopsies can be used for detecting, analyzing and monitoring diseases, such as cancers, in various body effluents such as blood or urine. A liquid biopsy comprises multiple biological matrices such as cell free nucleic acids (e.g., cfDNA), circulating tumor cells (CTCs), and/or exosomes. Liquid biopsies can be also used for real-time monitoring of cancer evolution and treatment. The present disclosure may be used with any liquid biopsy approach known in the art, such as those described in the following references, each of which are incorporated in their entireties by reference: Yadav et al., “Detection of circulating tumour cells in colorectal cancer: Emerging techniques and clinical implications,” World J Clin Oncol. 2021 Dec. 24; 12(12):1169-1181; Bunduc et al., “Exosomes as prognostic biomarkers in pancreatic ductal adenocarcinoma a systematic review and meta-analysis,” Transl Res. 2022 Jan. 20: S1931-5244; Takami H, et al., “Advances in Molecular Profiling and Developing Clinical Trials of CNS Germ Cell Tumors: Present and Future Directions,” Curr Oncol Rep. 2022 Jan. 20; and Li et al., “Liquid biopsy in lung cancer: significance in diagnostics, prediction, and treatment monitoring,” Mol Cancer. 2022 Jan. 20; 21(1):25; Underwood et al., “Liquid biopsy for cancer: review and implications for the radiologist,” Radiology, Nov. 19, 2019.

As disclosed herein, the bodily fluid to be examined for the cfDNA level can be, but not limited to, blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal fluid, discharge fluid from the nipple, aspiration fluid from different parts of the body (e.g., thyroid, breast), intraocular fluids (e.g., the aqueous humor), amniotic fluid, aqueous humor, ascites, bone marrow fluid, lymphatic fluid, synovial fluid, interstitial fluid, prostate fluid, semen, mucus, gastric acid, bile, pus, cerumen, breast milk, cowper's fluid or pre-ejaculatory fluid, female ejaculate, hair oil, cyst fluid, dialysis fluid, pericardial fluid, chyme, chyle, menses, sebum, vomit, mucosal secretion, stool water, pancreatic juice, lavage fluids from sinus cavities, bronchopulmonary aspirates, blastocoel fluid, urinary tract secretions, urethral secretions, bladder secretions, prostate secretions, vesical secretions, meconium, and umbilical cord fluid. In some embodiments, the bodily fluid comprises blood, serum, and/or plasma. In a particular embodiment, the bodily fluid is a blood sample.

In some instances, the nuclease inhibitor can be an antibiotic. In some embodiments, the antibiotic is a cephalosporin, such as ceforanide or a derivative thereof. In other instances, the nuclease inhibitor is a non-antibiotic molecule that binds to a catalytic pocket of the nuclease. As disclosed herein, the nuclease inhibitor can block or reduce the function of one particular DNase, or two or more DNases, such as three, four, five, six, seven, eight, nine, or ten DNases as described herein. In particular embodiments, the nuclease inhibitor inhibits the activities of DNase I, DNase1L3, and/or DFFB.

As disclosed herein, the nuclease inhibitor can be administered orally, intravenously, intradermally, intramuscularly, intraperitoneally, subcutaneously, intranasally, epidurally, sublingually, intracerebrally, intravaginally, trans-dermally, rectally, by inhalation, or topical administration. In particular embodiments, the nuclease inhibitor is administered orally.

The term “cfDNA level” refers to the concentration or amount of cfDNA molecules in a bodily fluid of a subject. In some embodiments, the cfDNA level can be expressed as nanograms per milliliter (ng/mL) or genome equivalents per milliliter (GE/mL). The cfDNA level can be quantified by various assays such as qPCR, fluorometry (QuBit), sequencing, etc.

As disclosed herein, the therapeutically effective amount of the nuclease inhibitor is about 1-50 uM per kilogram body weight. In some embodiments, the therapeutically effective amount of the nuclease inhibitor is about 10-30 uM per kilogram body weight. In some embodiments, the therapeutically effective amount of the nuclease inhibitor is about 20 uM per kilogram body weight.

In some embodiments, the therapeutically effective amount of the nuclease inhibitor is determined by the following parameters: (i) Volume of distribution (VD)=0.13 to 0.4 L/kg (R. S. Vardanyan, V. J. Hruby, in Synthesis of Essential Drugs, 2006); (ii) Plasma half-life (t½)=2.5 to 3.5 hours (R. S. Vardanyan, V. J. Hruby, in Synthesis of Essential Drugs, 2006); and (iii) Clearance (CL)=35 to 55 mL per min. The effective dose of a nuclease inhibitor to a subject can be determined through the following steps: (1) determining the subject's VD range using a formula: VD range=VD×the subject's body weight; (2) determining the steady-state concentration (SSC) required to achieve a required IC50; (3) determining the total amount of drug in the body at steady-state using a formula: Total amount of drug=SSC×VD range; and (4) determining the dosing regimen to achieve steady-state using a formula: Steady-state dose (SSD)=SSC×CL/(1−e\hat{ }(−ln(2)×(τ/t½))), where τ=dosing interval (e.g., 8 hours for 3 times every day).

For example, to determine the effective dose of a nuclease inhibitor to a subject with a body weight of about 70 kg, the following steps can be conducted:
1. Determine the VD range 0.13 to 0.4 L/kg×70 kg=9.1 to 28 L
2. Determine the steady-state concentration (SSC) required to achieve the IC50 of 20 uM:

$$SSC = IC 50 = 20 uM$$

3. Determine the total amount of drug in the body at steady-state:

$$Total amount of drug = SSC \times VD = 20 uM \times ( 9.1 to 28 L ) = 182 to 260 mg$$

4. Determine the dosing regimen to achieve steady-state:

$$Clearance ( Cl ) = 35 to 55 mL per min = 0.035 to 0.055 L per min$$

$$Half - life ( t 1 / 2 ) = 2.5 to 3.5 hours$$

$$SSD = 20 uM \times ( 0.035 to 0.055 L / min ) / ( 1 - e \hat{} ( - ln ( 2 ) \times ( 8 / 2.5 ) to ( 8 / 3.5 ) ) ) = 24 to 53 mg per every 8 hours .$$

Therefore, to achieve steady-state concentration of 20 uM with the given pharmacokinetics parameters by R. S. Vardanyan, V. J. Hruby, in Synthesis of Essential Drugs, 2006, the total daily dose of a nuclease inhibitor to the subject is about 72 to 159 mg. If divided into 3 doses, each dose is about 24 to 53 mg given every 8 hours for a subject with a body weight of about 70 kg. The exact dose is subjected to individual response and effectiveness drug monitoring.

Without desiring to be bound by theory, the nuclease inhibitor capable of inhibiting deoxyribonucleases (DNases) when administered to a subject may transiently increase the concentration of cfDNA found in the bodily fluids of a subject, particularly blood or urine, by interfering with the activity of one or more DNases in bodily fluids of the subject. In particular, such nuclease inhibitors may inhibit the activity of DNase1 and/or DNase1L3 located in the bloodstream which typically act upon cfDNA, degrading it into progressively shorter fragments. Accordingly, the nuclease inhibitor may enhance the amount of cfDNA subsequently recovered by a liquid biopsy (e.g., a blood sample or a urine sample) for use in downstream applications such as sequencing.

In some embodiments, administration of a therapeutically effective amount of a nuclease inhibitor capable of inhibiting one or more DNases results in a concentration of cfDNA that is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, or more than 1000-fold relative to the concentration of cfDNA before the nuclease inhibitor capable of inhibiting one or more DNases is administered.

In some embodiments, the effective amount of administered nuclease inhibitor capable of inhibiting one or more DNases is characterized by the degree to which it limits biological processes that normally result in the removal of cfDNA from one or more bodily fluid samples. In some embodiments, the effective amount of administered nuclease inhibitor capable of inhibiting one or more DNases the activity of DNases upon cfDNA in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the activity of the deoxyribonucleases in the absence of the nuclease inhibitor.

In some embodiments, the subject is a human. In some embodiments, the subject is pregnant, and thereby the circulating cfDNAs in the subject's bodily fluids comprise a mixture of fetal and subject's DNAs. In some embodiments, the subject is a human patient that has, is suspected of having, or is at risk of having a disease associated with the presence of cfDNA in one or more bodily fluids. In some embodiments, the disease is cancer, a premalignant condition, an inflammatory disease, an immune disease, an autoimmune disease or disorder, a cardiovascular disease or disorder, neurological disease or disorder, infectious disease, or pain.

In some embodiments, the disease is cancer. In some embodiments, the cancer is selected from the group consisting of colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, gallbladder cancer, brain cancer, peritoneal cancer, lymphoma, leukemia, multiple myeloma, neuroblastoma, osteosarcoma, head and neck cancer, oral cancer, nasopharyngeal cancer, skin cancer and soft tissue sarcoma.

B. Methods of Assaying cfDNA

In another aspect, the method of administering to a subject a therapeutically effective amount of a nuclease inhibitor can be applied to a method of assaying cfDNA from a bodily fluid of the subject. In some embodiments, the method of assaying cfDNA from a bodily fluid of a subject comprises (a) increasing cfDNA level in the bodily fluid of the subject by performing the method described above; and (b) assaying the cfDNA.

In some embodiments, the method of assaying cfDNA comprises the following steps: (a) increasing cfDNA level in the bodily fluid of the subject by administering to the subject a therapeutically effective amount of a nuclease inhibitor; (b) obtain a bodily fluid sample from the subject, wherein the bodily fluid sample comprises the cfDNA; and (c) assaying the cfDNA from the bodily fluid sample.

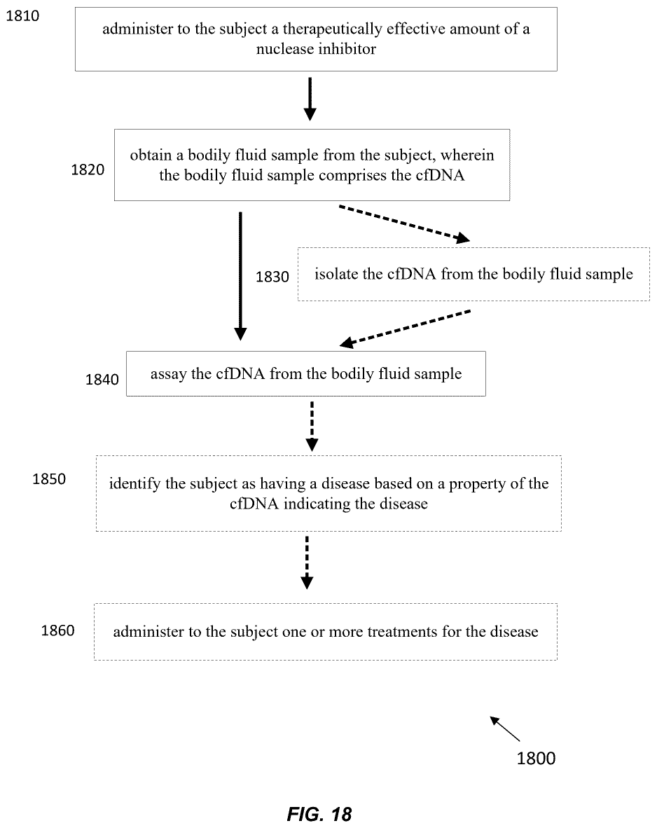

FIG. 18 is a flowchart illustrating a method 1800 of assaying cfDNA from a bodily fluid of a subject according to embodiments of the present invention.

At block 1810, a therapeutically effective amount of a nuclease inhibitor is administered to the subject, wherein the nuclease inhibitor binds to a nuclease and inhibits cleavage of the cfDNA in the bodily fluid, thereby increasing the cfDNA level in the subject. In some embodiments, the subject has been identified for a liquid biopsy to be assayed for cfDNA. This step is described in detail above.

At block 1820, a bodily fluid sample containing cfDNA is obtained from the subject. The bodily fluid sample may also be obtained using any available biopsy technique, including a needle-biopsy technique (including techniques such as fine-needle aspiration, core needle biopsy, vacuum-assisted biopsy, or image-guided biopsy), an endoscopic biopsy, a skin biopsy (e.g., a shave biopsy, punch biopsy, incisional biopsy, or excisional biopsy), a bone marrow biopsy, or a surgical biopsy. In some embodiments, cfDNA can be assayed in the bodily fluid sample. In some embodiments, the bodily fluid sample is obtained at least 1 minute, at least 2 minutes, at least 3 minutes, at least 4 minutes, at least 5 minutes, at least 10 minutes, at least 15 minutes, at least 30 minutes, at least 1 hour, at least 2 hours, at least 3 hours, at least 4 hours, at least 5 hours, at least 10 hours, at least 20 hours, or at least 24 hours after the therapeutically effective amount of nuclease inhibitor is administered to the subject. In some embodiments, the bodily fluid sample is obtained more than 24 hours after the administering step, such as 36, 48, 72 hours after the nuclease inhibitor administering. In some embodiments, the bodily fluid sample is obtained between 1 minute to 72 hours after the administering step. In some embodiments, the bodily fluid sample is obtained between 1 minute to 24 hours after the administering step.

In some embodiments, the bodily fluid sample can be freshly collected for cfDNA assessment. In some embodiments, the bodily fluid sample can be stored (e.g. refrigerated, or frozen) in certain time before performing cfDNA assessment. In some embodiments, the sample can be a processed, such as centrifugation, filtration, sonication, homogenization, heating, freeze-thawing, contact with a preservative (e.g., an anticoagulant or nuclease inhibitor), and the like. In some embodiments, the sample can be treated with chemical and/or biological reagents. Chemical and/or biological reagents can be used to protect and/or maintain the stability of the cfDNA during processing and/or storage. In some embodiments, chemical and/or biological reagents can be used to release cfDNA from other components of the sample (e.g., from protein aggregates). Those skilled in the art are aware of methods and processes for processing and storing samples for cfDNA analysis.

In some embodiments, cfDNA is isolated from the bodily fluid sample prior to assaying. Block 1830 is an optional step that cfDNA is isolated from the bodily fluid sample prior to assaying. In some embodiments, the cfDNA can first be amplified by PCR to amplify the material to be analyzed. The isolated cfDNA and/or amplification product thereof can be isolated from the enzyme, primer, or buffer components before sequencing or further analysis. Methods for isolating nucleic acids are well known in the art.

At block 1840, the cfDNA from the bodily fluid sample is analyzed. The cfDNA may be assayed by any means known in the art, such as but not limited to sequencing, amplification, hybridization, gel electrophoresis, chromatography, immunoassay, enzyme immunoassay (EIA), enzyme-linked immunosorbent assay (ELISA), enzyme-linked oligonucleotide assay (ELONA), affinity isolation, immunoprecipitation, Western blot, flow cytometry, and any combination thereof. In some embodiments, assaying the enriched cfDNA from the bodily fluid sample can be used to identify one or more diseases, and/or one or more candidate drugs for therapeutic intervention.

In some embodiments, the cfDNA is assayed at least 1 minute, at least 2 minutes, at least 3 minutes, at least 4 minutes, at least 5 minutes, at least 10 minutes, at least 15 minutes, at least 30 minutes, at least 1 hour, at least 2 hours, at least 3 hours, at least 4 hours, at least 5 hours, at least 10 hours, at least 20 hours, or at least 24 hours after the therapeutically effective amount of nuclease inhibitor is administered to the subject. In some embodiments, the cfDNA is assayed more than 24 hours after the administering step, such as 36, 48, 72 hours after the nuclease inhibitor administering. In some embodiments, the cfDNA is assayed between 1 minute to 72 hours after the administering step. In some embodiments, the cfDNA is assayed between 1 minute to 24 hours after the administering step. In some embodiments, the cfDNA is assayed between 1 minute to 24 hours after a stored bodily fluid sample ready to be assayed, such as a stored sample thaw from a frozen status.

Block 1850 can identify the subject as having a disease based on the cfDNA assay result. In some embodiments, the method of assaying cfDNA further comprises a step of (d) identifying the subject as having a disease based on a property of the cfDNA indicating the disease. The property can be, but not limited to, copy numbers, sizes, end motifs, jagged ends, sequence variations (mutations), methylation changes, nucleosome signal patterns, preferred ending positions, and nucleosome footprints. The property of the cfDNA can be determined using assay results detected in the assaying step. For example, assaying (e.g., sequencing, amplifying) cfDNA identifies the presence of one or more mutations or biomarkers in the cfDNA which are indicative of a disease.

In some embodiments, the subject is identified as having a disease when sequencing of cfDNA results in the detection of one or more mutations that are indicative of the disease. In some embodiments, the disease is cancer, a premalignant condition, an inflammatory disease, an immune disease, an autoimmune disease or disorder, a cardiovascular disease or disorder, neurological disease or disorder, infectious disease, or pain.

In some embodiments, the subject is identified as having cancer when sequencing of cfDNA results in the detection of one or more cancer antigens or biomarkers that are indicative of the cancer. As disclosed herein, the cancer may include, though is not limited to, colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, gallbladder cancer, brain cancer, peritoneal cancer, lymphoma, leukemia, multiple myeloma, neuroblastoma, osteosarcoma, head and neck cancer, oral cancer, nasopharyngeal cancer, skin cancer and soft tissue sarcoma.

Block 1860 is an optional step of the method of assaying cfDNA that the subject is administered to one or more treatments for the disease. In some embodiments, the method of assaying cfDNA further comprises a step of (e) administering to the subject one or more treatments for the disease.

Therapies can be selected from an administration of an effective amount of a chemotherapeutic compound, an effective amount of an immunotherapeutic compound, and/or an effective amount of a hormone therapeutic compound to kill cancer cells or limit their proliferation. In some embodiments, a targeted therapy is selected that is intended to specifically kill or limit the proliferation of cancer cells by interfering with one or more biological process required by those cells for survival. In some embodiments, therapies are selected from radiation therapy, radiofrequency ablation, cryoablation, and/or surgical intervention to remove cancerous or pre-cancerous tissue. In some embodiments, selected therapies include transplantation of healthy donor cells and/or organs to the patient, such as to replace diseased cells and/or organs.

### C. Methods of Inhibiting Nuclease Activities

As disclosed herein, the method of administering to a subject a therapeutically effective amount of a nuclease inhibitor can be applied to a method of inhibiting activity of a nuclease in the subject. In some embodiments, the method of inhibiting activity of a nuclease in a subject comprises administering to the subject a therapeutically effective amount of a nuclease inhibitor that binds to the nuclease, thereby inhibiting the nuclease activity in the subject.

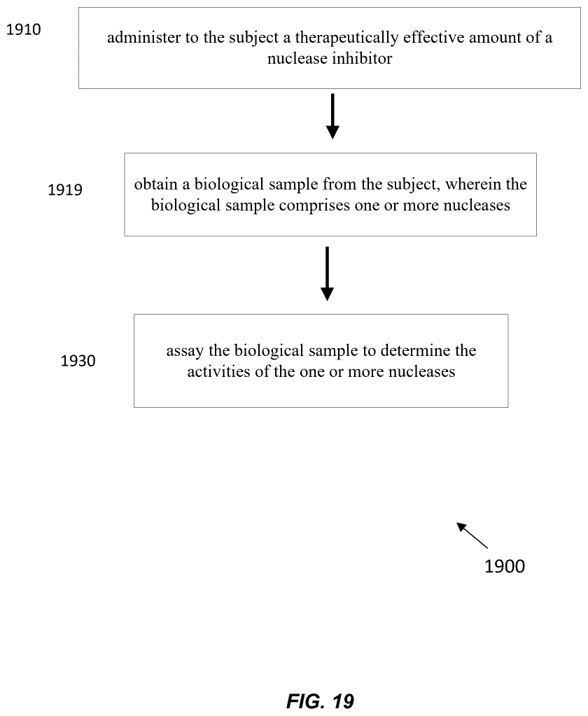

FIG. 19 is a flowchart illustrating a method 1900 of inhibiting activity of a nuclease in a subject according to embodiments of the present invention.

At block 1910, a therapeutically effective amount of a nuclease inhibitor is administered to the subject. As disclosed herein, the nuclease inhibitor can be either an antibiotic or a non-antibiotic molecule. In some instances, the nuclease inhibitor is an antibiotic such as a cephalosporin antibiotic. In other instances, the nuclease inhibitor is a non-antibiotic molecule such as a nucleotide, protein, or synthetic organic and inorganic compound that can inhibit a nuclease's activity. In some embodiments, the nuclease inhibitor is an FDA-approved drug.

In some embodiments, the nuclease inhibitor is an antibiotic drug. In some embodiments, the nuclease inhibitor is an antibiotic cephalosporin. In some embodiments, the antibiotic is selected from the group consisting of ceforanide, cefuroxime sodium, cefamandole sodium, cefaclor, cefdinir, sulfadiazine, sulphapyridine, and a derivative thereof. In a particular embodiment, the nuclease inhibitor is antibiotic ceforanide. In some embodiments, the antibiotic drug is an FDA-approved drug. In some embodiments, the nuclease inhibitor is a non-antibiotic molecule such as synephrine, isoprenaline, and metaraminol (+)-bitartrate salt.

As disclosed herein, the nuclease inhibitor can be administered orally, intravenously, intradermally, intramuscularly, intraperitoneally, subcutaneously, intranasally, epidurally, sublingually, intracerebrally, intravaginally, trans-dermally, rectally, by inhalation, or topical administration. In particular embodiments, the nuclease inhibitor is administered orally.

At block 1920, a biological sample containing one or more nucleases is obtained from the subject. The biological sample can be a biopsy sample such as a tissue, fluid, or cell sample from the subject. The biological sample can be obtained through a needle-biopsy technique (including techniques such as fine-needle aspiration, core needle biopsy, vacuum-assisted biopsy, or image-guided biopsy), an endoscopic biopsy, a skin biopsy (e.g., a shave biopsy, punch biopsy, incisional biopsy, or excisional biopsy), a bone marrow biopsy, or a surgical biopsy. The biological sample can be freshly collected for nuclease assessment, or stored (e.g. refrigerated, or frozen) in certain time before performing the nuclease assessment. In some embodiments, the biological sample can be processed or treated with chemical and/or biological reagents for the nuclease assessment.

At block 1930, the biological sample is analyzed to access the activities of the one or more nucleases. Those skilled in the art are aware of methods and processes for nuclease activity assessment, such as radioactive isotope-based gel electrophoresis, or the fluorescence-based approach as described above in the section III.

The methods and compositions described herein can be used to inhibit any kinds of nucleases in a subject. In some embodiments, the method is used to inhibit the activity of one or more deoxyribonucleases (DNases). The one or more DNases can be a DNase I. a DNase II, DFFB, and/or or any other apoptosis associated DNases. DNase I family includes DNase I, DNase1L1, DNase1L2 and DNase1L3. DNase II family includes DNase II (a), DNase II β and L-DNase II. In some embodiments, the DNase is deoxyribonuclease 1 (DNase1), deoxyribonuclease 1-like 1 (DNase1L1), DNase1L2, DNase1L3, or DNA fragmentation factor subunit beta (DFFB) nuclease.

In some embodiments, the method described herein results in inhibiting the function of one particular DNase. In other embodiments, the method is used to inhibit the activities of two or more DNases, such as three, four, five, six, seven DNases as described herein. In particular embodiments, the method results in inhibiting the activities of DNase I and/or DNase1L3.

In some embodiments, after administering a therapeutically effective amount of a nuclease inhibitor to a subject, the activity of the nuclease of interest is inhibited by about 5%, 10%, 15%, 20%, 25%, 30%, 35%, 40%, 45%, 50%, 55%, 60%, 65%, 70%, 75%, 80%, 85%, 90%, 95%, 99%, or 100%, as compared to the nuclease activity in the absence of the nuclease inhibitor. In some embodiments, the subject has been identified for a liquid biopsy to be assayed for cfDNA.

In some embodiments, inhibiting the nuclease activity results in optimizing the level and/or size of cfDNA to be assayed in the liquid biopsy.

### IX. Kits

In another aspect, the present disclosure provides a kit for carrying out the method described herein. In some embodiments, the kit comprises a reagent comprising a nuclease inhibitor of the present disclosure described herein. The kits are useful for inhibiting a nuclease activity, for increasing cfDNA level in a bodily fluid, for assaying cfDNA from a bodily fluid, for diagnosing a disease, and/or for treating a disease of a subject. In some embodiments, the disease is cancer. Some non-limiting examples of which include breast cancer, ovarian cancer, cervical cancer, prostate cancer, pancreatic cancer, colorectal cancer, gastric cancer, lung cancer, skin cancer, liver cancer, brain cancer, eye cancer, soft tissue cancer, renal cancer, bladder cancer, head and neck cancer, mesothelioma, acute leukemia, chronic leukemia, medulloblastoma, multiple myeloma, sarcoma, and any other cancer described herein, including a combination thereof.

In another aspect, the present disclosure provides a kit for assaying DNase activity or screening nuclease inhibitors. In some embodiments, the kit (a) a DNase and (b) a fluorescent probe. In some embodiments, the DNase is a recombinant human DNASE1L3 described herein.

In some embodiments, the fluorescent probe comprises a single stranded DNA oligonucleotide linked with a fluorophore at the 5 terminus and a quencher at the 3 terminus, and without an internal quencher. In some embodiments, the fluorophore is FAM and the quencher is IABkFQ. As disclosed herein, the kit may further comprise a reaction buffer comprising divalent cations Ca²⁺ and Mg²⁺.

Materials and reagents to carry out the various methods of the present disclosure can be provided in kits to facilitate execution of the methods. As used herein, the term “kit” includes a combination of articles that facilitates a process, assay, analysis, or manipulation. In particular, kits of the present disclosure find utility in a wide range of applications including, for example, diagnostics, prognostics, therapy, and the like.

Kits can contain chemical reagents as well as other components. In some embodiments, the kit comprises a reagent comprising a nuclease inhibitor and a pharmaceutically acceptable adjuvant. In the present application, the term “pharmaceutically acceptable adjuvant” generally comprises pharmaceutically acceptable carriers, excipients, or stabilizers, which are nontoxic for the cells or mammals that are exposed to them at the dose and concentration used. Generally, the physiologically acceptable carrier is a PH buffered aqueous solution. Examples of the physiologically acceptable carrier may comprise: buffers, such as phosphate, citrate, and other organic acids; antioxidants, including ascorbic acid; low-molecular-weight (less than about 10 residues) polypeptides, and proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrin; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. In some embodiments, the pharmaceutically acceptable adjuvant comprises divalent cations such as Ca2+ and/or Mg²⁺. In some embodiments, the pharmaceutically acceptable adjuvant is a PH buffered aqueous solution, optionally with a PH value between about 6.0 to about 8.0.

In addition, the kits of the present disclosure can include, without limitation, instructions to the kit user, apparatus and reagents for sample collection and/or purification, apparatus and reagents for product collection and/or purification, apparatus and reagents for administering one or more nuclease inhibitors (e.g., antibiotics) or other composition(s) of the present disclosure, apparatus and reagents for detecting and determining quantity and quality of the cell-free DNAs (cfDNAs) in a bodily fluid, sample tubes, holders, trays, racks, dishes, plates, solutions, buffers or other chemical reagents, suitable samples to be used for standardization, normalization, and/or control samples. Kits of the present disclosure can also be packaged for convenient storage and safe shipping, for example, in a box having a lid. For instance, the kits may be stored and shipped at room temperature, on wet ice or with cold packs, or frozen in the vapor phase of liquid nitrogen or in dry ice.

### X. Example Use Cases

This section provides illustrative use cases demonstrating the practical applications of the methods and compositions described herein. The following subsections present representative example assays that showcase various experimental approaches, highlight key molecular markers relevant to the detection and quantification of cell-free DNA (cfDNA), and summarize typical results obtained using these methodologies. Together, these examples underscore the versatility and effectiveness of the disclosed techniques across a range of diagnostic and research scenarios, offering concrete guidance for their implementation in laboratory and clinical settings.

### A. Example Assays

Various techniques can be used for such analysis in any of the methods described in the present disclosure. For example, the analysis can be performed using sequencing, such as massively parallel sequencing, targeted sequencing, and single molecule sequencing (e.g., using a nanopore or using real-time single molecule sequencing (e.g., from Pacific Biosciences)). Example PCR techniques include real-time PCR and digital PCR (e.g., droplet digital PCR). The analysis can include the physical steps of performing such assays and receiving of the measurement data obtained from such assays, or may just include receiving the measurement data.

Analyzing a cell-free DNA molecule can includes determining a genomic position in a reference genome corresponding to at least one end of the cell-free DNA molecule. For example, one or more sequence reads of a DNA molecule (e.g., paired reads at the ends or a read for the entire molecule) can be aligned to the reference genome using any of various alignments techniques as will be appreciated by the skilled person. The alignment can be to some or all of the reference genome. As another example, probe-based techniques can identify a DNA molecule as being from a particular position, e.g., by emitting a particular color for a particular probe that corresponds to a particular genomic position. The position determination can be to some or all of the reference genome, e.g., if only part of the genome is being analyzed. As examples, the amount of the genome analyzed can be greater than 0.01%, 0.1%, 1%, 5%, 10%, or 50%. Such an analysis may be performed for other methods described herein.

As with other methods described herein, analyzing the plurality of cell-free DNA molecules can includes measuring a size of the cell-free DNA molecule. The measurement can be performed in various ways, e.g., using physical separation (such as electrophoresis) and/or sequencing (such as whole molecule sequencing or alignment using paired-end reads).

### B. Example Markers

Various markers can be identified through the assays described above, such as copy numbers, sizes, end motifs, jagged ends, sequence variations (mutations), methylation changes, nucleosome signal patterns, preferred ending positions, and nucleosome footprints.

For example, we can detect copy number aberrations from a cell-free DNA sample by counting analysis. The aberration of a region can be determined by counting an amount of DNA fragments (molecules) that are derived from the region. As examples, the amount can be a number of DNA fragments, a number of bases to which a DNA fragment overlapped, or other measure of DNA fragments in a region. The amount of DNA fragments for the region can be determined by sequencing the DNA fragments to obtain sequence reads and aligning the sequence reads to a reference genome. In one embodiment, the amount of sequence reads for the region can be compared to the amount of sequence reads for another region so as to determine overrepresentation (amplification) or underrepresentation (deletion). In another embodiment, the amount of sequence reads can be determined for one haplotype and compared to the amount of sequence reads for another haplotype. Further details of counting analysis to identify aberrant regions is described in U.S. Patent Publication No. 2009/0029377 entitled “Diagnosing fetal chromosomal aneuploidy using massively parallel genomic sequencing” by Lo et al. filed on Jul. 23, 2028; and U.S. Patent Publication No. 2016/0201142 entitled “Using Size and Number Aberrations in Plasma DNA For Detecting Cancer” by Lo et al, filed on Jan. 12, 2016, the disclosures of which are incorporated by reference in their entirety for all purposes.

We can also use a size-based analysis to perform a prenatal diagnosis of a sequence imbalance (e.g. a fetal chromosomal aneuploidy) in a biological sample obtained from a pregnant female subject. For example, a size distribution of fragments of nucleic acid molecules for an at-risk chromosome can be used to determine a fetal chromosomal aneuploidy. The size-based analysis can be also used for cancer diagnosis. Some embodiments can also detect other sequence imbalances, such as a sequence imbalance in the biological sample (containing mother and fetal DNA, or containing body and cancer DNA), where the imbalance is relative to a genotype, mutation status, or haplotype of the mother. Such an imbalance can be determined via a size distribution of fragments (nucleic acid molecules) corresponding to a particular sequence relative to a size distribution to be expected if the sample were purely from the mother, and not from the fetus and mother, or from a healthy subject. A shift (e.g. to a smaller size distribution) can signify an imbalance in certain circumstances. Details of size-based analysis are described in U.S. Patent Publication No. 2011/0276277 entitled “Size-Based Genomic Analysis” by Lo et al. filed Nov. 5, 2010 (focused on fetal DNA analysis), U.S. Patent Publication No. 2013/0237431 entitled “Size-Based Genomic Analysis of Fetal DNA Fraction in Maternal Plasma” by Lo et al. filed Mar. 7, 2013 (focused on fetal or cancer DNA analysis), and U.S. Patent Publication No. 2019/0130065 entitled “Using Nucleic Acid Size Range For Noninvasive Prenatal Testing And Cancer Detection” by Lo et al. filed Nov. 1, 2018 (focused on cancer DNA analysis), the contents of which are incorporated herein by reference for all purposes.

The present disclosure also provides techniques for measuring quantities (e.g., relative frequencies) of sequence end motifs of cell-free DNA fragments in a biological sample from a subject for measuring a property of the sample (e.g., fractional concentration of clinically relevant DNA) and/or determining a condition of the subject based on such measurements. An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments. Details of end motif analysis are described in U.S. Patent Publication No. 2020/0199656 entitled “Cell-Free DNA End Characteristics” by Lo et al. filed Dec. 19, 2019, the contents of which are incorporated herein by reference for all purposes.

The present disclosure further provides techniques for jagged end analysis. Double-stranded cell-free DNA fragments may often have two strands that are not exactly complementary to each other. One strand may extend beyond the other strand, creating an overhang. These overhangs are often repaired to form blunt ends in analysis. However, the “jagged ends” created by these overhangs may be useful in analyzing biological samples. As an example, jagged ends in cell-free DNA from a urine sample may be used to diagnose or detect a condition noninvasively and accurately. The degree of jagged ends, which may be the quantity or the length of jagged ends, in a sample may reflect the level of a condition in an individual. For example, the degree of jagged ends may be related to a disease (e.g., cancer), a disorder, a pregnancy-related condition, or a transplant condition. Details of jagged end analysis are described in U.S. Patent Publication No. 2020/0056245 entitled “Cell-Free DNA Damage Analysis and Its Clinical Applications” by Lo et al., filed Jul. 23, 2019, and U.S. Patent Publication No. 2022/0177971-A1 entitled “Methods Using Characteristics of Urinary and Other DNA”, filed Dec. 7, 2021, the contents of which are incorporated herein by reference for all purposes.

The present disclosure further provides techniques for sequence variation (mutation) analysis. Details of sequence variation (mutation) analysis are described in U.S. Patent Publication No. 2014/0100121 entitled “A Method of Measuring a Fractional Concentration Of Tumor DNA”, filed Mar. 13, 2013, and U.S. Patent Publication No. 2017/0073774-A1 entitled “Detecting Mutations For Cancer Screening”, filed Nov. 28, 2016, the contents of which are incorporated herein by reference for all purposes.

The present disclosure further provides techniques for methylation analysis. Details of methylation analysis are described in WO 2014/043763, entitled “Non-Invasive Determination of Methylome of Fetus Or Tumor From Plasma”, filed Sep. 20, 2013, the contents of which are incorporated herein by reference for all purposes.

The present disclosure further provides techniques for nucleosome signal pattern analysis. Details of nucleosome signal pattern analysis are described in U.S. patent application Ser. No. 18/883,637, entitled “Uses of Cell-Free DNA Fragmentation Patterns Associated With Epigenetic Modifications”, filed Sep. 12, 2024, the contents of which are incorporated herein by reference for all purposes.

### C. Example Results

The detection of these molecular markers described above can be useful for the screening, detection, monitoring, management, and prognostication of cancer patients, or patients with other diseases or disorders. The exemplary output results include, but not limited to, level of cancer, aneuploidy (copy number) for tumor or fetal, and fraction of clinically-relevant DNA (e.g., tumor, fetal, or transplant).

For example, the assays described herein can be used to detect fetal inheritance, as detailed in U.S. Patent Publication No. 2011/0105353, entitled “Fetal Genomic Analysis from A Maternal Biological Sample, filed Nov. 5, 2010, and U.S. Patent Publication No. 2017/0029900 entitled “Methylation Pattern Analysis Of Haplotypes In Tissues In A DNA Mixture”, filed Jul. 20, 2016 (focused on methylation pattern analysis), the contents of which are incorporated herein by reference for all purposes.

We can also use the assays described herein to measure levels of cancer, as detailed in U.S. Patent Publication No. 2014/0100121, entitled “A Method Of Measuring A Fractional Concentration Of Tumor DNA”, filed Mar. 13, 2013, and PCT Patent Publication No. WO 2014/043763 entitled “Non-Invasive Determination Of Methylome Of Fetus Or Tumor From Plasma”, filed Sep. 20, 2013, the contents of which are incorporated herein by reference for all purposes.

We can also use the assays described herein to measure fractional concentration, as detailed in U.S. Patent Publication No. 2013/0237431 entitled “Size-Based Analysis Of Fetal Or Tumor DNA Fraction In Plasma”, filed Mar. 7, 2013, and US Patent Publication No. 2020/0199656 entitled “Cell-Free DNA End Characteristics”, filed Dec. 19, 2019, the contents of which are incorporated herein by reference for all purposes.

We can further use the assays described herein to measure copy number (sequence imbalance), as detailed in U.S. Patent Publication No. 2009/0029377 entitled “Diagnosing Fetal Chromosomal Aneuploidy Using Massively Parallel Genomic Sequencing”, filed Jul. 23, 2008, the contents of which are incorporated herein by reference for all purposes.

### XI. Treatments and Further Screening

Responsive to a classification of a pathology or a fractional concentration of clinically-relevant DNA, various actions might be performed, e.g., physical screening steps or treatment(s).

### A. Further Screening Modalities

Based on any classification, e.g., regarding a pathology or fractional concentration of clinically-relevant DNA, the subject can be referred for one or more additional screening modalities, e.g. biopsies (tissue or cell-free, such as liquid or stool) or imaging such as using chest X ray, ultrasound, computed tomography, magnetic resonance imaging, or positron emission tomography. Such screening may be performed for cancer. In this manner, an individual may only be subjected to such screening when (responsive to) there is a high likelihood of the pathology being present, thereby reducing costs, side effects (e.g., radiation exposure), time expenditure of doctor and patients, etc. Additionally, the classification of a pathology (e.g., detection, stage, etc.) can be used to determine a schedule for performing screening modalities, e.g., specifying a frequency for performing the screening modality. Further screening can be performed within a specified amount of time from when the classification is determined, e.g., one day, one week, or one month. The one or more additional screening modalities can be for a particular cancer type, e.g., a particular tissue type., such as imaging a particular organ.

### B. Treatment Selection

Various embodiments of the present disclosure can accurately predict disease relapse, occurrence, and/or severity thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, transplantation, hyperthermia, photodynamic therapy, gene therapy, cell therapy, antibiotics, histotripsy, sound waves, cryoablation, radiofrequency ablation, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Various embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### XII. Example Systems

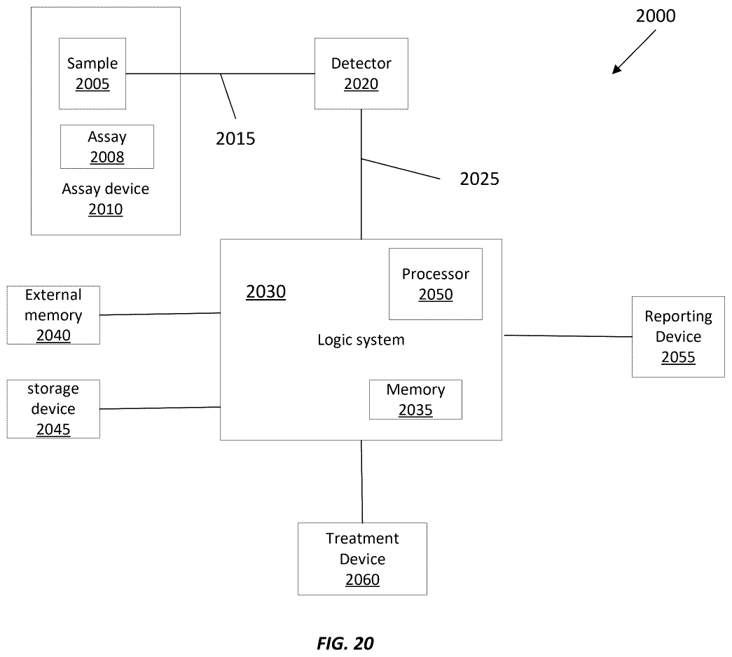

FIG. 20 illustrates a measurement system 2000 according to an embodiment of the present disclosure. The system as shown includes a sample 2005, such as cell-free nucleic acid molecules (e.g., DNA and/or RNA) within an assay device 2010, where an assay 2008 can be performed on sample 2005. For example, sample 2005 can be contacted with reagents of assay 2008 to provide a signal (e.g., an intensity signal) of a physical characteristic 2015 (e.g., sequence information of a cell-free nucleic acid molecule). An example of an assay device can be a flow cell that includes probes and/or primers of an assay or a tube through which a droplet moves (with the droplet including the assay). Physical characteristic 2015 (e.g., a fluorescence intensity, a voltage, or a current), from the sample is detected by detector 2020. Detector 2020 can take a measurement at intervals (e.g., periodic intervals) to obtain data points that make up a data signal. In one embodiment, an analog-to-digital converter converts an analog signal from the detector into digital form at a plurality of times.

Assay device 2010 and detector 2020 can form an assay system, e.g., a PCR system or a sequencing system that performs sequencing according to embodiments described herein. A data signal 2025 is sent from detector 2020 to logic system 2030. As an example, data signal 2025 can be used to determine sequences and/or locations in a reference genome of nucleic acid molecules (e.g., DNA and/or RNA). Data signal 2025 can include various measurements made at a same time, e.g., different colors of fluorescent dyes or different electrical signals for different molecule of sample 2005, and thus data signal 2025 can correspond to multiple signals. Data signal 2025 may be stored in a local memory 2035, an external memory 2040, or a storage device 2045. The assay system can be comprised of multiple assay devices and detectors.

Logic system 2030 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2030 and the other components may be part of a stand-alone or network connected computer system, or they may be directly attached to or incorporated in a device (e.g., a sequencing device) that includes detector 2020 and/or assay device 2010. Logic system 2030 may also include software that executes in a processor 2050. Logic system 2030 may include a computer readable medium storing instructions for controlling measurement system 2000 to perform any of the methods described herein. For example, logic system 2030 can provide commands to a system that includes assay device 2010 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Logic system 2030 can perform any steps of methods described herein that perform computer processing.

Measurement system 2000 may also include a treatment device 2060, which can provide a treatment to the subject. Treatment device 2060 can determine a treatment and/or be used to perform a treatment. Examples of such treatment can include surgery, radiation therapy, chemotherapy, immunotherapy, targeted therapy, hormone therapy, and stem cell transplant. Logic system 2030 may be connected to treatment device 2060, e.g., to provide results of a method described herein. The treatment device may receive inputs from other devices, such as an imaging device and user inputs (e.g., to control the treatment, such as controls over a robotic system).

Measurement system 2000 may also include a reporting device 2055, which can present results of any of the methods describe herein, e.g., as determined using the measurement system. Reporting device 2055 can be in communication with a reporting module within logic system 2030 that can aggregate, format, and send a report to reporting device 2055. The reporting module can present information determined using any of the method described herein. The information can be presented by reporting device 2055 in any format that can be recognized and interpreted by a user of the measurement system 2000. For example, the information can be presented by reporting device 2055 in a displayed, printed, or transmitted format, or any combination thereof.

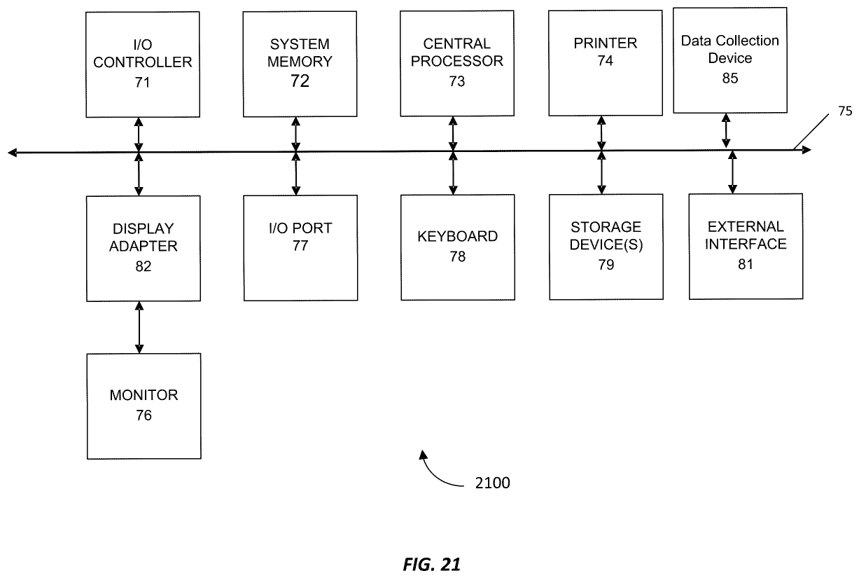

Any of the computer systems mentioned herein may utilize any suitable number of subsystems. Examples of such subsystems are shown in FIG. 21 in computer system 2100. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 21 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as prior art. Where a conflict exists between the instant application and a reference provided herein, the instant application shall dominate.

Informal Sequence Listing

SEQ ID NO: 1: Human DNASE1L3 original amino acid sequence
MSRELAPLLLLLLSIHSALAMRICSFNVRSFGESKQEDKNAMDVIVKVIKRCDIILVMEIK
DSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRW
KAENFIFMGDFNAGCSYVPKKAWKNIRLRTDPRFVWLIGDQEDTTVKKSTNCAYDRIV
LRGQEIVSSVVPKSNSVFDFQKAYKLTEEEALDVSDHFPVEFKLQSSRAFTNSKKSVTLR
KKTKSK
SEQ ID NO: 2: Human DNASEIL3 original nucleic acid sequence
ATGTCACGGGAGCTGGCCCCACTGCTGCTTCTCCTCCTCTCCATCCACAGCGCCCTG
GCCATGAGGATCTGCTCCTTCAACGTCAGGTCCTTTGGGGAAAGCAAGCAGGAAGA
CAAGAATGCCATGGATGTCATTGTGAAGGTCATCAAACGCTGTGACATCATACTCGT
GATGGAAATCAAGGACAGCAACAACAGGATCTGCCCCATACTGATGGAGAAGCTGA
ACAGAAATTCAAGGAGAGGCATAACGTACAACTATGTGATTAGCTCTCGGCTTGGA
AGAAACACATATAAAGAACAATATGCCTTTCTCTACAAGGAAAAGCTGGTGTCTGT
GAAGAGGAGTTATCACTACCATGACTATCAGGATGGAGACGCAGATGTGTTTTCCA
GGGAGCCCTTTGTGGTCTGGTTCCAATCTCCCCACACTGCTGTCAAAGACTTCGTGA
TTATCCCCCTGCACACCACCCCAGAGACATCCGTTAAGGAGATCGATGAGTTGGTTG
AGGTCTACACGGACGTGAAACACCGCTGGAAGGCGGAGAATTTCATTTTCATGGGT
GACTTCAATGCCGGCTGCAGCTACGTCCCCAAGAAGGCCTGGAAGAACATCCGCTT
GAGGACTGACCCCAGGTTTGTTTGGCTGATCGGGGACCAAGAGGACACCACGGTGA
AGAAGAGCACCAACTGTGCATATGACAGGATTGTGCTTAGAGGACAAGAAATCGTC
AGTTCTGTTGTTCCCAAGTCAAACAGTGTTTTTGACTTCCAGAAAGCTTACAAGCTG
ACTGAAGAGGAGGCCCTGGATGTCAGCGACCACTTTCCAGTTGAATTTAAACTACAG
TCTTCAAGGGCCTTCACCAACAGCAAAAAATCTGTCACTCTAAGGAAGAAAACAAA
GAGCAAACG
SEQ ID NO: 3: Codon optimized human DNASEIL3 nucleic acid sequence:
ATGTCTAGGGAACTTGCTCCACTCCTCTTGCTCCTCTTGTCTATCCACTCTGCTCTTGC
CATGCGTATCTGCTCTTTCAACGTCAGGTCTTTCGGTGAAAGCAAGCAGGAAGACAA
GAACGCTATGGACGTCATCGTGAAGGTCATCAAGAGGTGCGACATCATCCTCGTGAT
GGAAATCAAGGACTCAAACAACAGAATCTGTCCTATCCTGATGGAGAAGCTCAACC
GTAACTCCAGACGTGGAATCACCTACAACTACGTCATCTCTAGCAGATTGGGTCGTA
ACACTTACAAGGAACAATACGCCTTCTTGTACAAGGAGAAGCTGGTGTCAGTCAAG
AGGTCCTACCACTACCACGACTACCAGGACGGCGACGCTGACGTGTTCTCTCGCGAA
CCATTCGTCGTGTGGTTCCAGAGCCCTCACACCGCCGTGAAGGACTTCGTCATCATC
CCTTTGCACACCACTCCCGAAACCTCAGTGAAGGAGATCGACGAACTGGTGGAGGT
CTACACTGACGTCAAGCACAGATGGAAGGCTGAGAACTTCATCTTCATGGGTGACTT
CAACGCTGGCTGCTCTTACGTGCCCAAGAAGGCCTGGAAGAACATCAGGCTGCGCA
CCGACCCACGTTTCGTGTGGCTCATCGGCGACCAGGAAGACACCACTGTCAAGAAG
AGCACTAACTGTGCCTACGACAGGATCGTGCTGCGCGGACAGGAGATCGTCTCATC
CGTCGTGCCTAAGTCAAACTCCGTCTTCGACTTCCAAAAGGCTTACAAGCTCACTGA
AGAGGAAGCCTTGGACGTGAGCGACCACTTCCCCGTCGAATTCAAGCTCCAATCTA
GCAGAGCTTTCACAAACAGTAAAAAATCAGTAACCCTGCGTAAGAAGACTAAGAGT
AAACGTAGC

1. A method of increasing a cell-free DNA (cfDNA) level in a bodily fluid of a subject, the method comprising:
administering to the subject a therapeutically effective amount of a nuclease inhibitor, wherein the subject has been identified for a liquid biopsy to be assayed for cfDNA, and wherein the nuclease inhibitor binds to a nuclease and inhibits cleavage of the cfDNA in the bodily fluid, thereby increasing the cfDNA level in the subject.

2. The method of claim 1, wherein the nuclease inhibitor is an antibiotic.

3. The method of claim 2, wherein the antibiotic is a cephalosporin, sulfonamide, tetracycline, rifamycin, or polyene macrolide.

4. The method of claim 3, wherein the cephalosporin is ceforanide, cefuroxime sodium, cefamandole sodium, cefaclor, cefdinir, or a derivative thereof.

5. The method of claim 3, wherein the sulfonamide is sulfadiazine, sulphapyridine, or a derivative thereof.

6. The method of claim 3, wherein the tetracycline is minocycline, doxycycline, demeclocycline, tetracycline, omadacycline, sarecycline, or a derivative thereof.

7. The method of claim 3, wherein the rifamycin is rifaximin, rifabutin, rifamycin, rifampin (rifampicin), rifapentine, rifalazil, or a derivative thereof.

8. The method of claim 3, wherein the polyene macrolide is amphotericin B, nystatin, natamycin, or a derivative thereof.

9. The method of claim 1, wherein the nuclease is a deoxyribonuclease (DNase).

10. The method of claim 9, wherein the DNase is deoxyribonuclease 1 (DNase1) or deoxyribonuclease 1-like 1 (DNase1L1), DNase1L2, DNase1L3, or DNA fragmentation factor subunit beta (DFFB).

11. The method of claim 10, wherein the DNase is DNase1L3.

12. The method of claim 1, wherein the nuclease inhibitor binds to a catalytic pocket of the nuclease.

13. The method of claim 1, wherein the nuclease inhibitor is administered orally, intravenously, intradermally, intramuscularly, intraperitoneally, subcutaneously, intranasally, epidurally, sublingually, intracerebrally, intravaginally, trans-dermally, rectally, by inhalation, or topical administration.

14. The method of claim 1, wherein the therapeutically effective amount of the nuclease inhibitor is about 1-50 uM per kilogram body weight, optionally about 10-30 uM per kilogram body weight, and optionally about 20 uM per kilogram body weight.

15. The method of claim 1, wherein the cfDNA level is increased by up to 2, 3, 4, 5, 10, 25, 50, 100, 200, 300, 400, 500, 600, 700, 800, 900, or 1000-fold, as compared to a cfDNA level of a subject without administering the nuclease inhibitor.

16. The method of claim 1, wherein the bodily fluid is selected from the group consisting of blood, plasma, serum, urine, vaginal fluid, fluid from a hydrocele, vaginal flushing fluids, pleural fluid, ascitic fluid, cerebrospinal fluid, saliva, sweat, tears, sputum, bronchoalveolar lavage fluid, peritoneal fluid, discharge fluid from a nipple, aspiration fluid from different parts of the body, intraocular fluids, amniotic fluid, aqueous humor, ascites, bone marrow fluid, lymphatic fluid, synovial fluid, interstitial fluid, prostate fluid, semen, mucus, gastric acid, bile, pus, cerumen, breast milk, cowper's fluid or pre-ejaculatory fluid, female ejaculate, hair oil, cyst fluid, dialysis fluid, pericardial fluid, chyme, chyle, menses, sebum, vomit, mucosal secretion, stool water, pancreatic juice, lavage fluids from sinus cavities, bronchopulmonary aspirates, blastocoel fluid, urinary tract secretions, urethral secretions, bladder secretions, prostate secretions, vesical secretions, meconium, and umbilical cord fluid.

17. The method of claim 16, wherein the bodily fluid comprises blood, serum, and/or plasma.

18. The method of claim 1, wherein the subject is a human.

19. The method of claim 1, wherein the subject is pregnant, and wherein the cfDNA comprises a mixture of fetal and subject's DNA.

20. The method of claim 1, wherein the subject has, is suspected of having, or is at risk for a disease detectable using the cfDNA.

21. The method of claim 20, wherein the disease is cancer, a premalignant condition, an inflammatory disease, an immune disease, an autoimmune disease or disorder, a cardiovascular disease or disorder, neurological disease or disorder, infectious disease, or pain.

22. The method of claim 21, wherein the disease is cancer, and wherein the cancer is selected from the group consisting of colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, gallbladder cancer, brain cancer, peritoneal cancer, lymphoma, leukemia, multiple myeloma, neuroblastoma, osteosarcoma, head and neck cancer, oral cancer, nasopharyngeal cancer, skin cancer and soft tissue sarcoma.

23. A method of assaying cfDNA from a bodily fluid of a subject, comprising
(a) increasing cfDNA level in the bodily fluid of the subject by conducting the method of claim 1; and
(b) assaying the cfDNA.

24. The method of claim 23, further comprising collecting the cfDNA from the bodily fluid prior to the assaying step.

25. The method of claim 24, wherein the cfDNA is collected within 1 minute to 24 hours after the administering step.

26. The method of claim 23, wherein the assaying step comprises sequencing, amplification, hybridization, gel electrophoresis, chromatography, immunoassay, enzyme immunoassay (EIA), enzyme-linked immunosorbent assay (ELISA), enzyme-linked oligonucleotide assay (ELONA), affinity isolation, immunoprecipitation, Western blot, flow cytometry, and any combination thereof.

27. The method of claim 23, wherein the assaying step is conducted at least 1 minute, 2 minutes, 3 minutes, 4 minutes, 5 minutes, 10 minutes, 15 minutes, 30 minutes, 1 hour, 2 hours, 3 hours, 4 hours, 5 hours, 10 hours, 15 hours, 20 hours, or 24 hours after the administering step.

28. The method of claim 23, further comprising
(c) identifying the subject as having a disease based on a property of the cfDNA indicating the disease, wherein the property of the cfDNA is determined using assay results detected in the assaying step.

29. The method of claim 28, further comprising
(d) administering, to the subject, one or more treatments for the disease.

30. A method of inhibiting activity of a nuclease in a subject, the method comprising:
administering to the subject a therapeutically effective amount of a nuclease inhibitor that binds to the nuclease, thereby inhibiting the nuclease activity in the subject, wherein the subject has been identified for a liquid biopsy to be assayed for cfDNA.

31. The method of claim 30, wherein inhibiting the nuclease activity results in optimizing a level and/or a size of cfDNA to be assayed in the liquid biopsy.

32. The method of claim 30, wherein the nuclease inhibitor is an antibiotic.

33. The method of claim 32, wherein the antibiotic is selected from the group consisting of ceforanide, cefuroxime sodium, cefamandole sodium, cefaclor, cefdinir, sulfadiazine, sulphapyridine, minocycline, doxycycline, demeclocycline, tetracycline, omadacycline, sarecycline, rifaximin, rifabutin, rifamycin, rifampin (rifampicin), rifapentine, rifalazil, amphotericin B, nystatin, natamycin, and a derivative thereof.

34. The method of claim 33, wherein the antibiotic comprises ceforanide, cefuroxime sodium, minocycline, demeclocycline, and/or rifamycin.

35. The method of claim 30, wherein the nuclease inhibitor is administered orally, intravenously, intradermally, intramuscularly, intraperitoneally, subcutaneously, intranasally, epidurally, sublingually, intracerebrally, intravaginally, trans-dermally, rectally, by inhalation, or topical administration.

36. The method of claim 30, wherein the nuclease is a deoxyribonuclease (DNase).

37. The method of claim 36, wherein the DNase is deoxyribonuclease 1 (DNase1), deoxyribonuclease 1-like 1 (DNase1L1), DNase1L2, DNase1L3, or DNA fragmentation factor subunit beta (DFFB) nuclease.

38. The method of claim 30, wherein the nuclease activity is inhibited by about 5%, 10%, 15%, 20%, 25%, 30%, 35%, 40%, 45%, 50%, 55%, 60%, 65%, 70%, 75%, 80%, 85%, 90%, 95%, 99%, or 100%, as compared to the nuclease activity in the absence of the nuclease inhibitor.

39. A kit comprising a reagent for carrying out the method of claim 1.

40. The kit of claim 39, wherein the reagent comprises a nuclease inhibitor and a pharmaceutically acceptable adjuvant.

41. The kit of claim 40, wherein the pharmaceutically acceptable adjuvant comprises divalent cations Ca²⁺ and/or Mg²⁺.

42-75. (canceled)
